(19) United States
(12) Patent Application Publication
(10) Pub. No.: US 2009/0318479 A1
(43) Pub. Date: Dec. 24, 2009

(54) SUBSTITUTED RING FUSED AZINES AND THEIR USE IN CANCER THERAPY

(75) Inventors: William Alexander Denny, Auckland (NZ); Bruce Charles Baguley, Auckland (NZ); Elaine Shirley Marshall, Auckland (NZ); Hamish Scott Sutherland, Auckland (NZ)

Correspondence Address: OCCHIUTI ROHLICEK & TSAO, LLP, 10 FAWCETT STREET, CAMBRIDGE, MA, 02138, US

(73) Assignee: Auckland Uniservices Limited

(21) Appl. No.: 12/296,377

(22) PCT Filed: Apr. 4, 2007
(86) PCT No.: PCT/NZ2007/000077

§ 371 Date: Oct. 7, 2008

(30) Foreign Application Priority Data
Apr. 7, 2006 (NZ) 546477

(51) Int. Cl. A61K 31/517; C12Q 1/68; C07D 215/38; C07D 405/04; A61K 31/47

(52) U.S. Cl. 514/266.24; 435/6; 546/159; 544/293; 514/313

(57) ABSTRACT

The present invention relates to substituted ring fused azines and methods of using said compounds in treating cancers. More specifically, the present invention relates to the preparation of 4-alkyl-2-(heterocyclic)-azines and their use as cancer agents or drugs for cancer therapy. The compounds of the invention display favourable in vivo and in vitro activity against selected cancers.

### TECHNICAL FIELD

The present invention relates to 4-alkylamino-2-(heterocyclic)quinazolines, to their preparation, to their use as agents or drugs for cancer therapy, both alone or in combination with radiation and/or other anticancer drugs.

### BACKGROUND TO THE INVENTION

The tumour suppressor gene p53 codes for a DNA-binding transcription factor that plays a central role in gene regulation, and through this controls cell cycle progression and apoptosis. The corresponding p53 protein acts as a powerful tumor suppressor; knockout of the p53 gene in mice leads to the rapid formation of tumours [Chene, Exp. Opin. Ther. Pat., 2001, 11, 923]. The p53 gene is mutated in about half of all human cancers, largely by changes in the DNA binding domain that destabilize the loop-loop and loop-sheet-helix motif that form the DNA recognition surface [Cho et al., Science 1994, 346, 265]. This results in loss of tumour suppressor function [Hainaut & Hollstein et al., Adv. Cancer Res., 2000, 77, 81]. It was estimated in 1996 that such loss of p53 function accounts for about a third of all cancer incidence [Harris, J. Natl. Cancer Inst., 1996, 88, 1442].

One of the various approaches to combat the effects of this frequent loss of p53 function in human tumours is the use of small molecules that can stabilize the DNA binding domain of wild-type p53 in the active conformation, and in addition can bind to mutant forms of the protein and restore their active conformation and thus their function [Foster et al., Science, 1999, 286, 2507].

A large random screening programme identified a number of small hydrophobic compounds that were able to stabilise mutant p53 protein [Rastinejad et al., US 2002/0048271 A1, published Apr. 24, 20021. These included various linear tricyclic compounds and 2-styrylquinazolines. The structure-activity relationships were quite narrow, but the work identified in particular the 2-styrylquinazoline (i) reported in Foster et al., Science, 1999, 286, 2507.

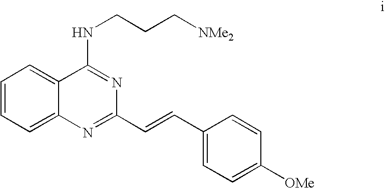

This compound restored the ability of mutant p53 protein to induce the cellular p21 gene in Saos-2 osteosarcoma cells, and was able to suppress the growth of A375.S2 melanoma (mutated at p53 position 249) and DLD-1 colon carcinoma (mutated at p53 position 241) cells in nude mice [Foster et al., Science, 1999, 286, 2507]. These compounds appear not to act on mature mis-folded protein, but on newly-synthesised p53. However, (i) is not very potent, and is also chemically unstable.

Related 2-([hetero]aryl)quinazolines have been generically claimed for the prevention of inflammatory diseases caused by bacterial DNA (Kisanuki et al., PCT. Intl. Appl. WO 02062767). These include the specifically claimed benzofuryl compound (ii).

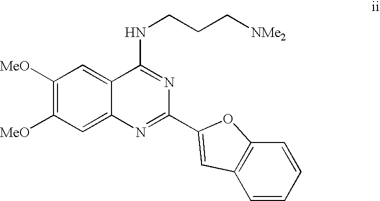

It is an object of the present invention to provide a class of 4-alkylamino-2-(heterocyclic)quinazolines as anticancer drugs, or to at least provide the public with a useful alternative.

### DISCLOSURE OF THE INVENTION

In a first aspect, the present invention provides a compound of Formula (I), wherein;

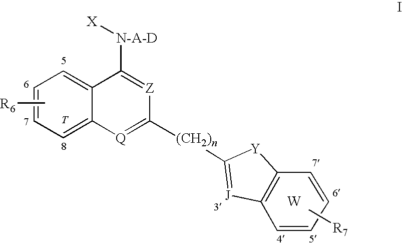

D is NR₁R₂ where R₁ and R₂ each independently represent H, lower C1-C6 alkyl or cycloalkyl optionally substituted with amino, hydroxyl or methoxy groups, or with one or more oxygen or nitrogen atoms as part of the cycloalkyl structure may represent morpholine, pyrrolidine, piperidine, imidazole or 4-methylpiperazine;
n may be 0, 1 or 2;
X may be H or lower C1-C6 alkyl or cycloalkyl optionally substituted with amino, hydroxyl or methoxy groups, or with one or more oxygen or nitrogen atoms as part of the cycloalkyl structure may represent may represent azetidine, pyrrolidine, piperidine, piperazine or morpholine;
Y may be O, CHR₃, S or, NR₄, where R₃ and R₄ may each independently represent H or lower C1-C6 alkyl or cycloalkyl optionally substituted with amino, hydroxyl or methoxy groups, or with one or more oxygen or nitrogen atoms as part of the cycloalkyl structure may represent azetidine, pyrrolidine, piperidine, piperazine or morpholine;
Z and Q may be N or CH, with the proviso that at least one of them is N;
J may be N or CR₅; where R₅ may represent H or lower C1-C6 alkyl or cycloalkyl optionally substituted with amino, hydroxyl or methoxy groups, or with one or more oxygen or nitrogen atoms as part of the cycloalkyl structure may represent azetidine, pyrrolidine, piperidine, piperazine or morpholine,
A is (CH₂)ₙ where n may be from 2 to 6, or A may together with D form a ring structure R₅ and R₇ at one or more of the available positions on rings T and W respectively, may at each occurrence independently represent one or more H, halogen, C1-C4 alkyl, C1-C4 alkenyl, C1-C4 alkynyl, OR₈, SR₈, NR₈R₉, CH₂R₈, COR₈, SOR₈, SO₂R₈, SO₂NR₈R₉, CO₂R₈, CONR₈R₉, CF₃, CN, or NO₂, where R₈ and R₉ may each independently represent H, lower C1-C6 alkyl or cycloalkyl optionally substituted with amino, hydroxyl or methoxy groups, or with one or more oxygen or nitrogen atoms as part of the cycloalkyl structure may represent azetidine, pyrrolidine, piperidine, piperazine or morpholine, or a physiologically acceptable salt or phosphate prodrug or carboxylic acid or aminoacid ester prodrug thereof.
with the proviso that the compound

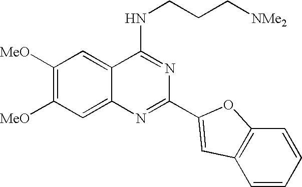

is excluded.

Preferably, when A together with D form a ring structure the ring structure is:

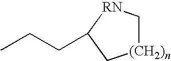

wherein n may be from 1 to 4 and R may represent a branched or unbranched C₁-C₆ alkyl.

Preferably, when A together with D form a ring structure the ring structure is

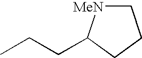

Preferably the compound of Formula I is a hydrochloride salt.

Preferably, the compound of formula I as defined above is selected from
N¹-[2-(1-benzofuran-2-yl)-4-quinazolinyl]-N²,N²-dimethyl-1,2-ethanediamine;
N¹-[2-(1-benzofuran-2-yl)-4-quinazolinyl]-N¹,N²,N²-trimethyl-1,2-ethanediamine
N¹-[2-(1-benzofuran-2-yl)-4-quinazolinyl]-N³,N³-dimethyl-1,3-propanediamine dihydrochloride;
N¹-[2-(1-benzofuran-2-yl)-4-quinazolinyl]-N⁴,N⁴-dimethyl-1,4-butanediamine dihydrochloride;
N¹-[2-(1-benzofuran-2-yl)-4-quinazolinyl]-N³,N³-diethyl-1,3-propanediamine dihydrochloride;
N¹-[2-(1-benzofuran-2-yl)-4-quinazolinyl]-N³,N³-dipropyl-1,3-propanediamine;
N¹-[2-(1-benzofuran-2-yl)-4-quinazolinyl]-N³,N³-bis(2-hydroxyethyl)-1,3-propanediamine;
2-(1-Benzofuran-2-yl)-N-[3-(4-morpholinyl)propyl]-4-quinazolinamine dihydrochloride;
2-(1-Benzofuran-2-yl)-N-[3-(4-methyl-1-piperazinyl)propyl]-4-quinazolinamine;
2-(1-benzofuran-2-yl)-N-[3-(1-pyrrolidinyl)propyl]-4-quinazolinamine dihydrochloride;
N¹-[2-(1-benzofuran-2-yl)-4-quinazolinyl]-N³-cyclopropyl-1,3-propanediamine dihydrochloride;
N¹-[2-(1-benzofuran-2-yl)-4-quinazolinyl]-N³-methyl-1,3-propanediamine dihydrochloride;
N¹-[2-(1-benzofuran-2-yl)-4-quinazolinyl]-N³-ethyl-1,3-propanediamine dihydrochloride;
N¹-[2-(1-Benzofuran-2-yl)-4-quinazolinyl]-N³,N³,2,2-tetramethyl-1,3-propanediamine dihydrochloride;
N¹-[2-(1-Benzofuran-2-yl)pyrido[3,2-d]pyrimidin-4-yl]-N³,N³-dimethyl-1,3-propanediamine dihydrochloride;
N¹-[2-(1-Benzofuran-2-yl)-5-methyl-4-quinazolinyl]-N³,N³-dimethyl-1,3-propanediamine dihydrochloride;
N¹-[2-(1-benzofuran-2-yl)-5-methoxy-4-quinazolinyl]-N³,N³-dimethyl-1,3-propanediamine dihydrochloride;
N¹-[2-(1-Benzofuran-2-yl)-5-chloro-4-quinazolinyl]-N³,N³-dimethyl-1,3-propanediamine dihydrochloride;
N¹-[2-(1-Benzofuran-2-yl)-5-nitro-4-quinazolinyl]-N³,N³-dimethyl-1,3-propanediamine;
N¹-[2-(1-Benzofuran-2-yl)-N⁴-[3-(dimethylamino)propyl]-4,5-quinazolinediamine dihydrochloride;
2-(1-benzofuran-2-yl)-N-[3-(dimethylamino)propyl]-4-{[3-(dimethylamino)propyl]amino}-5-quinazolinecarboxamide;
N¹-[2-(1-Benzofuran-2-yl)pyrido[4,3-d]pyrimidin-4-yl]-N³,N³-dimethyl-1,3-propanediamine;
N¹-[2-(1-Benzofuran-2-yl)-6-methyl-4-quinazolinyl]-N³,N³-dimethyl-1,3-propanediamine dihydrochloride;
N¹-[2-(1-Benzofuran-2-yl)-6-(trifluoromethyl)-4-quinazolinyl]-N³,N³-dimethyl-1,3-propanediamine;
N¹-[2-(1-Benzofuran-2-yl)-6-methoxy-4-quinazolinyl]-N³,N³-dimethyl-1,3-propanediamine dihydrochloride;
N¹-[2-(1-benzofuran-2-yl)-6-fluoro-4-quinazolinyl]-N³,N³-dimethyl-1,3-propanediamine;
N¹-[2-(1-Benzofuran-2-yl)-6-chloro-4-quinazolinyl]-N³,N³-dimethyl-1,3-propanediamine dihydrochloride;
N¹-[2-(1-benzofuran-2-yl)-6-bromo-4-quinazolinyl]-N³,N³-dimethyl-1,3-propanediamine dihydrochloride;
N¹-[2-(1-Benzofuran-2-yl)-6-nitro-4-quinazolinyl]-N³,N³-dimethyl-1,3-propanediamine;
N¹-[2-(1-Benzofuran-2-yl)-N⁴-[3-(dimethylamino)propyl]-4,6-quinazolinediamine dihydrochloride;
2-(1-benzofuran-2-yl)-4-{[3-(dimethylamino)propyl]amino}-6-quinazolinecarbonitrile;
2-(1-benzofuran-2-yl)-4-{[3-(dimethylamino)propyl]amino}-6-quinazolinecarboxamide dihydrochloride;
N¹-[2-(1-Benzofuran-2-yl)pyrido[3,4-d]pyrimidin-4-yl]-N³,N³-dimethyl-1,3-propanediamine dihydrochloride;
N¹-[2-(1-benzofuran-2-yl)-7-methyl-4-quinazolinyl]-N³,N³-dimethyl-1,3-propanediamine dihydrochloride;
N¹-[2-(1-Benzofuran-2-yl)-7-(trifluoromethyl)-4-quinazolinyl]-N³,N³-dimethyl-1,3-propanediamine dihydrochloride;
N¹-[2-(1-Benzofuran-2-yl)-7-methoxy-4-quinazolinyl]-N³,N³-dimethyl-1,3-propanediamine dihydrochloride;
N¹-[2-(1-benzofuran-2-yl)-7-fluoro-4-quinazolinyl]-N³,N³-dimethyl-1,3-propanediamine dihydrochloride;
N¹-[2-(1-Benzofuran-2-yl)-7-chloro-4-quinazolinyl]-N³,N³-dimethyl-1,3-propanediamine dihydrochloride;
N¹-[2-(1-benzofuran-2-yl)-7-bromo-4-quinazolinyl]-N³,N³-dimethyl-1,3-propanediamine dihydrochloride;
N¹-[2-(1-Benzofuran-2-yl)-7-nitro-4-quinazolinyl]-N³,N³-dimethyl-1,3-propanediamine;
N¹-[2-(1-Benzofuran-2-yl)-7-amino-4-quinazolinyl]-N³,N³-dimethyl-1,3-propanediamine dihydrochloride;
2-(1-benzofuran-2-yl)-4-[3-(dimethylamino)propyl]amino)-7-quinazolinecarbonitrile;
2-(1-benzofuran-2-yl)-4-{[3-(dimethylamino)propyl]amino}7-quinazolinecarboxamide dihydrochloride;
N¹-[2-(1-Benzofuran-2-yl)pyrido[2,3-a]pyrimidin-4-yl]-N³,N³-dimethyl-1,3-propanediamine dihydrochloride;
N¹-[2-(1-Benzofuran-2-yl)-8-methyl-4-quinazolinyl]-N³,N³-dimethyl-1,3-propanediamine dihydrochloride;
N¹-[2-(1-Benzofuran-2-yl)-8-phenyl-4-quinazolinyl]-N³,N³-dimethyl-1,3-propanediamine dihydrochloride;
N¹-[2-(d-Benzofuran-2-yl)-8-(trifluoromethyl)-4-quinazolinyl]-N³,N³-dimethyl-1,3-propanediamine dihydrochloride;
N¹-[2-(1-Benzofuran-2-yl)-8-methoxy-4-quinazolinyl]-N³,N³-dimethyl-1,3-propanediamine dihydrochloride;
N¹-[2-(1-Benzofuran-2-yl)-8-chloro-4-quinazolinyl]-N³,N³-dimethyl-1,3-propanediamine dihydrochloride;
N¹-[2-(1-Benzofuran-2-yl)-8-nitro-4-quinazolinyl]-N³,N³-dimethyl-1,3-propanediamine;
N¹-[2-(1-Benzofuran-2-yl)-8-amino-4-quinazolinyl]-N³,N³-dimethyl-1,3-propanediamine dihydrochloride;
2-(1-benzofuran-2-yl)-4-{[3-(dimethylamino)propyl]amino}-8-quinazolinecarbonitrile;
2-(1-benzofuran-2-yl)-4-{[3-(dimethylamino)propyl]amino}8-quinazolinecarboxamide;
N¹-[2-(1-Benzofuran-2-yl)benzo[g]quinazolin-4-yl]-N³,N³-dimethyl-1,3-propanediamine dihydrochloride;
N¹-[2-(1-Benzofuran-2-yl)-6,7-dichloro-4-quinazolinyl]-N³,N³-dimethyl-1,3-propanediamine dihydrochloride;
N¹-[2-(1-Benzofuran-2-yl)-6,8-dichloro-4-quinazolinyl]-N³,N³-dimethyl-1,3-propanediamine dihydrochloride;
N¹-[2-(1-Benzofuran-2-yl)-6,8-dibromo-4-quinazolinyl]-N³,N³-dimethyl-1,3-propanediamine;
N¹-[2-(1-Benzofuran-2-yl)-7,8-dimethyl-4-quinazolinyl]-N³,N³-dimethyl-1,3-propanediamine dihydrochloride;
N¹-[2-(1-Benzofuran-2-yl)-7,8-dimethoxy-4-quinazolinyl]-N³,N³-dimethyl-1,3-propanediamine dihydrochloride;
N¹,N¹-Dimethyl-N³-[2-(3-methyl-1-benzofuran-2-yl)-4-quinazolinyl]-1,3-propanediamine dihydrochloride;
N¹-[2-(4 chloro-5-methoxy-1-benzofuran-2-yl)-4-quinazolinyl]-N¹,N¹-dimethyl-1,3-propanediamine hydrochloride;
N¹-[2-(5-methoxy-1-benzofuran-2-yl)-4-quinazolinyl]-N¹,N¹-dimethyl-1,3-propanediamine;
N¹,N¹-dimethyl-N³-[2-(5-methyl-1-benzofuran-2-yl)-4-quinazolinyl]-1,3-propanediamine dihydrochloride;
N¹,N¹-dimethyl-N³-[2-(5-chloro-1-benzofuran-2-yl)-4-quinazolinyl]-1,3-propanediamine;
N¹-[2-(5-Bromo-1-benzofuran-2-yl)-4-quinazolinyl]-N³,N³-dimethyl-1,3-propanediamine;
N¹-[2-(6-Methoxy-1-benzofuran-2-yl)-4-quinazolinyl]-N¹,N¹-dimethyl-1,3-propanediamine dihydrochloride;
N¹,N¹-dimethyl-N³-[2-(7-methyl-1-benzofuran-2-yl)-4-quinazolinyl]-1,3-propanediamine;
N¹,N¹-dimethyl-N³-[2-(7-methoxy-1-benzofuran-2-yl)-4-quinazolinyl]-1,3-propanediamine dihydrochloride;
N¹,N¹-Dimethyl-N³-[8-methyl-2-(3-methyl-1-benzofuran-2-yl)-4-quinazolinyl]-1,3-propanediamine dihydrochloride;
N¹-[2-(6-Methoxy-1H-indol-2-yl)-4-quinazolinyl]-N³,N³-dimethyl-1,3-propanediamine dihydrochloride;
N¹,N¹-Dimethyl-N³-[2-(5-methoxy-1-methyl-1H-indol-2-yl)-4-quinazolinyl]-1,3-propanediamine dihydrochloride;
N¹-[2-(6-Methoxy-1-benzofuran-2-yl)-4-quinazolinyl]-N¹,N¹-dimethyl-1,3-propanediamine dihydrochloride;
N¹-[2-(1H-Indol-2-yl)-4-quinazolinyl]-N³,N³-dimethyl-1,3-propanediamine dihydrochloride;
N¹-[2-(1H-Indol-2-yl)-4-quinazolinyl]-N-[3-(4-morpholinyl)propyl]amine dihydrochloride;
N¹,N¹-Dimethyl-N³-[2-(1-methyl-1H-indol-2-yl)-4-quinazolinyl]-1,3-propanediamine dihydrochloride;
N¹-[2-(1-Benzothien-2-yl)-4-quinazolinyl]-N³,N³-dimethyl-1,3-propanediamine dihydrochloride;
N¹,N¹-Dimethyl-N³-[2-(3-quinolinyl)-4-quinazolinyl]-1,3-propanediamine dihydrochloride;
N¹,N¹-Dimethyl-N³-[2-(2-naphthyl)-4-quinazolinyl]-1,3-propanediamine dihydrochloride;
2-(1-Benzofuran-2-yl)-N³-[2-(1-methyl-2-pyrrolidinyl)ethyl]-4-quinazolinamine dihydrochloride;
2-(1-Benzofuran-2-yl)-7,8-dimethyl-N-[2-(1-methyl-2-pyrrolidinyl)ethyl]-4-quinazolinamine dihydrochloride;
N¹-[2-(1-benzofuran-2-yl)-4-quinolinyl]-N³,N³-dimethyl-1,3-propanediamine dihydrochloride and
N¹-[3-(1-benzofuran-2-yl)-1-isoquinolinyl]-N³,N³-dimethyl-1,3-propanediamine dihydrochloride.

It is appreciated that compounds of Formula I may occur in different geometric and enantiomeric forms, and that both pure forms and mixtures of these separate isomers are included, and any physiologically functional salt derivatives or phosphate or carboxylic acid or aminoacid ester prodrugs thereof.

A preferred subclass of the invention is where, in Formula I:

D is NR₁R₂ where R₁ and R₂ each independently represent H, lower C1-C6 alkyl or cycloalkyl, where one or more oxygen or nitrogen atoms as part of the cycloalkyl structure may represent azetidine, pyrrolidine, piperidine, piperazine or morpholine
n may be 0 or 1;
X may be H or lower C1-C6 alkyl or cycloalkyl;

### Y may be O or S;

### Both Z and Q are N;

### J may be CH or C-Me;

A is (CH₂)ₙ where n may be from 2 to 4, or A may together with D form a ring structure;
R₆ and R₇ at the 6-, 7- or 8-positions on ring T and at the 3′-position on ring W respectively, may at each occurrence independently represent one or more H, halogen, C1-C4 alkyl, C1-C4 alkenyl, C1-C4 alkynyl, SR₈, NR₈R₉, CH₂R₈, COR₈, SOR₈, SO₂R₈, SO₂NR₈R₉, CO₂R₈, CONR₈R₉, CF₃, CN, or NO₂, where R₈ and R₉ may each independently represent H or lower C1-C6 alkyl or cycloalkyl optionally substituted with amino, hydroxyl or methoxy groups;
or a physiologically acceptable salt or phosphate prodrug or carboxylic acid or aminoacid ester prodrug thereof.

A specially preferred subclass of the invention is where, in Formula I;

D is NR₁R₂ where R₁ and R₂ each independently represent H or lower C1-C6 alkyl or cycloalkyl;
n is 0;

### X is H;

### Y is O;

### Both Z and Q are N;

### J is CH;

### A is (CH₂)₃;

R₆ and R₇ at the 6-, 7- or 8-positions on ring T and at the 3′ positions on ring W respectively, may at each occurrence independently represent one or more H, halogen, C₁-C₄ alkyl, CF₃, NO₂ and NH₂;
or a physiologically acceptable salt or phosphate prodrug or carboxylic acid or aminoacid ester prodrug thereof.

In a second aspect the invention provides a method of cancer prevention or therapy for treating cancers including the step of administering a compound of Formula I wherein;

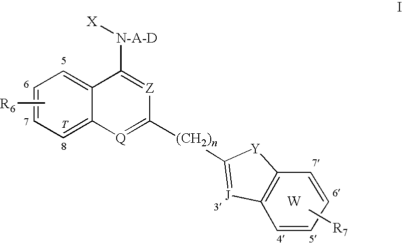

D is NR₁R₂ where R₁ and R₂ each independently represent H, lower C1-C6 alkyl or cycloalkyl optionally substituted with amino, hydroxyl or methoxy groups, or with one or more oxygen or nitrogen atoms as part of the cycloalkyl structure may represent morpholine, pyrrolidine, piperidine, imidazole or 4-methylpiperazine;
n may be 0, 1 or 2;
X may be H or lower C1-C6 alkyl or cycloalkyl optionally substituted with amino, hydroxyl or methoxy groups, or with one or more oxygen or nitrogen atoms as part of the cycloalkyl structure may represent may represent azetidine, pyrrolidine, piperidine, piperazine or morpholine;
Y may be O, CHR₃, S or, NR₄, where R₃ and R₄ may each independently represent H or lower C1-C6 alkyl or cycloalkyl optionally substituted with amino, hydroxyl or methoxy groups, or with one or more oxygen or nitrogen atoms as part of the cycloalkyl structure may represent azetidine, pyrrolidine, piperidine, piperazine or morpholine;
Z and Q may be N or CH, with the proviso that at least one of them is N;
J may be N or CR₅; where R₅ may represent H or lower C1-C6 alkyl or cycloalkyl optionally substituted with amino, hydroxyl or methoxy groups, or with one or more oxygen or nitrogen atoms as part of the cycloalkyl structure may represent azetidine, pyrrolidine, piperidine, piperazine or morpholine,
A is (CH₂)ₙ where n may be from 2 to 6, or A may together with D form a ring structure R₆ and R₇ at one or more of the available positions on rings T and W respectively, may at each occurrence independently represent one or more H, halogen, C1-C4 alkyl, C1-C4 alkenyl, C1-C4 alkynyl, OR₈, SR₈, NR₈R₉, CH₂R₈, COR₈, SOR₈, SO₂R₈, SO₂NR₈R₉, CO₂R₈, CONR₈R₉, CF₃, CN, or NO₂, where R₈ and R₉ may each independently represent H, lower C1-C6 alkyl or cycloalkyl optionally substituted with amino, hydroxyl or methoxy groups, or with one or more oxygen or nitrogen atoms as part of the cycloalkyl structure may represent azetidine, pyrrolidine, piperidine, piperazine or morpholine,
or a physiologically acceptable salt or phosphate prodrug or carboxylic acid or aminoacid ester prodrug thereof.

Preferably, the subject is in need of restoration of its cell arrest function. More preferably at least 10% of the expected level of normal range of cell arrest function is restored in the subject. Most preferably at least 50% of the expected level of normal range of cell arrest function is restored in the subject.

It is to be understood that reference to the terms “restoration”, “restored” or “restoring” of cell arrest function throughout the specification is intended to include situations where at least 10% of the expected level of normal range of cell arrest function is restored. The expected normal range of cell arrest function would be the level of function that one would see in a given subject in the absence of any loss of cell arrest function. It is envisaged that with as little as 10% restoration of cell arrest function that the feedback loop(s) involved in the cell arrest pathway(s) will be activated and will enable the general establishment of the cell arrest functions.

Preferably the method further includes also administering one or more chemotherapeutic agents and/or therapies selected from:

Cisplatin or other platinum-based derivatives,
Temozolomide or other DNA methylating agents,
Cyclophosphamide or other DNA alkylating agents,
Doxorubicin, mitoxantrone, camptothecin or other topoisomerase inhibitors,
Methotrexate, gemcitabine or other antimetabolites;
Docetaxel or other taxanes; kinase inhibitors and radiotherapy.

It is preferred that the method of therapy further includes the step of administering one or more chemotherapeutic agents to the subject before, during or after the administration of the compound of Formula I as defined above in the second aspect of the invention to the subject.

While these compounds will typically be used in cancer prevention or cancer therapy of human subjects, they can be used to target cancer cells in other warm blooded animal subjects such as other primates, farm animals such as cattle, and sports animals and pets such as horses, dogs, and cats.

In a third aspect of the present invention there is provided a pharmaceutical composition including a therapeutically effective amount of a compound of formula I as defined above in the second aspect of the invention, and a pharmaceutically acceptable excipient, adjuvant, carrier, buffer or stabiliser.

A “therapeutically effective amount”, is to be understood as an amount of a compound of Formula I as defined above in the first or second aspects of the invention that is sufficient to show some restoration of the function of the cell arrest functions. The actual amount, rate and time-course of administration, will depend on the nature and severity of the disease being treated. Prescription of treatment is within the responsibility of general practitioners and other medical doctors.

The pharmaceutically acceptable excipient, adjuvant, carrier, buffer or stabiliser should be non-toxic and should not interfere with the efficacy of the active ingredient. The precise nature of the carrier or other material will depend on the route of administration, which may be oral, or by injection, such as cutaneous, subcutaneous, or intravenous injection.

Pharmaceutical compositions for oral administration may be in tablet, capsule, powder or liquid form. A tablet may comprise a solid carrier or an adjuvent. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such as gelatin.

For intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as Sodium Chloride injection, Ringer's injection, Lactated Ringer's injection. Preservatives, stabilisers, buffers antioxidants and/or other additives may be included as required.

In a fourth aspect, there is provided the use in the manufacture of a medicament of a therapeutically effective amount of a compound of Formula as defined above in the first or second aspects of the invention for administration to a subject.

Preferably the subject is in need of restoration of its cell arrest function.

In a fifth aspect of the present invention there is provided a method of making a compound of formula I the method including the steps of reacting a 2-aryl-4-chloroquinazoline of formula II with an amine

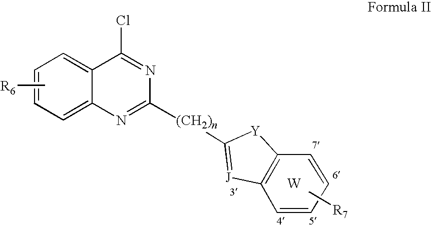

wherein variables R₆, R₇, J, n and Y are as defined above for Formula I.

In a further embodiment, the method includes the steps of making a compound of Formula II, including the step of chlorination of a compound of Formula III

wherein variables R₆, R₇, J, n and Y are as defined above for Formula I

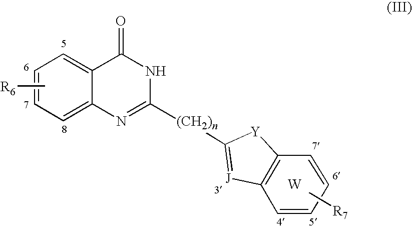

In a further embodiment, the method includes the steps of making a compound of Formula III as defined above, the method including one of the following steps;
(i) by boronic acid (Suzuki) coupling as illustrated in Scheme 1 below
(ii) by amination of a substituted anthranilate ester, followed by a cyclisation step as illustrated in Scheme 2 below;
(iii) by cyclisation of a substituted anthranilamide as illustrated in Scheme 3 below or

In a further aspect there is provided a compound of Formula I obtained by the methods defined above.

Preferably the compound of Formula I obtained by the method defined above is selected from one or more of the following:
N¹-[2-(1-Benzofuran-2-yl)-4-quinazolinyl]-N²,N²-dimethyl-1,2-ethanediamine;
N¹-[2-(1-Benzofuran-2-yl)-4-quinazolinyl]-N²,N²-trimethyl-1,2-ethanediamine
N¹-[2-(1-Benzofuran-2-yl)-4-quinazolinyl]-N³,N³-dimethyl-1,3-propanediamine dihydrochloride;
N¹-[2-(1-Benzofuran-2-yl)-4-quinazolinyl]-N4,N-dimethyl-1,4-butanediamine dihydrochloride;
N¹-[2-(1-Benzofuran-2-yl)-4-quinazolinyl]-N³,N³-diethyl-1,3-propanediamine dihydrochloride;
N¹-[2-(1-Benzofuran-2-yl)-4-quinazolinyl]-N³,N³-dipropyl-1,3-propanediamine;
N¹-[2-(1-Benzofuran-2-yl)-4-quinazolinyl]-N³,N³-bis(2-hydroxyethyl)-1,3-propanediamine;
2-(1-Benzofuran-2-yl)-N-[3-(4-morpholinyl)propyl]-4-quinazolinamine dihydrochloride;
2-(1-Benzofuran-2-yl)-N-[3-(4-methyl-1-piperazinyl)propyl]-4-quinazolinamine;
2-(1-Benzofuran-2-yl)-N-[3-(1-pyrrolidinyl)propyl]-4-quinazolinamine dihydrochloride;
N¹-[2-(1-Benzofuran-2-yl)-4-quinazolinyl]-N³-cyclopropyl-1,3-propanediamine dihydrochloride;
N¹-[2-(1-Benzofuran-2-yl)-4-quinazolinyl]-N³-methyl-1,3-propanediamine dihydrochloride;
N¹-[2-(1-Benzofuran-2-yl)-4-quinazolinyl]-N³-ethyl-1,3-propanediamine dihydrochloride;
N¹-[2-(1-Benzofuran-2-yl)-4-quinazolinyl]-N³,N³,2,2-tetramethyl-1,3-propanediamine dihydrochloride;
N¹-[2-(1-Benzofuran-2-yl)pyrido[3,2-o]pyrimidin-4-yl]-N³,N³-dimethyl-1,3-propanediamine dihydrochloride;
N¹-[2-(1-Benzofuran-2-yl)-5-methyl-4-quinazolinyl]-N³,N³-dimethyl-1,3-propanediamine dihydrochloride;
N¹-[2-(1-Benzofuran-2-yl)-5-methoxy-4-quinazolinyl]-N³,N³-dimethyl-1,3-propanediamine dihydrochloride;
N¹-[2-(1-Benzofuran-2-yl)-5-chloro-4-quinazolinyl]-N³,N³-dimethyl-1,3-propanediamine dihydrochloride;
N¹-[2-(1-Benzofuran-2-yl)-5-nitro-4-quinazolinyl]-N³,N³-dimethyl-1,3-propanediamine;
N¹-[2-(1-Benzofuran-2-yl)-N⁴-[3-(dimethylamino)propyl]-4,5-quinazolinediamine dihydrochloride;
2-(1-Benzofuran-2-yl)-N-[3-(dimethylamino)propyl]-4-{[3-(dimethylamino)propyl]amino}-5-quinazolinecarboxamide;
N¹-[2-(1-Benzofuran-2-yl)pyrido[4,3-o]pyrimidin-4-yl]-N³,N³-dimethyl-1,3-propanediamine;
N¹-[2-(1-Benzofuran-2-yl)-6-methyl-4-quinazolinyl]-N³,N³-dimethyl-1,3-propanediamine dihydrochloride;
N¹-[2-(1-Benzofuran-2-yl)-6-(trifluoromethyl)-4-quinazolinyl]-N³,N³-dimethyl-1,3-propanediamine;
N¹-[2-(1-Benzofuran-2-yl)-6-methoxy-4-quinazolinyl]-N³,N³-dimethyl-1,3-propanediamine dihydrochloride;
N¹-[2-(1-Benzofuran-2-yl)-6-fluoro-4-quinazolinyl]-N³,N³-dimethyl-1,3-propanediamine;
N¹-[2-(1-Benzofuran-2-yl)-6-chloro-4-quinazolinyl]-N³,N³-dimethyl-1,3-propanediamine dihydrochloride;
N¹-[2-(1-Benzofuran-2-yl)-6-bromo-4-quinazolinyl]-N³,N³-dimethyl-1,3-propanediamine dihydrochloride;
N¹-[2-(1-Benzofuran-2-yl)-6-nitro-4-quinazolinyl]-N³,N³-dimethyl-1,3-propanediamine;
N¹-[2-(1-Benzofuran-2-yl)-N⁴-[3-(dimethylamino)propyl]-4,6-quinazolinediamine dihydrochloride;
2-(1-Benzofuran-2-yl)-4-{[3-(dimethylamino)propyl]amino}-6-quinazolinecarbonitrile;
2-(1-Benzofuran-2-yl)-4-{[3-(dimethylamino)propyl]amino}-6-quinazolinecarboxamide dihydrochloride;
N¹-[2-(1-Benzofuran-2-yl)pyrido[3,4-a]pyrimidin-4-yl]-N³,N³-dimethyl-1,3-propanediamine dihydrochloride;
N¹-[2-(1-Benzofuran-2-yl)-7-methyl-4-quinazolinyl]-N³,N³-dimethyl-1,3-propanediamine dihydrochloride;
N¹-[2-(1-Benzofuran-2-yl)-7-(trifluoromethyl)-4-quinazolinyl]-N³,N³-dimethyl-1,3-propanediamine dihydrochloride;
N¹-[2-(1-Benzofuran-2-yl)-7-methoxy-4-quinazolinyl]-N³,N³-dimethyl-1,3-propanediamine dihydrochloride;
N¹-[2-(1-Benzofuran-2-yl)-7-fluoro-4-quinazolinyl]-N³,N³-dimethyl-1,3-propanediamine dihydrochloride;
N¹-[2-(1-Benzofuran-2-yl)-7-chloro-4-quinazolinyl]-N³,N³-dimethyl-1,3-propanediamine dihydrochloride;
N¹-[2-(1-Benzofuran-2-yl)-7-bromo-4-quinazolinyl]-N³,N³-dimethyl-1,3-propanediamine dihydrochloride;
N¹-[2-(1-Benzofuran-2-yl)-7-nitro-4-quinazolinyl]-N³,N³-dimethyl-1,3-propanediamine;
N¹-[2-(1-Benzofuran-2-yl)-7-amino-4-quinazolinyl]-N³,N³-dimethyl-1,3-propanediamine dihydrochloride;
2-(1-Benzofuran-2-yl)-4-([3-(dimethylamino)propyl]amino)-7-quinazolinecarbonitrile;
2-(1-Benzofuran-2-yl)-4-{[3-(dimethylamino)propyl]amino}-7-quinazolinecarboxamide dihydrochloride;
N¹-[2-(1-Benzofuran-2-yl)pyrido[2,3d]pyrimidin-4-yl]-N³,N³-dimethyl-1,3-propanediamine dihydrochloride;
N¹-[2-(1-Benzofuran-2-yl)-8-methyl-4-quinazolinyl]-N³,N³-dimethyl-1,3-propanediamine dihydrochloride;
N¹-[2-(1-Benzofuran-2-yl)-8-phenyl-4-quinazolinyl]-N³,N³-dimethyl-1,3-propanediamine dihydrochloride;
N¹-[2-(1-Benzofuran-2-yl)-8-(trifluoromethyl)-4-quinazolinyl]-N³,N³-dimethyl-1,3-propanediamine dihydrochloride;
N¹-[2-(1-Benzofuran-2-yl)-8-methoxy-4-quinazolinyl]-N³,N³-dimethyl-1,3-propanediamine dihydrochloride;
N¹-[2-(1-Benzofuran-2-yl)-8-chloro-4-quinazolinyl]-N³,N³-dimethyl-1,3-propanediamine dihydrochloride;
N¹-[2-(1-Benzofuran-2-yl)-8-nitro-4-quinazolinyl]-N³,N³-dimethyl-1,3-propanediamine;
2-(1-Benzofuran-2-yl)-4-{[3-(dimethylamino)propyl]amino}-8-quinazolinecarbonitrile;
2-(1-Benzofuran-2-yl)-4-{[3-(dimethylamino)propyl]amino}-8-quinazolinecarboxamide;
N¹-[2-(1-Benzofuran-2-yl)benzo[g]quinazolin-4-yl]-N³,N³-dimethyl-1,3-propanediamine dihydrochloride;
N¹-[2-(1-Benzofuran-2-yl)-6,7-dichloro-4-quinazolinyl]-N³,N³-dimethyl-1,3-propanediamine dihydrochloride;
N¹-[2-(1-Benzofuran-2-yl)-6,8-dichloro-4-quinazolinyl]-N³,N³-dimethyl-1,3-propanediamine dihydrochloride;
N¹-[2-(1-Benzofuran-2-yl)-6,8-dibromo-4-quinazolinyl]-N³,N³-dimethyl-1,3-propanediamine;
N¹-[2-(1-Benzofuran-2-yl)-7,8-dimethyl-4-quinazolinyl]-N³,N³-dimethyl-1,3-propanediamine dihydrochloride;
N¹-[2-(1-Benzofuran-2-yl)-7,8-dimethoxy-4-quinazolinyl]-N³,N³-dimethyl-1,3-propanediamine dihydrochloride;
N¹,N¹-Dimethyl-N³-[2-(3-methyl-1-benzofuran-2-yl)-4-quinazolinyl]-1,3-propanediamine dihydrochloride;
N¹-[2-(4-Chloro-5-methoxy-1-benzofuran-2-yl)-4-quinazolinyl]-N¹,N¹-dimethyl-1,3-propanediamine hydrochloride;
N¹-[2-(5-Methoxy-1-benzofuran-2-yl)-4-quinazolinyl]-N¹,N¹-dimethyl-1,3-propanediamine;
N¹,N¹-Dimethyl-N³-[2-(5-methyl-1-benzofuran-2-yl)-4-quinazolinyl]-1,3-propanediamine dihydrochloride;
N¹,N¹-Dimethyl-N³-[2-(5-chloro-1-benzofuran-2-yl)-4-quinazolinyl]-1,3-propanediamine;
N¹-[2-(5-Bromo-1-benzofuran-2-yl)-4-quinazolinyl]-N³,N³-dimethyl-1,3-propanediamine;
N¹-[2-(6-Methoxy-1-benzofuran-2-yl)-4-quinazolinyl]-N¹,N¹-dimethyl-1,3-propanediamine dihydrochloride;
N¹,N¹-Dimethyl-N³-[2-(7-methyl-1-benzofuran-2-yl)-4-quinazolinyl]-1,3-propanediamine;
N¹,N¹-Dimethyl-N³-[2-(7-methoxy-1-benzofuran-2-yl)-4-quinazolinyl]-1,3-propanediamine dihydrochloride;
N¹,N¹-Dimethyl-N³-[8-methyl-2-(3-methyl-1-benzofuran-2-yl)-4-quinazolinyl]-1,3-propanediamine dihydrochloride;
N¹-[2-(5-Methoxy-1H-indol-2-yl)-4-quinazolinyl]-N³N³-dimethyl-1,3-propanediamine dihydrochloride;
N¹,N¹-Dimethyl-N³-[2-(5-methoxy-1-methyl-1H-indol-2-yl)-4-quinazolinyl]-1,3-propanediamine dihydrochloride;
N¹-[2-(6-Methoxy-1-benzofuran-2-yl)-4-quinazolinyl]-N¹,N¹-dimethyl-1,3-propanediamine dihydrochloride;
N¹-[2-(1H-Indol-2-yl)-4-quinazolinyl]-N³,N³-dimethyl-1,3-propanediamine dihydrochloride;
N¹-[2-(1H-Indol-2-yl)-4-quinazolinyl]-N-[3-(4-morpholinyl)propyl]amine dihydrochloride;
N¹,N¹-Dimethyl-N³-[2-(1-methyl-1H-indol-2-yl)-4-quinazolinyl]-1,3-propanediamine dihydrochloride;
N¹-[2-(1-Benzothien-2-yl)-4-quinazolinyl]-N³,N³-dimethyl-1,3-propanediamine dihydrochloride;
N¹,N¹-Dimethyl-N³-[2-(3-quinolinyl)-4-quinazolinyl]-1,3-propanediamine dihydrochloride;
N¹,N¹-Dimethyl-N³-[2-(2-naphthyl)-4-quinazolinyl]-1,3-propanediamine dihydrochloride;
2-(1-Benzofuran-2-yl)-N³-[2-(1-methyl-2-pyrrolidinyl)ethyl]-4-quinazolinamine dihydrochloride;
2-(1-Benzofuran-2-yl)-7,8-dimethyl-N-[2-(1-methyl-2-pyrrolidinyl)ethyl]-4-quinazolinamine dihydrochloride;
N¹-[2-(1-Benzofuran-2-yl)-4-quinolinyl]-N³,N³-dimethyl-1,3-propanediamine dihydrochloride and
N¹-[3-(1-Benzofuran-2-yl)-1-isoquinolinyl]-N³,N³-dimethyl-1,3-propanediamine dihydrochloride.

In a further aspect, the present invention provides an assay for determining the restoration of cell arrest function including the steps of
(a) plating and culturing one or more tumour cell lines in growth media under cell culture conditions,
(b) adding a compound of Formula I as defined above to one or more of the cultures,
(c) adding an inhibitor of cell division to one or more of the cultures,
(d) irradiating one or more of the cultures,
(e) incubating, harvesting, and
(f) analyzing the cellular DNA content profiles to estimate the proportions of G1-S- and G₂/M-phase cells in the cultures.

It is to be recognised that certain compounds of the present invention may exist in one or more different enantiomeric or diastereomeric forms. It is to be understood that the enantiomeric or diasteriomeric forms are included in the above aspects of the invention.

The term halo or halogen group used throughout the specification is to be taken as meaning a fluoro, chloro, bromo or iodo group.

It is to be understood that where variables of the Formula I or II as defined above are optionally substituted by one or more imidazolyl, piperazinyl, morpholinyl, piperidinyl, azepanyl, pyrrolidinyl or azetidinyl groups that the linkage to the relevant variable may be through either one of the available nitrogen or carbon ring atoms of these groups.

The term pharmacologically acceptable salt used throughout the specification is to be taken as meaning any acid or base derived salt formed from hydrochloric, sulfuric, phosphoric, acetic, citric, oxalic, malonic, salicylic, malic, fumaric, succinic, ascorbic, maleic, methanesulfonic, isoethonic acids and the like and potassium carbonate sodium or potassium hydroxide ammonia, triethylamine, triethanolamine and the like.

Further aspects of the present invention will become apparent from the following description given by way of example only and with reference to the accompanying synthetic schemes.

### BRIEF DESCRIPTION OF DRAWINGS

FIG. 1

Illustrates the in vivo plasma concentrations of compound 3 over time following a single intraperitoneal administration (100 mg/kg) to C57Bl mice.

FIG. 2

Illustrates the growth curves for immunodeficient mice with NZM4 human tumour xenografts. Mice were either untreated (closed circles), treated with 2 Gray radiation alone (open circles) or treated with radiation combined with compound 3 (100 mg/kg per dose).

### DETAILED DESCRIPTION OF THE INVENTION

### Methods for Preparing Compounds of Formula I of the Invention

The 2-aryl-4-(amine)quinazolines can be synthesised by reaction of 2-aryl-4-chloroquinazolines with amines in a suitable solvent. The required 2-aryl-4-chloroquinazolines can be synthesised by chlorination of 2-arylquinazolinones. The required 2-arylquinazolinones can be conveniently synthesised by several different routes, depending on the substituents. Four suitable routes are:
1. Via boronic acid (Suzuki) coupling
2. Via amination of a substituted anthranilate ester and subsequent cyclisation
3. Via cyclisation of a substituted anthranilamide
4. Via reaction of 2-aminobenzamides with 2-(benzofuran-2-yl)acetyl halides

### Preparation of required 2-arylquinazolinone starting materials

The following examples are representative of the invention, and provide detailed methods for preparing the compounds of the invention. NMR spectra were obtained on a Bruker Avance-400 spectrometer at 400 MHz for ¹H and 100 MHz for ¹³C spectra, referenced to Me₄Si. Low resolution mass spectra were obtained on a Thermo Finnigan Surveyor MSQ. Column chromatography was carried out on silica gel, (Merck 230-400 mesh) unless otherwise stated.

### 1. Boronic Acid Route (Scheme 1):

Reaction of 2-cyanoanilines with carbon dioxide at ambient temperature in the presence of DBU gave 2-hydroxyquinazolinones (A) (T. Mizuno et al., Tett. Lett., 41, (2000), 1051). Chlorination of compounds (A) with POCl₃ and subsequent selective hydrolysis of the 4-chloro gave compounds (B) (J. De Ruiter et al., J. Med. Chem., 29(5), (1986), 627). Reaction of compounds (B) with aryl boronic acids in EtOH/toluene/water in the presence of catalytic amounts of PdCl₂(dppf gave 2-aryl-4-quinazolinones (C).

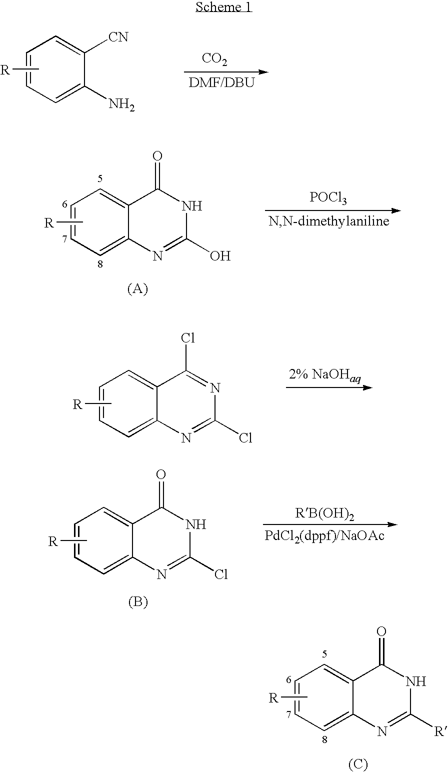

Preparation of 2-(6-methoxy-1-benzofuran-2-yl)-4(3H)-quinazolinone (example of general procedure). n-BuLi (6.0 mL, 2.5 M, 15 mmol) was added dropwise to a solution of 6-methoxy-1-benzofuran (2.002 g, 13.5 mmol) [M. Hideku et al., PCT Int. Appl. (2002) WO 2002100850] in THF (30 mL) at −78° C. over 5 min. The solution was stirred at −78° C. for 5 min. then triisopropylborate (15 mL, 65 mmol) was added and the mixture was warmed to room temperature. The mixture was quenched with HCl (2 M, 60 mL) and the organic solvent was removed in vacuo. Water (80 mL) and salt (10 g) were added and the mixture was cooled to 0° C. to give a white precipitate which was washed with water and hexanes to give 6-methoxy-1-benzofuran-2-ylboronic acid (1.408 g, 13.5 mmol). ¹H NMR (DMSO-d₆) δ ppm 8.37 (s, 2H), 7.53 (d, 1H, J=8.6 Hz), 7.37 (d, 1H, J=0.9 Hz), 7.12 (bd, 1H, J=1.7 Hz), 6.86 (dd, J=8.6, 2.2 Hz), 3.81 (s, 3H).

A mixture of the above 6-methoxy-1-benzofuran-2-ylboronic acid (1.30 g, 6.77 mmol), 2-chloro-4((3H)-quinazolinone (B: R═H) (1.067 g, 5.90 mmol), sodium acetate (2.30 g, 28 mmol) in ethanol (15 mL)/toluene (50 mL)/water (15 mL) was purged with nitrogen. PdCl₂(dppf (0.120 g, 0.147 mmol) was added and the mixture was purged with nitrogen then refluxed for 17 h. The mixture was cooled and the precipitate was filtered, dried and then columned (3:1 EtOAc:X4 to EtOAc) to give 2-(6-methoxy-1-benzofuran-2-yl)-4(3H)-quinazolinone (C: R═H, R′=6-methoxy-1-benzofuran-2-yl) (0.597 g, 34%) as a white solid. ¹H NMR (DMSO-d₆) δ ppm 11.5-13.0 (bs, 1H), 8.12 (dd, 1H, J=7.9, 1.2 Hz), 7.91 (s, 1H), 7.79 (td, 1H, J=7.0, 1.5 Hz), 7.71 (d, 1H, 7.8 Hz), 7.67 (d, 1H, J=8.6 Hz), 7.47 (td, 1H, J=7.5, 1.2 Hz), 7.33 (d, 1H, J=2.0 Hz), 6.97 (dd, 1H, J=8.6, 2.2 Hz), 3.86 (s, 3H). ACPI-MS Found: [M+H]⁺=293.

The following compounds were made using the above general procedure:

### Example 1.1

2-(2-Naphthyl)-4(3H)-quinazolinone (C: R═H, R′=2-naphthyl). Reaction of 2-chloro-4(3H)-quinazolinone (B: R═H) (0.400 g, 2.21 mmol) and 2-naphthaleneboronic acid (0.496 g, 2.88 mmol) using the general conditions gave the product (0.562 g, 93%) as an off white solid. ¹H NMR (DMSO-d₆) δ ppm 12.64 (bs, 1H), 8.83 (d, 1H, J=1.4 Hz), 8.33 (dd, 1H, J=8.6, 1.8 Hz), 8.17 (dd, 1H, J=7.9, 1.0 Hz), 7.98-8.10 (m, 3H), 7.74-7.86 (m, 2H), 7.59-7.69 (m, 2H), 7.52 (td, 1H, J=7.5, 1.3 Hz). ACPI-MS Found: [M+H]⁺=273.

### Example 1.2

2-(3-Quinolinyl)-4(3H)-quinazolinone (C: R═H, R′=3-quinolinyl). Reaction of 2-chloro-4(3H)-quinazolinone (B: R═H) (1.011 g, 5.56 mmol) and 3-quinolinylboronic acid (1.25 g, 7.23 mmol) using the general conditions gave the product (1.097 g, 71%) as a solid. ¹H NMR (DMSO-d₆) δ ppm 12.80 (bs, 1H), 9.62 (d, 1H, J=2.3 Hz), 9.16 (d, 1H, J=2.3 Hz), 8.20 (dd, 1H, J=8.0, 1.2 Hz), 8.10-8.15 (m, 2H), 7.80-7.91 (m, 3H), 7.73 (td, 1H, J=7.6, 1.0 Hz), 7.57 (ddd, 1H, J=7.8, 7.0, 1.3 Hz). ACPI-MS Found: [M+H]⁺=274.

### Example 1.3

2-(1-Benzothien-2-yl)-4(3H)-quinazolinone (C: R═H, R′=1-benzothien-2-yl). Reaction of 2-chloro-4(3H)-quinazolinone (B: R═H) (1.5 g, 8.3 mmol) and thianaphthene-2-boronic acid (2.21 g, 12.4 mmol) using the general conditions gave the product (1.288 g, 56%) as a solid. ¹H NMR (DMSO-d₆) δ ppm 12.84 (bs, 1H), 8.58 (bs, 1H), 8.16 (dd, 1H, J=7.9, 1.2 Hz), 8.04 (d, 1H, J=7.8 Hz), 7.94 (dd, 1H, 7.0, 1.2 Hz), 7.85 (ddd, 1H, J=8.1, 7.2, 1.5 Hz), 7.71 (d, 1H, J=7.7 Hz), 7.43-7.57 (m, 3H). ACPI-MS Found: [M+H]⁺=279.

### Example 1.4

2-(5-Methoxy-1-benzofuran-2-yl)-4(3H)-quinazolinone (C: R═H, R′=5-methoxy-1-benzofuran-2-yl) Reaction of 2-chloro-4(3H)-quinazolinone (B: R═H) (0.290 g, 1.60 mmol) and 5-methoxy-1-benzofuran-2-ylboronic acid (0.460 g, 2.39 mmol) using the general conditions gave the product (0.342 g, 73%) as a solid. ¹H NMR (DMSO-d₆) δ ppm 12.71 (s, 1H), 8.16 (dd, 1H, J=7.9, 1.2 Hz), 8.01 (s, 1H), 7.86 (ddd, 1H, J=8.1, 7.2, 1.5 Hz), 7.77 (dd, 1H, J=8.1, 0.7 Hz), 7.65 (d, 1H, J=9.0 Hz), 7.55 (td, 1H, J=7.5, 1.1 Hz), 7.32 (d, 1H, J=2.6 Hz), 7.08 (dd, 1H, J=9.0, 2.6 Hz), 3.83 (s, 3H). ACPI-MS Found: [M+H]⁺=293.

### 2. Amide Route (Scheme 2):

The acid chlorides (R′COCl) required for this method can be prepared in various ways. Benzo[b]furan-2-carbonyl chloride was synthesised by refluxing benzo[b]furan-2-carboxylic acid in thionyl chloride for 15 min, then removing the excess thionyl chloride in vacuo. In the case of indole-2-carbonyl chlorides, PCl₅ (1.1 equiv.) was added to a slurry of the indole-2-carboxylic acid (1.0 equiv.) in ether (0.1 mol acid to 400 mL ether). After 16 h the solvent was removed in vacuo, ether was added and removed in vacuo (repeated twice) and this procedure was performed using chloroform to give the indole-2-carbonyl chloride which was used in the coupling step.

A solution of the acid chloride (1.05-1.1 equiv.) and anthranilamide (D) (1 eq) in pyridine with a catalytic amount of 4-dimethylaminopyridine was refluxed for a specified time. The solution was poured onto crushed ice and the resultant precipitate was filtered. The crude intermediate amide (E) was then refluxed in a solution of 5% aqueous KOH and ethanol (2:1 mixture) for a specified time (generally 0.5 h), cooled and acidified with 2 M hydrochloric acid or glacial acetic acid to precipitate the quinazolinone (C). The amide formation and subsequent cyclisation were monitored by GCMS.

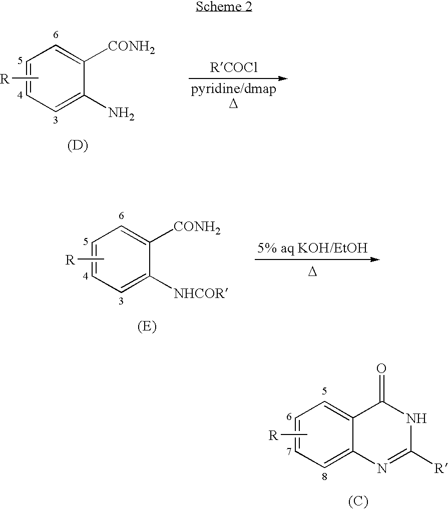

### Example 2.1

2-(1-Benzofuran-2-yl)-4(3H)-quinazolinone (C: R═H, R′=1-benzofuran-2-yl). The intermediate amide (E: R═H, R′═H) was synthesised by refluxing anthranilamide (2.22 g, 16.3 mmol) and 1-benzofuran-2-carbonyl chloride (from benzo[b]furan-2-carboxylic acid, 2.79 g, 17.2 mmol) in pyridine (30 mL) for 0.5 h. The intermediate amide was refluxed in 5% aqueous KOH (40 mL)/EtOH (20 mL) for 0.5 h to give the product (3.56 g, 83%) as a solid. ¹H NMR (DMSO-d₆) δ ppm 12.75 (bs, 1H), 8.17 (dd, 1H, J=7.9, 1.2 Hz), 8.08 (d, 1H, J=0.7 Hz), 7.73-7.89 (m, 4H), 7.56 (td, 1H, J=7.3, 1.2 Hz), 7.50 (ddd, 1H, J=8.3, 7.5, 1.2 Hz), 7.37 (td, 1H, J=7.5, 0.9 Hz). ACPI-MS Found: [M+H]⁺=263.

### Example 2.2

2-(1-Benzofuran-2-yl)-5-chloro-4(3H)-quinazolinone (C: R=5-Cl, R′=1-benzofuran-2-yl). The intermediate amide (E: R=6-Cl, R′=1-benzofuran-2-yl) was synthesised by refluxing 2-amino-6-chlorobenzamide (0.528 g, 3.10 mmol) (S. W. Schneller et al., J. Med. Chem., 32(10), (1989), 2247) and 1-benzofuran-2-carbonyl chloride (0.615 g, 3.41 mmol) in pyridine (20 mL) for 0.5 h. The intermediate amide was refluxed in 5% aqueous KOH (40 mL)/EtOH (20 mL) for 0.5 h to give the product, which was used in subsequent steps without purification.

### Example 2.3

2-(1-Benzofuran-2-yl)-6-methyl-4(3H)-quinazolinone (C: R=6-Me, R′=1-benzofuran-2-yl). The intermediate amide (E: R=5-Me, R′=1-benzofuran-2-yl) was synthesised by refluxing 2-amino-5-methylbenzamide (0.357 g, 2.38 mmol) and 1-benzofuran-2-carbonyl chloride (from benzo[b]furan-2-carboxylic acid, 0.426 g, 2.62 mmol) in pyridine (30 mL) for 0.5 h. The intermediate amide was refluxed in 5% aqueous KOH (40 mL)/EtOH (20 mL) for 0.5 h to give the product (0.239 g, 61%). ¹H NMR (DMSO-d₆) δ ppm 12.66 (bs, 1H), 8.04 (d, 1H, J=0.7 Hz), 7.97 (bs, 1H), 7.82 (bd, 1H, J=7.6 Hz), 7.74 (dd, 1H, J=8.4, 0.7 Hz), 7.65-7.71 (m, 2H), 7.49 (btd, 1H, J=8.3, 7.5, 1.3 Hz), 7.36 (td, 1H, J=7.5, 0.8 Hz), 2.47 (s, 3H). ACPI-MS Found: [M+H]⁺=277.

### Example 2.4

2-(1-Benzofuran-2-yl)-6-(trifluoromethyl)-4(3H)-quinazolinone (C: R=6-CF₃, R′=1-benzofuran-2-yl). The intermediate amide (E: R=5-CF₃, R′=1-benzofuran-2-yl) was synthesised by refluxing 2-amino-5-(trifluoromethyl)benzamide (0.115 g, 0.563 mmol) and 1-benzofuran-2-carbonyl chloride (from benzo[b]furan-2-carboxylic acid, 0.100 g, 0.617 mmol) in pyridine (10 mL) for 1 h. The intermediate amide was refluxed in 5% aqueous KOH (10 mL)/EtOH (5 mL) for 0.5 h to give the product (0.136 g, 73%) as a solid. ¹H NMR (DMSO-d₆) δ ppm 13.09 (bs, 1H), 8.39 (dd, 1H, J=1.6, 0.4 Hz), 8.11-8.16 (m, 2H), 7.97 (d, 1H, J=8.6 Hz), 7.85 (d, 1H, J=7.5 Hz), 7.76 (dd, 1H, J=8.4, 0.7 Hz), 7.52 (ddd, 1H, J=8.3, 7.3, 1.3 Hz), 7.38 (td, 1H, J=7.5, 0.9 Hz). ACPI-MS Found: [M+H]⁺=331.

### Example 2.5

2-(1-Benzofuran-2-yl)-6-fluoro-4(3H)-quinazolinone (C: R=6-F, R′=1-benzofuran-2-yl). The intermediate amide (E: R=5-F, R′=1-benzofuran-2-yl) was synthesised by refluxing 2-amino-5-fluorobenzamide (0.241 g, 1.56 mmol) and 1-benzofuran-2-carbonyl chloride (0.350 g, 1.93 mmol) in pyridine (10 mL) for 0.5 h. The intermediate amide was refluxed in 5% aqueous KOH (30 mL)/EtOH (15 mL) for 1 h to give the product (0.422 g, 96%) as a solid. ¹H NMR (DMSO-d₆) δ ppm 10.8 (bs, 1H), 8.06 (d, 1H, J=0.7 Hz), 7.80-7.89 (m, 3H), 7.71-7.77 (m, 2H), 7.50 (td, 1H, J=7.2, 1.3 Hz), 7.37 (td, 1H, J=7.5, 0.9 Hz). ACPI-MS Found: [M+H]⁺=281.

### Example 2.6

2-(1-Benzofuran-2-yl)-6-chloro-4(3H)-quinazolinone (C: R=6-Cl, R′=1-benzofuran-2-yl). The intermediate amide (E: R=5-Cl, R′=1-benzofuran-2-yl) was synthesised by refluxing 2-amino-5-chlorobenzamide (0.552 g, 3.24 mmol) and 1-benzofuran-2-carbonyl chloride (0.640 g, 3.54 mmol) in pyridine (20 mL) for 1 h. The intermediate amide was refluxed in 5% aqueous KOH (20 mL)/EtOH (10 mL) for 0.5 h to give the product (0.771 g, 80%) as a solid. ¹H NMR (DMSO-d₆) δ ppm 12.92 (bs, 1H), 8.06-8.11 (m, 2H), 7.89 (dd, 1H, J=8.7, 2.5 Hz), 7.79-7.85 (m, 2H), 7.75 (dd, 1H, J=8.4, 0.7 Hz), 7.51 (ddd, 1H, J=8.3, 7.3, 1.3 Hz), 7.37 (td, 1H, J=7.5, 0.9 Hz). ACPI-MS Found: [M+H]⁺=297, 299.

### Example 2.7

2-(1-Benzofuran-2-yl)-6-bromo-4(3H)-quinazolinone (C: R=6-Br, R′=1-benzofuran-2-yl). The intermediate amide (E: R=5-Br, R′=1-benzofuran-2-yl) was synthesised by refluxing 2-amino-5-bromobenzamide (0.800 g, 3.72 mmol) (M. Tobe et al, Bioorg. Med. Chem., 11(3), (2003), 383) and 1-benzofuran-2-carbonyl chloride (0.740 g, 4.10 mmol) in pyridine (40 mL) for 1 h. The intermediate amide was refluxed in 5% aqueous KOH (40 mL)/EtOH (20 mL) for 0.5 h to give the product (1.066 g, 84%) as a solid. ¹H NMR (DMSO-d₆) δ ppm 12.93 (bs, 1H), 8.24 (d, 1H, J=2.4 Hz), 8.08 (s, 1H), 7.99 (dd, 1H, J=8.7, 2.4 Hz), 7.83 (d, 1H, J=7.6 Hz), 7.71-7.77 (m, 2H), 7.51 (ddd, 1H, J=8.4, 7.3, 1.3 Hz), 7.37 (td, 1H, J=7.5, 0.8 Hz). ACPI-MS Found: [M+H]⁺=343, 341.

### Example 2.8

2-(1-Benzofuran-2-yl)-6-nitro-4(3H)-quinazolinone (C: R=6-NO₂, R′=1-benzofuran-2-yl). The intermediate amide (E: R=5-NO₂, R′=1-benzofuran-2-yl) was synthesised by refluxing 2-amino-5-nitrobenzamide (1.572 g, 8.68 mmol) and 1-benzofuran-2-carbonyl chloride (from benzo[b]furan-2-carboxylic acid, 1.520 g, 9.37 mmol) in pyridine (40 mL) for 0.5 h. The intermediate amide was refluxed in 5% aqueous KOH (50 mL)/EtOH (25 mL) for 0.5 h to give the product (2.05 g, 77%) as a solid. ¹H NMR (DMSO-d₆) δ ppm 13.22 (bs, 1H), 8.83 (d, 1H, J=2.9 Hz), 8.55 (dd, 1H, J=9.0, 2.7 Hz), 8.16 (s, 1H), 7.94 (d, 1H, J=9.0 Hz), 7.85 (d, 1H, J=7.7 Hz), 7.76 (dd, 1H, J=8.4, 0.5 Hz), 7.53 (td, 1H, J=7.5, 1.2 Hz), 7.39 (td, 1H, J=7.5, 0.7 Hz). ACPI-MS Found: [M+H]⁺=308.

### Example 2.9

2-(1-Benzofuran-2-yl)-4-oxo-3,4-dihydro-6-quinazolinecarboxamide (C: R=6-CONH₂, R′=1-benzofuran-2-yl). The intermediate amide (E: R=5-CONH₂, R′=1-benzofuran-2-yl) was synthesised by refluxing 4-aminoisophthalamide (0.450 g, 2.51 mmol) (Y. Takase et al, J. Med. Chem., 37(13), (1994), 2106) and 1-benzofuran-2-carbonyl chloride (0.500 g, 2.77 mmol) in pyridine (30 mL) for 1 h. The intermediate amide was refluxed in 5% aqueous KOH (30 mL)/EtOH (15 mL) for 1 h to give the product (0.600 g, 78%) as a solid. ¹H NMR (DMSO-d₆) δ ppm 12.91 (bs, 1H), 8.70 (d, 1H, J=2.0 Hz), 8.20-8.32 (m, 2H), 8.11 (d, 1H, J=0.7 Hz), 7.80-7.87 (m, 2H), 7.76 (dd, 1H, J=8.7, 0.7 Hz), 7.45-7.54 (m, 2H), 7.37 (td, 1H, J=7.5, 0.7 Hz). ACPI-MS Found: [M+H]⁺=306.

### Example 2.10

2-(1-Benzofuran-2-yl)pyrido[3,4-d]pyrimidin-4(3H)-one (C: R=7-aza, R′=1-benzofuran-2-yl). A slurry of 3-aminoisonicotinic acid (1.556 g, 11.3 mmol) and CDl (2.82 g, 17.4 mmol) in dmf (20 mL) was heated to 40° C. for 0.5 h then cooled. Concentrated aqueous ammonia (50 mL) was added and the mixture was stirred for 15 min then extracted with ethyl acetate. Removal of the solvent gave a solid which was dissolved in pyridine (20 mL), 1-benzofuran-2-carbonyl chloride (from benzo[b]furan-2-carboxylic acid; 2.006 g, 12.4 mmol) was added and the mixture was refluxed for 0.5 h, poured onto ice and filtered to give the intermediate amide (E: R=4-aza, R′=1-benzofuran-2-yl). The intermediate amide was refluxed in 5% aqueous KOH (20 mL)/EtOH (10 mL) for 0.5 h to give the product (0.303 g, 10%) as a solid. ¹H NMR (DMSO-d₆) δ ppm 13.07 (bs, 1H), 9.15 (d, 1H, J=0.8 Hz), 8.68 (d, 1H, J=5.1 Hz), 8.11 (d, 1H, J=0.8 Hz), 7.98 (dd, 1H, J=5.1, 0.8 Hz), 7.84 (dd, 1H, J=7.5, 0.8 Hz), 7.77 (dd, 1H, J=8.3, 0.8 Hz), 7.51 (ddd, 1H, J=8.3, 7.3, 1.2 Hz), 7.38 (td, 1H, J=7.5, 0.8 Hz). ACPI-MS Found: [M+H]⁺=264.

### Example 2.11

2-(1-Benzofuran-2-yl)-7-methyl-4(3H)-quinazolinone (C: R=7-CH₃, R′=1-benzofuran-2-yl). The intermediate amide (E: R=4-CH₃, R′=1-benzofuran-2-yl) was synthesised by refluxing 2-amino-4-methylbenzamide (0.380 g, 2.53 mmol) and 1-benzofuran-2-carbonyl chloride (0.503 g, 2.79 mmol) in pyridine (20 mL) for 1 h. The intermediate amide was refluxed in 5% aqueous KOH (20 mL)/EtOH (10 mL) for 1 h to give the product (0.558 g, 80%) as a solid. ¹H NMR (DMSO-d₆) δ ppm 12.64 (bs, 1H), 8.02-8.07 (m, 2H), 7.81 (d, 1H, J=7.6 Hz), 7.74 (dd, 1H, J=8.4, 0.6 Hz), 7.60 (s, 1H), 7.49 (ddd, 1H, J=8.4, 7.5, 1.2 Hz), 7.33-7.39 (m, 2H), 2.50 (s, 3H). ACPI-MS Found: [M+H]⁺=277.

### Example 2.12

2-(1-Benzofuran-2-yl)-7-fluoro-4(3H)-quinazolinone (C: R=7-F, R′=1-benzofuran-2-yl). The intermediate amide (E: R=4-F, R′=1-benzofuran-2-yl) was synthesised by refluxing 2-amino-4-fluorobenzamide (0.300 g, 1.94 mmol) and 1-benzofuran-2-carbonyl chloride (0.420 g, 2.33 mmol) in pyridine (10 mL) for 0.5 h. The intermediate amide was refluxed in 5% aqueous KOH (30 mL)/EtOH (150 mL) for 1 h to give the product (0.505 g, 93%) as a solid. ¹H NMR (DMSO-d₆) δ ppm 12.85 (s, 1H), 8.22 (dd, 1H, J=8.8, 6.3 Hz), 8.10 (d, 1H, J=0.7 Hz), 7.84 (d, 1H, J=7.6 Hz), 7.76 (dd, 1H, J=8.4, 0.7 Hz), 7.58 (dd, 1H, J=10.1, 2.5 Hz), 7.51 (td, 1H, J=7.3, 1.3 Hz), 7.35-7.44 (m, 2H). ACPI-MS Found: [M+H]⁺=281.

### Example 2.13

2(1-Benzofuran-2-yl)-7-chloro-4(3H)-quinazolinone (C: R=7-Cl, R′=1-benzofuran-2-yl). The intermediate amide (E: R=4-Cl, R′=1-benzofuran-2-yl) was synthesised by refluxing 2-amino-4-chlorobenzamide (0.417 g, 2.76 mmol) (B. O. Javier et al., PCT Int Appl. 2001066519) and 1-benzofuran-2-carbonyl chloride (0.550 g, 3.05 mmol) in pyridine (20 mL) for 1 h. The intermediate amide was refluxed in 5% aqueous KOH (20 mL)/EtOH (10 mL) for 0.5 h to give the product (0.490 g, 60%) as a solid. ¹H NMR (DMSO-d₆) δ ppm 12.86 (s, 1H), 8.15 (d, 1H, J=8.5 Hz), 8.08 (d, 1H, J=0.7 Hz), 7.80-7.86 (m, 2H), 7.75 (dd, 1H, J=8.4, 0.7 Hz), 7.56 (dd, 1H, J=8.5, 2.1 Hz), 7.51 (ddd, 1H, J=8.3, 7.3, 1.3 Hz), 7.37 (td, 1H, J=7.5, 0.9 Hz). ACPI-MS Found: [M+H]⁺=297, 299.

### Example 2.14

2-(1-Benzofuran-2-yl)-7-bromo-4(3H)-quinazolinone (C: R=7-Br, R′=1-benzofuran-2-yl). The intermediate amide (E: R=4-Br, R′=1-benzofuran-2-yl) was synthesised by refluxing 2-amino-4-bromobenzamide (0.424 g, 1.97 mmol) (V. Joshi et al., Ind. J. Chem. Sec. B, 26(1-12), (1987), 602) and 1-benzofuran-2-carbonyl chloride (0.430 g, 2.38 mmol) in pyridine (20 mL) for 0.5 h. The intermediate amide was refluxed in 5% aqueous KOH (60 mL)/EtOH (30 mL) for 1 h to give the product (0.640 g, 79%) as a solid. ¹H NMR (DMSO-d₆) δ ppm 12.86 (bs, 1H), 8.04-8.08 (m, 2H), 7.98 (d, 1H, J=1.9 Hz), 7.83 (d, 1H, J=7.6 Hz), 7.75 (dd, 1H, J=8.3, 0.6 Hz), 7.69 (dd, 1H, J=8.5, 1.9 Hz), 7.50 (ddd, 1H, J=8.4, 7.3, 1.2 Hz), 7.37 (td, 1H, J=7.5, 0.8 Hz). ACPI-MS Found: [M+H]⁺=343, 341.

### Example 2.15

2-(1-Benzofuran-2-yl)-7-nitro-4(3H)-quinazolinone (C: R=7-NO₂, R′=1-benzofuran-2-yl). The intermediate amide (E: R=4-NO₂, R′=1-benzofuran-2-yl) was synthesised by refluxing 2-amino-4-nitrobenzamide (0.406 g, 2.24 mmol) and 1-benzofuran-2-carbonyl chloride (from benzo[b]furan-2-carboxylic acid, 0.400 g, 2.47 mmol) in pyridine (10 mL) for 1 h. The intermediate amide was refluxed in 5% aqueous KOH (20 mL)/EtOH (10 mL) for 0.5 h to give the product (0.511 g, 74%) as a solid. ¹H NMR (DMSO-d₆) δ ppm 13.13 (bs, 1H), 8.46 (d, 1H, J=2.2 Hz), 8.37 (d, 1H, J=8.7 Hz), 8.23 (dd, 1H, J=8.8, 2.2 Hz), 8.14 (d, 1H, J=0.7 Hz), 7.85 (d, 1H, J=7.6 Hz), 7.76 (dd, 1H, J=8.4, 0.6 Hz), 7.52 (ddd, 1H, J=8.3, 7.2, 1.2 Hz), 7.38 (td, 1H, J=7.5, 0.8 Hz). ACPI-MS Found: [M+H]⁺=308.

### Example 2.16

2-(1-Benzofuran-2-yl)-8-methyl-4(3H)-quinazolinone (C: R=8-Me, R′=1-benzofuran-2-yl). The intermediate amide (E: R=3-Me, R′=1-benzofuran-2-yl) was synthesised by refluxing 2-amino-3-methylbenzamide (0.359 g, 2.39 mmol) and 1-benzofuran-2-carbonyl chloride (from benzo[b]furan-2-carboxylic acid, 0.426 g, 2.63 mmol) in pyridine (30 mL) for 3 h. The intermediate amide was refluxed in 5% aqueous KOH (40 mL)/EtOH (20 mL) for 2 h to give the product (0.433 g, 66%) as a solid. ¹H NMR (DMSO-d₆) δ ppm 12.70 (bs, 1H), 8.05 (d, 1H, J=0.8 Hz), 8.01 (dd, 1H, J=7.9, 0.9 Hz), 7.83 (bd, 1H, J=7.5 Hz), 7.76 (dd, 1H, J=8.3, 0.7 Hz), 7.72 (m, 1H), 7.49 (ddd, 1H, J=8.3, 7.5, 1.3 Hz), 7.43 (t, 1H, J=7.6 Hz), 7.37 (td, 1H, J=7.5, 0.8 Hz), 2.65 (s, 3H). ACPI-MS Found: [M+H]⁺=277.

### Example 2.17

2-(1-Benzofuran-2-yl)-8-methoxy-4(3H)-quinazolinone (C: R=8-OMe, R′=1-benzofuran-2-yl. The intermediate amide (E: R=3-OMe, R′=1-benzofuran-2-yl) was synthesised by refluxing 2-amino-3-methoxybenzamide (0.480 g, 2.89 mmol) [R. J. Griffin et al., J. Med. Chem., 1988, 41, 5247] and 1-benzofuran-2-carbonyl chloride (from benzo[b]furan-2-carboxylic acid, 0.520 g, 3.21 mmol) in pyridine (40 mL) for 2 h. The intermediate amide was refluxed in 5% aqueous KOH (20 mL)/EtOH (10 mL) for 0.5 h to give the product (0.203 g, 24%) as a solid. ¹H NMR (DMSO-d₆) δ ppm 12.71 (bs, 1H), 8.02 (s, 1H), 7.82 (bd, 1H, J=7.6 Hz), 7.76 (dd, 1H, J=8.3, 0.6 Hz), 7.71 (dd, 1H, J=7.8, 1.4 Hz), 7.44-7.52 (m, 2H), 7.33-7.42 (m, 2H), 3.97 (s, 3H). ACPI-MS Found: [M+H]⁺=293.

### Example 2.18

2-(1-Benzofuran-2-yl)-8-chloro-4(3H)-quinazolinone (C: R=8-Cl, R′=1-benzofuran-2-yl). The intermediate amide (E: R=3-Cl, R′=1-benzofuran-2-yl) was synthesised by refluxing 2-amino-3-chlorobenzamide (0.168 g, 0.985 mmol) [R. C. Andrews et al., U.K. Patent Appl. 1996, GB 2295387] and 1-benzofuran-2-carbonyl chloride (0.205 g, 1.14 mmol) in pyridine (10 mL) for 0.5 h. The intermediate amide was refluxed in 5% aqueous KOH (10 mL)/EtOH (5 mL) for 0.5 h to give the product (49 mg, 17%) as a solid. ¹H NMR (DMSO-d₆) δ ppm 12.96 (bs, 1H), 8.10 (dd, 1H, J=7.9, 1.3 Hz), 8.08 (s, 1H), 8.00 (dd, 1H, J=7.7, 1.3 Hz), 7.84 (d, 1H, J=7.7 Hz), 7.77 (d, 1H, J=8.4 Hz), 7.45-7.54 (m, 2H), 7.38 (t, 1H, J=7.3 Hz). ACPI-MS Found: [M+H]⁺=297, 299.

### Example 2.19

2-(1-Benzofuran-2-yl)-4-oxo-3,4-dihydro-8-quinazolinecarboxamide (C: R=8-CONH₂, R′=1-benzofuran-2-yl). The intermediate amide (E: R=3-CONH₂, R′=1-benzofuran-2-yl) was synthesised by refluxing 3-aminophthalamide (0.410 g, 2.29 mmol) and 1-benzofuran-2-carbonyl chloride (0.455 g, 2.52 mmol) in pyridine (20 mL) for 1 h. The intermediate amide was refluxed in 5% aqueous KOH (20 mL)/EtOH (10 mL) for 1 h to give the product as a solid, the crude material was used in the subsequent step. ACPI-MS Found: [M+H]⁺=306.

### Example 2.20

2-(1-Benzofuran-2-yl)-6,7-dichloro-4(3H)-quinazolinone (C: R=6,7-diCl, R′=1-benzofuran-2-yl). 4,5-Dichloro-2-nitrobenzoic acid (1.665 g, 7.05 mmol) was refluxed in thionyl chloride (25 mL) for 10 min. The solvent was removed in vacuo and the residue was dissolved in thf (20 mL), ammonia gas was bubbled through the solution until conversion to the amide was complete. The solvent was removed in vacuo and the residue was partitioned between EtOAc/water, removal of the solvent from the organic layer gave 4,5-dichloro-2-nitrobenzamide (1.60 g, 97%). ¹H NMR (DMSO-d₆) δ ppm 8.37 (s, 1H), 8.21 (bs, 1H), 7.97 (s, 1H), 7.84 (bs, 1H). ACPI-MS Found: [M+H]⁺=236.

Iron dust (0.40 g, 7.1 mmol) was added to a solution of 4,5-dichloro-2-nitrobenzamide (0.150 g, 0.638 mmol) in EtOH/water (4:1, 20 mL) and acetic acid (0.4 mL) at reflux. After 10 min. the mixture was cooled and aqueous ammonia was added, the mixture was filtered through celite and the solvent was removed in vacuo. The residue was partitioned between DCM/water, removal of the solvent from the organic layer gave 2-amino-4,5-dichlorobenzamide (62 mg, 47%). ¹H NMR (DMSO-d₆) δ ppm 7.88 (bs, 1H), 7.77 (s, 1H), 7.24 (bs, 1H), 6.93 (s, 1H), 6.85 (bs, 2H). ACPI-MS Found: [M+H]⁺=206.

The intermediate amide (E: R=4,5-diCl, R′=1-benzofuran-2-yl) was synthesised by refluxing 2-amino-4,5-dichlorobenzamide (0.062 g, 0.30 mmol) and 1-benzofuran-2-carbonyl chloride (0.060 g, 0.33 mmol) in pyridine (5 mL) for 0.5 h. The intermediate amide was refluxed in 5% aqueous KOH (10 mL)/EtOH (5 mL) for 0.5 h to give the product (99 mg g, 99%) as a solid. ¹H NMR (DMSO-d₆) δ ppm 8.24 (s, 1H), 8.06 (bs, 2H), 7.83 (bd, 1H, J=7.5 Hz), 7.76 (dd, 1H, J=8.4, 0.7 Hz), 7.50 (ddd, 1H, J=8.3, 7.3, 1.2 Hz), 7.37 (td, 1H, J=7.5, 0.9 Hz), 6.5-9.0 (b, 1H). ACPI-MS Found: [M+H]⁺=331, 333, 335.

### Example 2.21

2-(1-Benzofuran-2-yl)-7,8-dimethoxy-4(3H)-quinazolinone (C: R=7,8-diOMe, R′=1-benzofuran-2-yl). The intermediate amide (E: R=3,4-diOMe, R′=1-benzofuran-2-yl) was synthesised by refluxing 2-amino-3,4-dimethoxybenzamide (0.241 g, 1.23 mmol) (J. Maillard et al., Chim. Ther., 2(4), (1967), 231) and 1-benzofuran-2-carbonyl chloride (0.209 g, 1.29 mmol) in pyridine (15 mL) for 2 h. The intermediate amide was refluxed in 5% aqueous KOH (20 mL)/EtOH (10 mL) for 1 h to give the product, which was used without further purification.

### Example 2.22

2-(3-Methyl-1-benzofuran-2-yl)-4(3H)-quinazolinone (C: R═H, R′=3-methyl-1-benzofuran-2-yl). The intermediate amide (E: R═H, R′=3-methyl-1-benzofuran-2-yl) was synthesised by refluxing 2-aminobenzamide (1.10 g, 8.08 mmol) and 3-methyl-1-benzofuran-2-carbonyl chloride (from 3-methyl-1-benzofuran-2-carboxylic acid, 1.50 g, 8.51 mmol) in pyridine (50 mL) for 0.5 h. The intermediate amide was refluxed in 5% aqueous KOH (100 mL)/EtOH (50 mL) for 1 h to give the product (1.988 g, 89%) as a solid. ¹H NMR (DMSO-d⁶) δ ppm 12.42 (bs, 1H), 8.15 (dd, 1H, J=7.9, 1.2 Hz), 7.78-7.87 (m, 2H), 7.73 (d, 1H, J=7.5 Hz), 7.64 (d, 1H, J=8.3 Hz), 7.47-7.56 (m, 2H), 7.38 (td, 1H, J=7.5, 0.9 Hz), 2.75 (s, 3H). ACPI-MS Found: [M+H]⁺=277.

### Example 2.23

8-Methyl-2-(3-methyl-1-benzofuran-2-yl)-4(3H)-quinazolinone (C: R=8-Me, R′=3-methyl-1-benzofuran-2-yl). The intermediate amide (E: R=3-Me, R′=3-methyl-1-benzofuran-2-yl) was synthesised by refluxing 2-amino-3-methylbenzamide (0.500 g, 3.32 mmol) and 3-methyl-1-benzofuran-2-carbonyl chloride (from 3-methyl-1-benzofuran-2-carboxylic acid (0.643 g, 3.64 mmol) in pyridine (20 mL) for 0.5 h. The intermediate amide was refluxed in 5% aqueous KOH (20 mL)/EtOH (10 mL) for 1 h to give the product (0.720 g, 75%) as a solid. ¹H NMR (DMSO-d₆) δ ppm 12.2 (bs, 1H), 7.99 (dd, 1H, J=7.9, 0.8 Hz), 7.81 (d, 1H, J=7.5 Hz), 7.72 (dq, 1H, J=7.3, 0.6 Hz), 7.64 (d, 1H, J=8.3 Hz), 7.50 (ddd, 1H, J=8.3, 7.2, 1.3 Hz), 7.36-7.43 (m, 2H), 2.77 (s, 3H), 2.62 (s, 3H). ACPI-MS Found: [M+H]⁺=291.

### Example 2.24

2-(5-Methyl-1-benzofuran-2-yl)-4(3H)-quinazolinone (C: R═H, R′=5-methyl-1-benzofuran-2-yl). The intermediate amide (E: R═H, R′=5-methyl-1-benzofuran-2-yl) was synthesised by refluxing 2-aminobenzamide (0.387 g, 2.84 mmol) and 5-methyl-1-benzofuran-2-carbonyl chloride (from 5-methyl-1-benzofuran-2-carboxylic acid, 0.527 g, 2.99 mmol (C. B. Chapleo, J. Med. Chem., 27(5), (1984), 570)) in pyridine (10 mL) for 1 h. The intermediate amide was refluxed in 5% aqueous KOH (20 mL)/EtOH (10 mL) for 0.5 h to give the product (0.735 g, 94%) as a solid. ¹H NMR (DMSO-d₆) δ ppm 12.69 (bs, 1H), 8.16 (dd, 1H, J=7.9, 1.2 Hz), 7.99 (d, 1H, J=0.8 Hz), 7.85 (ddd, 1H, J=8.3, 7.1, 1.5 Hz), 7.77 (dd, 1H, J=8.1, 0.7 Hz), 7.58-7.65 (m, 2H), 7.54 (ddd, 1H, J=8.1, 7.1, 1.1 Hz), 7.31 (dd, 1H, J=8.5, 1.4 Hz), 2.43 (s, 3H). ACPI-MS Found: [M+H]⁺=277.

### Example 2.25

2-(5-Chloro-1-benzofuran-2-yl)-4(3H)-quinazolinone (C: R═H, R′=5-chloro-1-benzofuran-2-yl). The intermediate amide (E: R═H, R′=5-chloro-1-benzofuran-2-yl) was synthesised by refluxing 2-aminobenzamide (0.102 g, 0.749 mmol) and 5-chloro-1-benzofuran-2-carbonyl chloride (from 5-chloro-1-benzofuran-2-carboxylic acid, 0.155 g, 0.788 mmol) in pyridine (10 mL) for 1 h. The intermediate amide was refluxed in 5% aqueous KOH (10 mL)/EtOH (5 mL) for 1 h to give the product (0.140 g, 63%) as a solid. ¹H NMR (DMSO-d₆) δ ppm 12.79 (bs, 1H), 8.16 (dd, 1H, J=7.9, 1.2 Hz), 8.03 (d, 1H, J=0.8 Hz), 7.93 (d, 1H, J=2.0 Hz), 7.86 (ddd, 1H, J=8.3, 7.1, 1.5 Hz), 7.76-7.81 (m, 2H), 7.56 (ddd, 1H, J=8.1, 7.1, 1.2 Hz), 7.51 (dd, 1H, J=8.8, 2.2 Hz). ACPI-MS Found: [M+H]⁺=299, 297.

### Example 2.26

245-Bromo-1-benzofuran-2-yl)-4(3H)-quinazolinone (C: R═H, R′=5-bromo-1-benzofuran-2-yl). The intermediate amide (E: R═H, R′=5-bromo-1-benzofuran-2-yl) was synthesised by refluxing 2-aminobenzamide (0.310 g, 2.28 mmol) and 5-bromo-1-benzofuran-2-carbonyl chloride (from 5-bromo-1-benzofuran-2-carboxylic acid, 0.577 g, 2.39 mmol) in pyridine (20 mL) for 1 h. The intermediate amide was refluxed in 5% aqueous KOH (40 mL)/EtOH (20 mL) for 1 h to give the product (0.648 g, 78%) as a solid. ¹H NMR (DMSO-d₆) δ ppm 12.5 (b, 1H), 8.14 (dd, 1H, J=7.9, 1.2 Hz), 8.06 (d, 1H, J=1.9 Hz), 7.94 (d, 1H, J=0.6 Hz), 7.81 (ddd, 1H, J=8.3, 7.1, 1.5 Hz), 7.70-7.76 (m, 2H), 7.60 (dd, 1H, J=8.8, 2.1 Hz), 7.51 (ddd, 1H, J=8.1, 7.1, 1.2 Hz). ACPI-MS Found: [M+H]⁺=343, 341.

### Example 2.27

2-(5-Methoxy-1H-indol-2-yl)-4(3H)-quinazolinone (C: R═H, R′=5-methoxy-1H-indol-2-yl). The intermediate amide (E: R═H, R′=5-methoxy-1H-indol-2-yl) was synthesised by refluxing 2-aminobenzamide (3.343 g, 24.6 mmol) and 5-methoxy-1H-indole-2-carbonyl chloride (from 5-methoxy-1H-indole-2-carboxylic acid; 4.99 g, 26.1 mmol) in pyridine (100 mL) for 0.5 h. The intermediate amide was refluxed in 5% aqueous KOH (200 mL)/EtOH (100 mL) for 15 min to give the product (6.25 g, 87%) as a solid. ¹H NMR (DMSO-d₆) δ ppm 12.53 (bs, 1H), 11.62 (s, 1H), 8.14 (dd, 1H, J=7.9, 1.2 Hz), 7.84 (td, 1H, J=7.6, 1.5 Hz), 7.71 (d, 1H, J=7.7 Hz), 7.58 (d, 1H, J=1.5 Hz), 7.49 (td, 1H, J=7.5, 1.0 Hz), 7.42 (d, 1H, J=8.9 Hz), 7.11 (d, 1H, J=2.3 Hz), 6.89 (dd, 1H, J=8.9, 2.5 Hz), 3.78 (s, 3H). ACPI-MS Found: [M+H]⁺=292.

### Example 2.28

2-(5-Methoxy-1-methyl-1H-indol-2-yl).4(3H)-quinazolinone (C: R═H, R′=5-methoxy-1-methyl-1H-indol-2-yl). The intermediate amide (E: R═H, R′=5-methoxy-1-methyl-1H-indol-2-yl) was synthesised by refluxing 2-aminobenzamide (0.329 g, 2.42 mmol) and 5-methoxy-1-methyl-1H-indole-2-carbonyl chloride (from 5-methoxy-1-methyl-1H-indole-2-carboxylic acid; 0.522 g, 2.54 mmol) in pyridine (20 mL) for 1 h. The intermediate amide was refluxed in 5% aqueous KOH (20 mL)/EtOH (10 mL) for 0.5 h to give the product (0.588 g, 80%) as a solid. ¹H NMR (DMSO-d₆) δ ppm 12.41 (bs, 1H), 8.15 (dd, 1H, J=7.9, 1.2 Hz), 7.84 (ddd, 1H, J=8.2, 7.2, 1.2 Hz), 7.74 (dd, 1H, J=8.1, 0.5 Hz), 7.47-7.53 (m, 2H), 7.39 (s, 1H), 7.14 (d, 1H, J=2.4 Hz), 6.97 (dd, 1H, J=9.0, 2.4 Hz), 4.16 (s, 3H), 3.80 (s, 3H). ACPI-MS Found: [M+H]⁺=306.

### Example 2.29

2-(7-Methyl-1-benzofuran-2-yl)-4(3H)-quinazolinone (C: R═H, R′=7-methyl-1-benzofuran-2-yl). The intermediate amide (E: R═H, R′=7-methyl-1-benzofuran-2-yl) was synthesised by refluxing 2-aminobenzamide (0.103 g, 0.757 mmol) and 7-methyl-1-benzofuran-2-carbonyl chloride (from 7-methyl-1-benzofuran-2-carboxylic acid, 0.140 g, 0.795 mmol) in pyridine (10 mL) for 1 h. The intermediate amide was refluxed in 5% aqueous KOH (10 mL)/EtOH (5 mL) for 1 h to give the product (0.125 g, 60%) as a solid. ¹H NMR (DMSO-d₆) δ ppm 12.75 (bs, 1H), 8.17 (dd, 1H, J=7.8, 1.2 Hz), 7.98 (s, 1H), 7.86 (ddd, 1H, J=8.3, 7.3, 1.6 Hz), 7.79 (dd, 1H, J=8.2, 0.7 Hz), 7.62 (dd, 1H, J=7.5, 0.7 Hz), 7.55 (ddd, 1H, J=8.1, 7.1, 1.2 Hz), 7.23-7.32 (m, 2H), 2.60 (s, 3H). ACPI-MS Found: [M+H]⁺=277.

### Example 2.30

2-(7-Methoxy-1-benzofuran-2-yl)-4(3H)-quinazolinone (C: R═H, R′=7-methoxy-1-benzofuran-2-yl). The intermediate amide (E: R═H, R′=7-methoxy-1-benzofuran-2-yl) was synthesised by refluxing 2-aminobenzamide (0.270 g, 1.98 mmol) and 7-methoxy-1-benzofuran-2-carbonyl chloride (from 7-methoxy-1-benzofuran-2-carboxylic acid; 0.400 g, 2.08 mmol) in pyridine (10 mL) for 1 h. The intermediate amide was refluxed in 5% aqueous KOH (20 mL)/EtOH (10 mL) for 1 h to give the product (0.450 g, 78%) as a solid. ¹H NMR (DMSO-d₆) δ ppm 12.0 (b, 1H), 8.14 (dd, 1H, J=8.0, 1.2 Hz), 7.98 (s, 1H), 7.74-7.84 (m, 2H), 7.50 (ddd, 1H, J=8.1, 6.9, 1.4 Hz), 7.34 (dd, 1H, J=7.9, 0.9 Hz), 7.26 (t, 1H, J=7.9 Hz), 7.08 (dd, 1H, J=7.8, 0.7 Hz), 4.00 (s, 3H). ACPI-MS Found: [M+H]⁺=293.

### Example 2.31

2-(1H-Indol-2-yl)-4(3H)-quinazolinone (C: R═H, R′=1H-indol-2-yl). The intermediate amide (E: R═H, R′=1H-indol-2-yl) was synthesised by refluxing 2-aminobenzamide (1.52 g, 11.2 mmol) and 1H-indole-2-carbonyl chloride (from 1H-indole-2-carboxylic acid; 1.996 g, 12.4 mmol) in pyridine (60 mL) for 2 h. The intermediate amide was refluxed in 5% aqueous KOH (100 mL)/EtOH (50 mL) for 1 h to give the product (2.34 g, 80%) as a solid. ¹H NMR (DMSO-d₆) δ ppm 12.58 (s, 1H), 11.76 (s, 1H), 8.16 (dd, 1H, J=7.9, 1.2 Hz), 7.85 (ddd, 1H, J=8.1, 7.2, 1.6 Hz), 7.74 (d, 1H, J=7.6 Hz), 7.60-7.68 (m, 2H), 7.47-7.57 (m, 2H), 7.23 (ddd, 1H, J=8.2, 7.0, 1.1 Hz), 7.06 (td, 1H, J=7.5, 0.9 Hz). ACPI-MS Found: [M+H]⁺=262.

### Example 2.32

2-(1-Methyl-1H-indol-2-yl)-4(3H)-quinazolinone (C: R═H, R′=1-methyl-1H-indol-2-yl). The intermediate amide (E: R═H, R′=1-methyl-1H-indol-2-yl) was synthesised by refluxing 2-aminobenzamide (0.370 g, 2.72 mmol) and 1H-indole-2-carbonyl chloride (from 1H-indole-2-carboxylic acid; 0.500 g, 2.85 mmol) in pyridine (15 mL) for 15 min. The intermediate amide was refluxed in 5% aqueous KOH (20 mL)/EtOH (10 mL) for 5 min to give the product (0.470 g, 63%) as a solid. ¹H NMR (DMSO-d₆) δ ppm 12.44 (bs, 1H), 8.16 (dd, 1H, J=7.9, 1.3 Hz), 7.84 (ddd, 1H, J=7.5, 7.2, 1.5 Hz), 7.75 (d, 1H, J=7.7 Hz), 7.67 (d, 1H, J=7.9 Hz), 7.59 (d, 1H, J=8.4 Hz), 7.53 (td, 1H, J=7.5, 1.0 Hz), 7.47 (s, 1H), 7.33 (ddd, 1H, J=7.8, 7.0, 1.0 Hz), 7.14 (td, 1H, J=7.4, 0.7 Hz), 4.20 (s, 3H). ACPI-MS Found: [M+H]⁺=276.

### 3. Ester Route (Scheme 3):

A solution of an anthranilate ester (F) and an acid chloride (R′COCl) was refluxed in pyridine or other suitable solvent with a catalytic amount of 4-N,N-dimethylaminopyridine or other suitable catalyst, followed by quenching the reaction with ice and isolation of the intermediate ester (G). This was then heated under reflux in methanolic ammonia for a specified time, and solvent was removed until the entire quinazolinone product (C) had precipitated from solution.

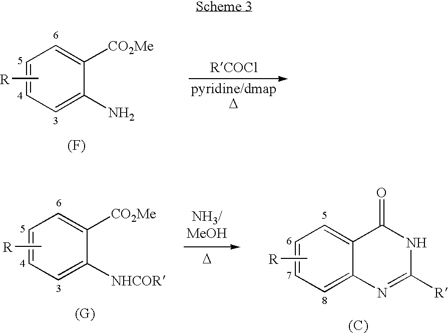

### Example 3.1

2-(1-Benzofuran-2-yl)[3,2-d]pyrimidin-4(3H)-one (C: R=5-aza, R′=benzofuran-2-yl). The intermediate ester (G: R=6-aza, R′=benzofuran-2-yl) was synthesised by refluxing methyl 3-amino-2-pyridinecarboxylate (0.250 g, 1.64 mmol) and 1-benzofuran-2-carbonyl chloride (from benzo[b]furan-2-carboxylic acid, 0.300 g, 1.85 mmol) in pyridine (10 mL) for 1 h, to give the ester (0.329 g, 68%). The intermediate ester (0.179 g, 0.604 mmol) was refluxed in methanolic ammonia (7 M, 15 mL) for 23 h to give 2-(1-benzofuran-2-yl)pyrido[3,2-d]pyrimidin-4(3H)-one (0.126 g, 79%). ¹H NMR (DMSO-d₆) δ ppm 13.02 (bs, 1H), 8.79 (dd, 1H, J=4.3, 1.4 Hz), 8.19 (dd, 1H, J=8.3, 1.4 Hz), 8.10 (d, 1H, J=0.3 Hz), 7.80-7.86 (m, 2H), 7.75 (dd, 1H, J=8.4, 0.4 Hz), 7.51 (ddd, 1H, J=8.3, 7.3, 1.2 Hz), 7.37 (td, 1H, J=7.5, 0.6 Hz). ACPI-MS Found: [M+H]⁺=264.

### Example 3.2

2-(1-Benzofuran-2-yl)-5-methyl-4(3H)-quinazolinone (C: R=5-Me, R′=benzofuran-2-yl). The intermediate ester (G: R=6-Me, R′=benzofuran-2-yl) was synthesised by refluxing methyl 2-amino-6-methylbenzoate (0.327 g, 1.98 mmol) [Z.-L. Zhou et al., Bioorganic Med. Chem., 2003, 11, 1769] and 1-benzofuran-2-carbonyl chloride (0.400 g, 2.21 mmol) in pyridine (10 mL) for 1.5 h. The intermediate ester was refluxed in concentrated methanolic ammonia (25 mL) for 110 h to give the product (0.350 g, 61%) as a solid. ¹H NMR (DMSO-d₆) δ ppm 12.47 (bs, 1H), 8.03 (s, 1H), 7.81 (d, 1H, J=7.6 Hz), 7.75 (dd, 1H, J=8.6, 0.6 Hz), 7.67 (t, 1H, J=8.0 Hz), 7.59 (bd, 1H, J=7.7 Hz), 7.49 (td, 1H, J=8.4, 1.2 Hz), 7.36 (td, 1H, J=7.5, 0.8 Hz), 7.28 (bd, 1H, J=7.2 Hz), 2.82 (s, 3H). ACPI-MS Found: [M+H]⁺=277.

### Example 3.3

2-(1-Benzofuran-2-yl)-5-nitro-4(3H)-quinazolinone (C: R=5-NO₂, R′=benzofuran-2-yl). The intermediate ester (G: R=6-NO₂, R′=benzofuran-2-yl) was synthesised by refluxing methyl 2-amino-6-nitrobenzoate (0.511 g, 2.61 mmol) [W. S. Saari et al., J. Het. Chem., 1986, 23, 1253] and 1-benzofuran-2-carbonyl chloride (0.520 g, 2.88 mmol) in pyridine (10 mL) for 1 h. The ester in concentrated methanolic ammonia (25 mL) was refluxed for 40 h to give the product (0.452 g, 56%) as a solid. ¹H NMR (DMSO-d₆) δ ppm 14.60 (bs, 1H), 8.68 (dd, 1H, J=8.3, 1.0 Hz), 7.83 (d, 1H, J=7.6 Hz), 7.70 (dd, 1H, J=8.3, 0.5 Hz), 7.65 (d, 1H, J=0.7 Hz), 7.52 (td, 1H, J=7.8, 1.2 Hz), 7.44 (t, 1H, J=8.1 Hz), 7.37 (td, 1H, J=7.5, 0.7 Hz), 7.21 (dd, 1H, J=7.8, 1.0 Hz). ACPI-MS Found: [M+H]⁺=308.

### Example 3.4

2-(1-Benzofuran-2-yl)-5-methoxy-4(3H)-quinazolinone (C: R=5-OMe, R′=1-benzofuran-2-yl). The intermediate ester (G: R=6-OMe, R′=benzofuran-2-yl) was synthesised by refluxing methyl 2-amino-6-methoxybenzoate (0.340 g, 1.88 mmol) (M. Jubault et al, Bull Chem. Soc. Fr., (11972) 2355) and 1-benzofuran-2-carbonyl chloride (0.280 g, 2.10 mmol) in pyridine (5 mL) for 1 h to give the intermediate ester. The intermediate ester was refluxed in methanolic ammonia (7 M, 15 mL) for 39 h, this was not sufficient to effect cyclisation. The crude material was cyclised by refluxing with 5% KOH (30 mL)/EtOH (15 mL) for 1 h to give the product (0.415 g, 76%) as a solid. ¹H NMR (DMSO-d₆) δ ppm 12.71 (bs, 1H), 7.99 (d, 1H, J=0.7 Hz), 7.80 (d, 1H, J=7.5 Hz), 7.71-7.76 (m, 2H), 7.57 (d, 1H, J=3.0 Hz), 7.44-7.51 (m, 2H), 7.35 (td, 1H, J=7.6, 0.9 Hz), 4.08 (s, 3H). ACPI-MS Found: [M+H]⁺=293.

### Example 3.5

2-(1-Benzofuran-2-yl)pyrido[4,3-d]pyrimidin-4(3H)-one (C: R=6-aza, R′=benzofuran-2-yl). The intermediate ester (G: R=5-aza, R′=benzofuran-2-yl) was synthesised by refluxing methyl 4-aminonicotinate (0.325 g, 2.14 mmol) and 1-benzofuran-2-carbonyl chloride (0.470 g, 2.60 mmol) in pyridine (15 mL) for 2 h. The ester in concentrated methanolic ammonia (20 mL) was refluxed for 24 h to give the product (0.308 g, 55%) as a solid. ¹H NMR (DMSO-d₆) δ ppm 13.0 (bs, 1H), 9.27 (s, 1H), 8.80 (d, 1H, J=5.6 Hz), 8.09 (s, 1H), 7.84 (d, 1H, J=7.6 Hz), 7.76 (dd, 1H, J=8.4, 0.6 Hz), 7.63 (d, 1H, J=5.6 Hz), 7.52 (ddd, 1H, J=8.3, 7.3, 1.2 Hz), 7.38 (td, 1H, J=7.5, 0.8 Hz). ACPI-MS Found: [M+H]⁺=264.

### Example 3.6

2-(1-Benzofuran-2-yl)-6-methoxy-4(3H)-quinazolinone (C: R=6-OMe, R′=benzofuran-2-yl). The intermediate ester (G: R=6-OMe, R′=benzofuran-2-yl) was synthesised by refluxing methyl 2-amino-5-methoxybenzoate (0.437 g, 2.41 mmol) (C. Theeraladanon et al., 60(13), (2004), 3017) and 1-benzofuran-2-carbonyl chloride (0.480 g, 2.66 mmol) in pyridine (10 mL) for 0.5 h. The ester was refluxed in concentrated methanolic ammonia-(20 mL) for 48 h to give the product (0.415 g, 59%) as a solid. ¹H NMR (DMSO-d₆) δ ppm 12.70 (bs, 1H), 8.00 (d, 1H, J=0.7 Hz), 7.80 (bd, 1H, J=7.6 Hz), 7.70-7.77 (m, 2H), 7.57 (d, 1H, J=3.0 Hz), 7.43-7.50 (m, 2H), 7.35 (td, 1H, J=7.5, 0.7 Hz), 3.90 (s, 3H). ACPI-MS Found: [M+H]⁺=293.

### Example 3.7

2-(1-Benzofuran-2-yl)-7-(trifluoromethyl)-4(3H)-quinazolinone (C: R=7-CF₃, R′=benzofuran-2-yl). The intermediate ester (G: R=4-CF₃, R′=benzofuran-2-yl) was synthesised by refluxing methyl 2-amino-4-(trifluoromethyl)benzoate (0.464 g, 2.12 mmol) (D. T. Hill et al., J. Med. Chem., 26(6), (1983), 865) and 1-benzofuran-2-carbonyl chloride (0.420 g, 2.32 mmol) in pyridine (20 mL) for 1 h. The ester was refluxed in methanolic ammonia (15 mL, 1.25 M) for 48 h to give 2-(1-benzofuran-2-yl)-7-(trifluoromethyl)-4(3H)-quinazolinone (0.562 g, 80%). ¹H NMR (DMSO-d₆) δ ppm 13.0 (bs, 1H), 8.33 (d, 1H, J=8.3 Hz), 8.04-8.09 (m, 2H), 7.84 (d, 1H, J=7.6 Hz), 7.80 (dd, 1H, J=8.3, 1.4 Hz), 7.75 (dd, 1H, J=8.3, 0.8 Hz), 7.46-7.52 (m, 1H), 7.37 (td, 1H, J=7.6, 0.8 Hz). ACPI-MS Found: [M+H]⁺=331.

### Example 3.8

2-(1-Benzofuran-2-yl)-7-methoxy-4(3H)-quinazolinone (C: R=7-OMe, R′=benzofuran-2-yl). The intermediate ester (G: R=7-OMe, R′=benzofuran-2-yl) was synthesised by refluxing methyl 2-amino-4-methoxybenzoate (0.522 g, 2.91 mmol) and 1-benzofuran-2-carbonyl chloride (0.560 g, 3.10 mmol) in pyridine (10 mL) for 0.5 h, to give the ester (0.916 g, 97%). The ester (0.448 g, 1.38 mmol) was refluxed in methanolic ammonia (7 M, 25 mL) for 64 h to give the product (0.280 g, 69%) as a solid. ¹H NMR (DMSO-d₆) δ ppm 12.0 (bs, 1H), 8.01-8.07 (m, 2H), 7.82 (d, 1H, J=7.7 Hz), 7.73 (dd, 1H, J=8.3, 0.5 Hz), 7.49 (td, 1H, J=7.7, 1.2 Hz), 7.30 (td, 1H, J=7.5, 0.7 Hz), 7.25 (d, 1H, J=2.4 Hz), 7.11 (dd, 1H, J=8.8, 2.5 Hz), 3.93 (s, 3H). ACPI-MS Found: [M+H]⁺=293.

### Example 3.9

2-(1-Benzofuran-2-yl)-4-oxo-3,4-dihydro-7-quinazolinecarboxamide (C: R=7-CONH₂, R′=1-benzofuran-2-yl). The intermediate ester (G: R=4-CONH₂, R′=1′-benzofuran-2-yl) was synthesised by refluxing methyl 2-amino-4-(aminocarbonyl)benzoate (0.342 g, 1.76 mmol) and 1-benzofuran-2-carbonyl chloride (0.350 g, 1.94 mmol) in pyridine (10 mL) for 1 h. The intermediate ester was refluxed in concentrated methanolic ammonia (25 mL) for 18 h to give the product (0.230 g, 43%) as a solid. ¹H NMR (DMSO-d₆) δ ppm 12.85 (bs, 1H), 8.30 (d, 1H, J=1.5 Hz), 8.24 (bs, 1H), 8.20 (d, 1H, J=8.2 Hz), 8.06 (s, 1H), 7.95 (dd, 1H, J=8.2, 1.6 Hz), 7.84 (d, 1H, J=7.6 Hz), 7.75 (dd, 1H, J=8.3, 0.6 Hz), 7.62 (bs, 1H), 7.51 (ddd, 1H, J=8.4, 7.2, 1.3 Hz), 7.37 (td, 1H, J=7.5, 0.9 Hz). ACPI-MS Found: [M+H]⁺=306.

### Example 3.10

2-(1-Benzofuran-2-yl)pyrido[2,3-d]pyrimidin-4(3H)-one (C: R=8-aza, R′=benzofuran-2-yl). The intermediate ester (G: R=3-aza, R′=benzofuran-2-yl) was synthesised by refluxing methyl 2-aminonicotinate (0.250 g, 1.64 mmol) and 1-benzofuran-2-carbonyl chloride (from benzo[b]furan-2-carboxylic acid, 0.300 g, 1.85 mmol) in pyridine (10 mL) for 0.5 h, to give the ester (0.329 g, 68%). The ester (0.130 g, 0.439 mmol) was refluxed in methanolic ammonia (20 mL) for 48 h to give the product (0.099 g, 86%) as a solid. ¹H NMR (DMSO-d₆) δ ppm 13.04 (bs, 1H), 8.99 (d, 1H, J=2.6 Hz), 8.52 (dd, 1H, J=7.7, 1.3 Hz), 8.12 (s, 1H), 7.85 (d, 1H, J=7.8 Hz), 7.76 (d, 1H, J=8.3 Hz), 7.48-7.59 (m, 2H), 7.39 (t, 1H, J=7.5 Hz). ACPI-MS Found: [M+H]⁺=264.

### Example 3.11

2-(1-Benzofuran-2-yl)-8-phenyl-4(3H)-quinazolinone (C: R=8-Ph, R′=benzofuran-2-yl). The intermediate ester (G: R=3-Ph, R′=benzofuran-2-yl) was synthesised by refluxing methyl 2-amino-3-phenylbenzoate (0.311 g, 1.37 mmol) (L. Bin et al., Tet. Lett., 46(11), (2005), 1779) and 1-benzofuran-2-carbonyl chloride (0.260 g, 1.44 mmol) in pyridine (20 mL) for 1 h. The ester was refluxed in concentrated methanolic ammonia (25 mL) for 48 h to give the product (0.239 g, 52%) as a solid. ¹H NMR (DMSO-d₆) δ ppm 12.80 (bs, 1H), 8.20 (dd, 1H, J=7.9, 1.6 Hz), 7.89 (dd, 1H, J=7.4, 1.6 Hz), 7.85 (d, 1H, J=0.9 Hz), 7.81 (d, 1H, J=7.4 Hz), 7.73-7.77 (m, 2H), 7.58-7.67 (m, 2H), 7.50-7.56 (m, 2H), 7.42-7.49 (m, 2H), 7.35 (td, 1H, J=7.5, 0.9 Hz). ACPI-MS Found: [M+H]⁺=339.

### Example 3.12

2-(1-Benzofuran-2-yl)-8-(trifluoromethyl)-4(3H)-quinazolinone (C: R=8-CF₃, R′=benzofuran-2-yl). The intermediate ester (G: R=3-CF₃, R′=benzofuran-2-yl) was synthesised by refluxing methyl 2-amino-3-(trifluoromethyl)benzoate (0.310 g, 1.41 mmol) (Y. Shpernat et al., PCT Int. Appl. (2005), WO 2005007634) and 1-benzofuran-2-carbonyl chloride (from benzo[b]furan-2-carboxylic acid, 0.250 g, 1.54 mmol) in pyridine (10 mL) for 0.5 h. The ester was refluxed in concentrated methanolic ammonia (25 mL) for 48 h to give the product as a solid, which was used in the subsequent step without purification. ACPI-MS Found: [M+H]⁺=331.

### Example 3.13

2-(1-Benzofuran-2-yl)-8-nitro-4(3H)-quinazolinone (C: R=8-NO₂, R′=benzofuran-2-yl). The intermediate ester (G: R=3-NO₂, R′=benzofuran-2-yl) was synthesised by refluxing methyl 2-amino-3-nitrobenzoate (0.494 g, 2.35 mmol) and 1-benzofuran-2-carbonyl chloride (from benzo[b]furan-2-carboxylic acid, 0.400 g, 2.47 mmol) in pyridine (10 mL) for 1 h. The ester was refluxed in concentrated methanolic ammonia (25 mL) for 64 h to give the product (0.429 g, 59%) as a solid. ¹H NMR (DMSO-d₆) δ ppm 8.25 (dd, 1H, J=7.9, 1.5 Hz), 8.06 (dd, 1H, J=7.7, 1.5 Hz), 7.77 (bdd, 1H, J=7.5, 0.4 Hz), 7.69-7.72 (m, 2H), 7.37-7.45 (m, 2H), 7.31 (td, 1H, J=7.6, 0.8 Hz), 7.0-8.2 (bs, 1H). ACPI-MS Found: [M+H]⁺=308.

### Example 3.14

2-(1-Benzofuran-2-yl)benzo[g]quinazolin-4(3H)-one (C: R=6,7-benz, R′=benzofuran-2-yl). The intermediate ester (G: R=4,5-benz, R′=benzofuran-2-yl) was synthesised by refluxing methyl 3-amino-2-naphthoate (0.545 g, 2.71 mmol) (C. Theeraladanon et al., 60(13), (2004), 3017) and 1-benzofuran-2-carbonyl chloride (0.540 g, 2.99 mmol) in pyridine (20 mL) for 2 h. The ester was refluxed in methanolic ammonia (10 mL, 7 M) for 64 h to give the product (0.617 g, 73%) as a solid. ¹H NMR (DMSO-d₆) δ ppm 12.55 (bs, 1H), 8.87 (s, 1H), 8.36 (s, 1H), 8.22 (d, 1H, J=8.3 Hz), 8.13 (d, 1H, J=8.3 Hz), 8.09 (s, 1H), 7.84 (d, 1H, J=7.7 Hz), 7.78 (d, 1H, J=8.3 Hz), 7.69 (t, 1H, J=7.3 Hz), 7.61 (t, 1H, J=7.3 Hz), 7.51 (td, 1H, J=7.8, 1.1 Hz), 7.38 (td, 1H, J=7.5, 0.6 Hz). ACPI-MS Found: [M+H]⁺=313.

### Example 3.15

2-(1-Benzofuran-2-yl)-6,8-dichloro-4(3H)-quinazolinone (C: R=6,8-diCl, R′=benzofuran-2-yl). The intermediate ester (G: R=3,5-diCl, R′=benzofuran-2-yl) was synthesised by refluxing methyl 3,5-dichloro-2-aminobenzoate (1.02 g, 4.63 mmol) and 1-benzofuran-2-carbonyl chloride (from benzo[b]furan-2-carboxylic acid, 0.839 g, 5.17 mmol) in pyridine (40 mL) for 2 h. The ester was refluxed in methanolic ammonia (10 mL, 7 M) for 60 h to give the product (1.10 g, 72%) as a solid. ¹H NMR (DMSO-d₆) δ ppm 13.09 (bs, 1H), 8.13 (d, 1H, J=2.4 Hz), 8.08 (d, 1H, J=0.9 Hz), 8.04 (d, 1H, J=2.4 Hz), 7.84 (bd, 1H, J=7.5 Hz), 7.76 (dd, 1H, J=8.4, 0.8 Hz), 7.51 (ddd, 1H, J=8.4, 7.5, 1.3 Hz), 7.37 (td, 1H, J=7.5, 0.8 Hz). ACPI-MS Found: [M+H]⁺=331, 333, 335.

### Example 3.16

2-(1-Benzofuran-2-yl)-6,8-dibromo-4(3H)-quinazolinone (C: R=6,8-diBr, R′=benzofuran-2-yl). The intermediate ester (G: R=3,5-diBr, R′=benzofuran-2-yl) was synthesised by refluxing methyl 3,5-dibromo-2-aminobenzoate (0.650 g, 2.10 mmol) and 1-benzofuran-2-carbonyl chloride (from benzo[b]furan-2-carboxylic acid, 0.360 g, 2.22 mmol) in pyridine (10 mL) for 1 h, to give the ester (0.729 g, 76%). The ester (0.206 g, 0.455 mmol) was refluxed in concentrated methanolic ammonia (25 mL) for 64 h to give the product (0.191 g, 100%) as a solid. ¹H NMR (DMSO-d₆) δ ppm 13.0 (bs, 1H), 8.30 (d, 1H, J=2.0 Hz), 8.20 (d, 1H, J=2.0 Hz), 8.10 (s, 1H), 7.84 (d, 1H, J=7.5 Hz), 7.74 (d, 1H, J=8.3 Hz), 7.50 (td, 1H, J=7.8 Hz), 7.37 (t, 1H, J=7.5 Hz). ACPI-MS Found: [M+H]⁺=418, 420, 422.

### Example 3.17

2-(1-Benzofuran-2-yl)-7,8-dimethyl-4(3H)-quinazolinone (C: R=7,8-diMe, R′=benzofuran-2-yl). The intermediate ester (G: R=3,4-diMe, R′=benzofuran-2-yl) was synthesised by refluxing methyl 3,4-dimethyl-2-aminobenzoate (0.617 g, 3.44 mmol) (G. E. Hardtmann et al., (1973), U.S. Pat. No. 3,763,163) and 1-benzofuran-2-carbonyl chloride (from benzo[b]furan-2-carboxylic acid, 0.710 g, 3.93 mmol) in pyridine (30 mL) for 2 h, to give the ester (1.024 g, 92%). The ester (0.927 g, 2.87 mmol) was refluxed in concentrated methanolic ammonia (15 mL, 7 M) for 23 h to give 2-(1-benzofuran-2-yl)-7,8-dimethyl-4(3H)-quinazolinone (0.343 g, 41%). ¹H NMR (DMSO-d₆) δ ppm 12.52 (bs, 1H), 8.01 (s, 1H), 7.91 (d, 1H, J=0.7 Hz), 7.82 (d, 1H, J=7.5 Hz), 7.75 (dd, 1H, J=8.4, 0.6 Hz), 7.48 (td, 1H, J=7.5, 1.3 Hz), 7.33-7.39 (m, 2H), 2.60 (s, 3H), 2.43 (s, 3H). ACPI-MS Found: [M+H]⁺=291.

### Preparation of the 4-aminoquinazoline compounds of the invention

### Synthesis of 4-aminoquinazolines

### Scheme 4

Conversion of the quinazolinones (C) to the chloroquinazolines (H) can be performed by refluxing the substrate in thionyl chloride, followed by removal of excess thionyl chloride under reduced pressure. Alternatively, the quinazolinones (C) can be refluxed with excess POCl₃ and Me₄N⁺Cl⁻ (2 equiv.), followed by removal of excess POCl₃ under reduced pressure. The crude chloroquinazolines (H) can be isolated by partitioning the resulting residues between dichloromethane and sat. aq. K₂CO₃, and purified by filtration through a plug of alumina using dichloromethane as the eluent. The chloroquinazolines (H) are then treated with the amines H₂NR¹ (3 equiv.) under reflux in dioxane or another suitable solvent for a specified time. Removal of the solvent gives the crude aminoquinazolines (I), which are partitioned between aqueous K₂CO₃/EtOAc, washed with water and dried to give the pure products. In certain instances the 4-aminoquinazolines (I) were converted to their HCl salts by stirring with methanolic HCl (10 equiv.), removal of excess HCl followed by recrystallisation from EtOAc/MeOH.

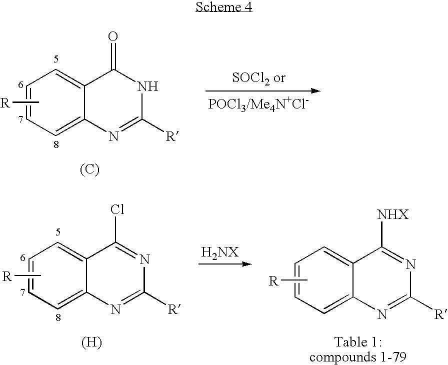

### Example 4.1

N¹-[2-(1-benzofuran-2-yl)-4-quinazolinyl]-N²,N²-dimethyl-1,2-ethanediamine (1). A mixture of 2-(1-benzofuran-2-yl)-4(3H)-quinazolinone (C: R═H, R′=benzofuran-2-yl) (0.917 g, 3.50 mmol) and tetramethylammonium chloride (0.794 g, 7.24 mmol) in POCl₃ (24 mL) was refluxed for 15 min to give 2-(1-benzofuran-2-yl)-4-chloroquinazoline (H: R═H, R′=benzofuran-2-yl) (0.874 g, 93%). A solution of the chloroquinazoline (0.111 g, 0.395 mmol) and N¹,N¹-dimethyl-1,2-ethanediamine (0.13 mL, 1.18 mmol) in dioxane (15 mL) was refluxed for 2 h, workup gave 1 (0.109 g, 83%) as a solid. ¹H NMR (DMSO-d₆) δ ppm 8.31 (t, 1H, J=5.5 Hz), 8.24 (d, 1H, J=8.3 Hz), 7.75-7.82 (m, 3H), 7.71 (dd, 1H, J=8.3, 0.7 Hz), 7.68 (d, 1H, J=0.9 Hz), 7.49-7.56 (m, 1H), 7.41 (ddd, 1H, J=8.4, 7.5, 1.3 Hz), 7.31 (td, 1H, J=7.5, 0.9 Hz), 3.78 (dt, 1H, J=6.8, 5.5 Hz), 2.61 (t, 2H, J=6.8 Hz), 2.26 (s, 6H). ACPI-MS Found: [M+H]⁺=333.

### Example 4.2

N¹-[2-(1-benzofuran-2-yl)-4-quinazolinyl]-N¹,N²,N²-trimethyl-1,2-ethanediamine dihydrochloride (2). A solution of 2-(1-benzofuran-2-yl)-4-chloroquinazoline (H: R═H, R′=benzofuran-2-yl) (0.027 g, 0.096 mmol) and N¹,N¹,N²-trimethyl-1,2-ethanediamine (0.04 mL, 0.3 mmol) in dioxane (5 mL) was refluxed for 1 h, workup and conversion to the hydrochloride salt gave 3 (38 mg, 95%) as a solid. ¹H NMR (DMSO-d₆) δ ppm 10.7 (bs, 1H), 8.39 (d, 1H, J=8.4 Hz), 8.23 (bs, 1H), 8.04 (d, 1H, J=8.2 Hz), 7.95 (t, 1H, J=7.5 Hz), 7.84 (d, 1H, J=7.6 Hz), 7.78 (dd, 1H, J=8.4, 0.5 Hz), 7.63 (t, 1H, J=7.5 Hz), 7.53 (td, 1H, J=7.8, 1.0 Hz), 7.40 (t, 1H, J=7.3 Hz), 4.39 (t, 2H, J=6.4 Hz), 3.67 (s, 3H), 3.48-3.57 (m, 2H), 2.92 (d, 6H, J=4.9 Hz). ACPI-MS Found: [M+H]⁺=347.

### Example 4.3

N¹-[2-(1-benzofuran-2-yl)-4-quinazolinyl]-N³,N³-dimethyl-1,3-propanediamine dihydrochloride (3). A solution of 2-(1-benzofuran-2-yl)-4-chloroquinazoline (H: R═H, R′=benzofuran-2-yl) (0.822 g, 2.93 mmol) and N¹,N¹-dimethyl-1,3-propanediamine (1.0 mL, 8.6 mmol) in dioxane (40 mL) was refluxed for 2 h, workup and conversion to the hydrochloride salt gave 3 (1.064 g, 87%) as a solid. ¹H NMR (DMSO-d₆) δ ppm 14.7 (b, 1H), 10.42 (bs, 1H), 10.07 (b, 1H), 8.67 (d, 1H, J=8.2 Hz), 8.39 (s, 1H), 8.13 (d, 1H, J=8.3 Hz), 8.01 (td, 1H, J=7.7, 0.7 Hz), 7.90 (d, 1H, J=7.7 Hz), 7.82 (dd, 1H, J=8.4, 0.7 Hz), 7.73 (td, 1H, J=7.3, 0.8 Hz), 7.59 (td, 1H, J=7.8, 1.1 Hz), 7.44 (td, 1H, J=7.5, 0.7 Hz), 3.85-3.93 (m, 2H), 3.20-3.28 (m, 2H), 2.76 (d, 6H, J=5.0 Hz), 2.17-2.26 (m, 2H). ACPI-MS Found: [M+H]⁺=347.

### Example 4.4

N¹-[2-(1-benzofuran-2-yl)-4-quinazolinyl]-N⁴,N⁴-dimethyl-1,4-butanediamine dihydrochloride (4). A solution of 2-(1-benzofuran-2-yl)-4-chloroquinazoline (H: R═H, R′=benzofuran-2-yl) (0.274 g, 0.976 mmol) and N¹,N¹-dimethyl-1,4-butanediamine (0.35 mL, 3.0 mmol) in dioxane (30 mL) was refluxed for 1.5 h, workup and conversion to the hydrochloride salt gave 4 (0.169 g, 40%) as a solid. ¹H NMR (DMSO-d₆) δ ppm 10.82 (bs, 1H), 8.38 (d, 1H, J=8.4 Hz), 8.32 (bs, 1H), 8.17 (d, 1H, J=8.2 Hz), 8.00 (td, 1H, J=8.2, 0.9 Hz), 7.93 (d, 1H, J=7.6 Hz), 7.82 (dd, 1H, J=8.4, 0.6 Hz), 7.67 (td, 1H, J=7.8, 1.0 Hz), 7.57 (ddd, 1H, J=8.3, 7.3, 1.2 Hz), 7.43 (td, 1H, J=7.5, 0.7 Hz), 4.02-4.10 (m, 2H), 3.08-3.16 (m, 2H), 2.71 (d, 6H, J=5 Hz), 1.80-1.93 (m, 4H). ACPI-MS Found: [M+H]⁺=361.

### Example 4.5

N¹-[2-(1-benzofuran-2-yl)-4-quinazolinyl]-N³,N³-diethyl-1,3-propanediamine dihydrochloride (5). A solution of 2-(1-benzofuran-2-yl)-4-chloroquinazoline (H: R═H, R′=benzofuran-2-yl) (0.180 g, 0.641 mmol) and N¹,N¹-diethyl-1,3-propanediamine (0.3 mL, 1.9 mmol) in dioxane (10 mL) was refluxed for 2 h, workup and conversion to the hydrochloride salt gave 5 (0.252 g, 88%) as a solid. ¹H NMR (DMSO-d₆) δ ppm 10.54 (bs, 1H), 10.43 (bs, 1H), 8.68 (d, 1H, J=8.2 Hz), 8.39 (s, 1H), 8.13 (d, 1H, J=8.3 Hz), 8.01 (t, 1H, J=7.3 Hz), 7.90 (d, 1H, J=7.7 Hz), 7.82 (dd, 1H, J=8.4, 0.5 Hz), 7.73 (t, 1H, J=7.4 Hz), 7.59 (td, 1H, J=7.8, 1.1 Hz), 7.44 (td, 1H, J=7.3, 0.5 Hz), 3.87-3.75 (m, 2H), 3.20-3.26 (m, 2H), 3.06-3.14 (m, 4H), 2.16-2.25 (m, 2H), 1.22 (t, 6H, J=7.2 Hz). ACPI-MS Found: [M+H]⁺=375.

### Example 4.6

N¹-[2-(1-benzofuran-2-yl)-4-quinazolinyl]-N³,N³-dipropyl-1,3-propanediamine (6). A solution of 2-(1-benzofuran-2-yl)-4-chloroquinazoline (H: R═H, R′=benzofuran-2-yl) (0.270 g, 0.962 mmol) and N¹,N¹-dipropyl-1,3-propanediamine (0.45 mL, 2.84 mmol) in dioxane (30 mL) was refluxed for 2 h, workup gave 6 (0.357 g, 92%) as a solid. ¹H NMR (DMSO-d₆) δ ppm 8.40 (t, 1H, J=5.3 Hz), 8.23 (d, 1H, J=8.3 Hz), 7.77-7.81 (m, 2H), 7.75 (d, 1H, J=7.4 Hz), 7.71 (dd, 1H, J=8.3, 0.7 Hz), 7.67 (d, 1H, J=0.9 Hz), 7.50-7.56 (m, 1H), 7.41 (ddd, 1H, J=8.4, 7.5, 1.3 Hz), 7.31 (td, 1H, J=7.5, 0.8 Hz), 3.68 (td, 1H, J=6.3, 5.3 Hz), 2.55 (t, 2H, J=6.9 Hz), 2.37 (t, 4H, J=7.3 Hz), 1.86 (pent, 2H, J=7.3, 6.3 Hz), 1.45 (sxt, 4H, J=7.3 Hz), 0.83 (t, 6H, J=7.3 Hz). ACPI-MS Found: [M+H]⁺=403.

### Example 4.7

N¹-[2-(1-benzofuran-2-yl)-4-quinazolinyl]-N³,N³-bis(2-hydroxyethyl)-1,3-propanediamine (7). A solution of 2-(1-benzofuran-2-yl)-4-chloroquinazoline (H: R═H, R′=benzofuran-2-yl) (0.270 g, 0.962 mmol) and N¹,N¹-bis(2-hydroxyethyl)-1,3-propanediamine (0.450 mL, 2.77 mmol) in dioxane (30 mL) was refluxed for 2 h, workup gave 7 (0.361 g, 92%) as a solid. ¹H NMR (DMSO-d₆) δ ppm 8.42 (t, 1H, J=5.2 Hz), 8.24 (d, 1H, J=8.3 Hz), 7.69-7.82 (m, 5H), 7.48-7.55 (m, 1H), 7.41 (ddd, 1H, J=8.4, 7.5, 1.3 Hz), 7.31 (td, 1H, J=7.5, 0.9 Hz), 4.37 (t, 2H, J=5.4 Hz), 3.70 (ddd, 2H, J=6.8, 6.8, 5.2 Hz), 3.48 (ddd, 4H, J=6.3, 6.3, 5.4 Hz), 2.66 (t, 2H, J=6.8 Hz), 2.58 (t, 4H, J=6.3 Hz), 1.87 (p, 2H, J=6.8 Hz). ACPI-MS Found: [M+H]⁺=407.

### Example 4.8

2-(1-Benzofuran-2-yl)-N-[3-(4-morpholinyl)propyl]-4-quinazolinamine dihydrochloride (8). A solution of 2-(1-benzofuran-2-yl)-4-chloroquinazoline (H: R═H, R′=benzofuran-2-yl) (0.376 g, 1.34 mmol) and 3-(4-morpholinyl)-propanamine (0.5 mL) in dioxane (20 mL) was refluxed for 2 h, workup and conversion to the hydrochloride salt gave 8 (0.304 g, 49%) as a solid. ¹H NMR (DMSO-d₆) δ ppm 14.6 (b, 1H), 11.44 (bs, 1H), 10.55 (bs, 1H), 8.70 (d, 1H, J=8.2 Hz), 8.44 (s, 1H), 8.16 (d, 1H, J=8.3 Hz), 8.02 (td, 1H, J=7.7, 0.8 Hz), 7.91 (d, 1H, J=7.7 Hz), 7.83 (dd, 1H, J=8.3, 0.5 Hz), 7.74 (td, 1H, J=8.0, 0.8 Hz), 7.60 (td, 1H, J=7.8, 1.2 Hz), 7.45 (td, 1H, J=7.5, 0.6 Hz), 2.95-4.00 (m, 12H), 2.23-2.35 (m, 2H). ACPI-MS Found: [M+H]⁺=389.

### Example 4.9

2-(1-Benzofuran-2-yl)-N-[3-(4-methyl-1-piperazinyl)propyl]-4-quinazolinamine (9). A solution of 2-(1-benzofuran-2-yl)-4-chloroquinazoline (H: R═H, R′=benzofuran-2-yl) (0.354 g, 1.26 mmol) and 3-(4-methyl-1-piperazinyl)propylamine (0.6 g, 3.82 mmol) in dioxane (20 mL) was refluxed for 2 h, workup gave 9 (0.334 g, 66%) as a solid. ¹H NMR (DMSO-d₆) δ ppm 8.42 (t, 1H, J=5.4 Hz), 8.23 (d, 1H, J=8.3 Hz), 7.74-7.83 (m, 3H), 7.71 (dd, 1H, J=8.3, 0.7 Hz), 7.68 (d, 1H, J=0.9 Hz), 7.48-7.55 (m, 1H), 7.41 (ddd, 1H, J=8.3, 7.3, 1.3 Hz), 7.31 (td, 1H, J=7.5, 0.9 Hz), 3.67-3.74 (m, 2H), 2.25-2.50 (m, 10H), 2.15 (s, 3H), 1.84-1.92 (m, 2H). ACPI-MS Found: [M+H]⁺=402.

### Example 4.10

2-(1-benzofuran-2-yl)-N-[3-(1-pyrrolidinyl)propyl]-4-quinazolinamine dihydrochloride (10).

Synthesis of 3-{[2-(1-benzofuran-2-yl)-4-quinazolinyl]amino}1-propanol.

A solution of 2-(1-benzofuran-2-yl)-4-chloroquinazoline (H: R═H, R′=benzofuran-2-yl) (0.287 g, 1.02 mmol) and 3-(dimethylamino)-1-propanol (0.30 mL, 3.92 mmol) in dioxane (30 mL) was refluxed for 2 h, workup gave 3-{[2-(1-benzofuran-2-yl)-4-quinazolinyl]amino}-1-propanol (0.302 g, 92%) as a solid. ¹H NMR (DMSO-d₆) δ ppm 8.36 (bt, 1H, J=5.3 Hz), 8.26 (d, 1H, J=8.3 Hz), 7.69-7.81 (m, 5H), 7.48-7.55 (m, 1H), 7.40 (td, 1H, J=7.6, 1.3 Hz), 7.31 (td, 1H, J=7.6, 0.9 Hz), 4.57 (t, 1H, J=5.2 Hz), 3.70-3.77 (m, 2H), 3.56-3.62 (m, 2H), 1.85-1.94 (m, 2H). ACPI-MS Found: [M+H]⁺=320.

Mesyl chloride (40 μL, 0.51 mmol) was added to a solution of 3-{[2-(1-benzofuran-2-yl)-4-quinazolinyl]amino}-1-propanol (0.107 g, 0.335 mmol) and triethylamine (95 μL, 0.69 mmol) in thf (10 mL) at 0° C. The solution was stirred at 0° C. until consumption of starting material was evident by t.l.c. Pyrrolidine (280 μL, 3.35 mmol) was added and the solution was refluxed for 1 h then partitioned between EtOAc/sat. aq. NaHCO₃. Column chromatography on alumina (EtOAc) gave a product which was converted to the HCl salt to give 10 (62 mg, 42%). ¹H NMR (DMSO-d₆) δ ppm 11.00 (bs, 1H), 10.46 (bs, 1H), 8.68 (d, 1H, J=8.2 Hz), 8.43 (s, 1H), 8.14 (d, 1H, J=8.3 Hz), 8.01 (t, 1H, J=7.5 Hz), 7.90 (d, 1H, J=7.7 Hz), 7.82 (d, 1H, J=8.3 Hz), 7.73 (t, 1H, J=7.6 Hz), 7.59 (t, 1H, J=7.5 Hz), 7.44 (t, 1H, J=7.5 Hz), 3.87-3.98 (m, 2H), 3.46-3.60 (m, 2H), 3.24-3.37 (m, 2H), 2.90-3.05 (m, 2H), 2.16-2.28 (m, 2H), 1.80-2.04 (m, 4H). ACPI-MS Found: [M+H]⁺=373.

### Example 4.11

N¹-[2-(1-benzofuran-2-yl)-4-quinazolinyl]-N³-cyclopropyl-1,3-propanediamine dihydrochloride (11). Mesyl chloride (55 μL, 0.70 mmol) was added to a solution of 3-{[2-(1-benzofuran-2-yl)-4-quinazolinyl]amino}-1-propanol (0.150 g, 0.470 mmol) and triethylamine (130 μL, 0.93 mmol) in thf (10 mL) at 0° C. in a sealed tube. The solution was stirred at 0° C. until consumption of starting material was evident by t.l.c. Cyclopropylamine (330 μL, 4.74 mmol) was added and the solution was refluxed for 1 h then partitioned between EtOAc/sat. aq. NaHCO₃. Column chromatography on alumina (EtOAc) gave a product which was converted to the HCl salt to give 11 (54 mg, 27%). ¹H NMR (DMSO-d₆) δ ppm 10.10 (bs, 1H), 9.25 (bs, 2H), 8.59 (d, 1H, J=8.3 Hz), 8.30 (bs, 1H), 8.07 (d, 1H, J=8.3 Hz), 7.99 (t, 1H, J=7.4 Hz), 7.89 (d, 1H, J=7.7 Hz), 7.82 (d, 1H, J=7.9 Hz), 7.72 (t, 1H, J=7.5 Hz), 7.58 (t, 1H, J=7.5 Hz), 7.43 (t, 1H, J=7.4 Hz), 3.87-3.95 (m, 2H), 3.12-3.22 (m, 2H), 2.66-2.74 (m, 1H), 2.18 (p, 2H, J=7.4 Hz), 0.90-0.97 9 m, 2H), 0.68-0.75 (m, 2H). ACPI-MS Found: [M+H]⁺=359.

### Example 4.12

N¹-[2-(1-benzofuran-2-yl)-4-quinazolinyl]-N³-methyl-1,3-propanediamine dihydrochloride (12). Mesyl chloride (55 μL, 0.70 mmol) was added to a solution of 3-{[2-(1-benzofuran-2-yl)-4-quinazolinyl]amino}-1-propanol (0.150 g, 0.470 mmol) and triethylamine (130 μL, 0.93 mmol) in thf (10 mL) at 0° C. in a sealed tube. The solution was stirred at 0° C. until consumption of starting material was evident by t.l.c. Methylamine (approximately 0.5 mL) was added and the solution was refluxed for 1 h then partitioned between EtOA/sat. aq. NaHCO₃. Column chromatography on alumina (EtOAc) gave a product which was converted to the HCl salt to give 12 (24 mg, 13%). ¹H NMR (DMSO-d₆) δ ppm 10.25 (bs, 1H), 8.98 (bs, 2H), 8.61 (d, 1H, J=8.1 Hz), 8.33 (s, 1H), 8.09 (d, 1H, J=8.2 Hz), 8.00 (t, 1H, J=7.5 Hz), 7.89 (d, 1H, J=7.7 Hz), 7.82 (d, 1H, J=8.6 Hz), 7.72 (t, 1H, J=7.4 Hz), 7.58 (td, 1H, J=7.8, 0.9 Hz), 7.44 (t, 1H, J=7.6 Hz), 3.88-3.96 (m, 2H), 3.02-3.10 (m, 2H), 2.55 (t, 3H, J=5.4 Hz), 2.14 (p, 2H, J=7.2 Hz). ACPI-MS Found: [M+H]⁺=333.

### Example 4.13

N¹-[2-(1-benzofuran-2-yl)-4-quinazolinyl]-N-ethyl-1,3-propanediamine dihydrochloride (13). Mesyl chloride (55 μL, 0.70 mmol) was added to a solution of 3-{[2-(1-benzofuran-2-yl)-4-quinazolinyl]amino}-1-propanol (0.150 g, 0.470 mmol) and triethylamine (130 μL, 0.93 mmol) in thf (10 mL) at 0° C. in a sealed tube. The solution was stirred at 0° C. until consumption of starting material was evident by t.l.c. Ethylamine (310 μL, 4.74 mmol) was added and the solution was refluxed for 1 h then partitioned between EtOAc/sat. aq. NaHCO₃. Column chromatography on alumina (EtOAc) gave a product which was converted to the HCl salt to give 13 (105 mg, 53%). ¹H NMR (DMSO-d₆) δ ppm 9.9 (b, 1H), 8.81 (bs, 1H), 8.54 (d, 1H, J=7.7 Hz), 8.22 (bs, 1H), 7.92-8.06 (m, 2H), 7.88 (d, 1H, J=7.8 Hz), 7.81 (d, 1H, J=8.5 Hz), 7.70 (t, 1H, J=7.5 Hz), 7.56 (t, 1H, J=7.6 Hz), 7.43 (t, 1H, J=7.4 Hz), 3.86-3.94 (m, 2H), 3.02-3.10 (m, 2H), 2.89-2.98 (m, 2H), 2.13 (p, 2H, J=7.5 Hz), 1.20 (t, 3H, J=7.4 Hz). ACPI-MS Found: [M+H]⁺=347.

### Example 4.14

N¹-[2-(1-Benzofuran-2-yl)-4-quinazolinyl]-N³,N³,2,2-tetramethyl-1,3-propanediamine dihydrochloride (14). A solution of 2-(1-benzofuran-2-yl)-4-chloroquinazoline (H: R═H, R′=benzofuran-2-yl) (from 2-(1-benzofuran-2-yl)-4(3H)-quinazolinone; 0.472 g, 1.80 mmol) and N¹,N¹,2,2-tetramethyl-1,3-propanediamine (0.86 mL, 5.4 mmol) in dioxane (25 mL) was refluxed for 2 h, workup and conversion to the hydrochloride salt gave 14 (0.433 g, 54%) as a solid. ¹H NMR (DMSO-d₆) δ ppm 10.06 (bs, 1H), 9.78 (bs, 1H), 8.70 (d, 1H, J=8.1 Hz), 8.42 (bs, 1H), 8.07 (d, 1H, J=8.1 Hz), 8.00 (t, 1H, J=7.7 Hz), 7.89 (d, 1H, J=7.7 Hz), 7.82 (dd, 1H, J=8.4, 0.6 Hz), 7.72 (t, 1H, J=7.6 Hz), 7.58 (td, 1H, J=7.7, 0.8 Hz), 7.43 (t, 1H, J=7.4 Hz), 3.88 (d, 2H, J=6.1 Hz), 3.23 (d, 2H, J=4.8 Hz), 2.85 (d, 6H, J=4.8 Hz), 1.25 (s, 6H). ACPI-MS Found: [M+H]⁺=375.

### Example 4.15

N¹-[2-(1-Benzofuran-2-yl)pyrido[3,2-d]pyrimidin-4-yl]-N³,N³-dimethyl-1,3-propanediamine dihydrochloride (15). A mixture of 2-(1-benzofuran-2-yl)pyrido[3,2-o]pyrimidin-4(3H)-one (C: R=5-aza, R′=benzofuran-2-yl) (0.120 g, 0.456 mmol) and tetramethylammonium chloride (0.100 g, 0.912 mmol) in POCl₃ (10 mL) was refluxed for 2 h to give the chloropyridopyrimidine (H: R=5-aza, R′=benzofuran-2-yl). The chloropyridopyrimidine was refluxed with N¹,N¹-dimethyl-1,3-propanediamine (0.2 mL, 1.73 mmol) in dioxane (20 mL) for 2 h, workup and conversion to the hydrochloride salt gave 15 (0.152 g, 79%) as a solid. ¹H NMR (DMSO-d₈) δ ppm 10.18 (bs, 1H), 9.30 (bs, 1H), 8.86 (dd, 1H, J=4.3, 1.5 Hz), 8.31 (dd, 1H, J=8.5, 1.3 Hz), 8.02 (s, 1H), 7.93 (dd, 1H, J=8.5, 4.3 Hz), 7.83 (d, 1H, J=7.6 Hz), 7.78 (dd, 1H, J=8.3, 0.6 Hz), 7.50 (td, 1H, J=7.8, 1.2 Hz), 7.38 (td, 1H, J=7.5, 0.7 Hz), 3.78-3.84 (m, 2H), 3.16-3.23 (m, 2H), 2.77 (d, 6H, J=5.0 Hz), 2.10-2.20 (m, 2H). ACPI-MS Found: [M+H]⁺=348.

### Example 4.16

N¹-[2-(1-Benzofuran-2-yl)-5-methyl-4-quinazolinyl]-N³,N³-dimethyl-1,3-propanediamine dihydrochloride (16). A mixture of 2-(1-benzofuran-2-yl)-5-methyl-4(3H)-quinazolinone (C: R=5-Me, R′=benzofuran-2-yl) (0.240 g, 0.827 mmol) and tetramethylammonium chloride (0.181 g, 1.65 mmol) in POCl₃ (10 mL) was refluxed for 1 h to give the chloroquinazoline (H: R=5-Me, R′=benzofuran-2-yl). The chloroquinazoline was refluxed with N¹,N¹-dimethyl-1,3-propanediamine (0.3 mL, 2.60 mmol) in dioxane (40 mL) for 2 h, workup and conversion to the hydrochloride salt gave 16 (0.353 g, 98%) as a solid. ¹H NMR (DMSO-d₆) δ ppm 10.84 (bs, 1H), 8.63 (bs, 1H), 8.37 (s, 1H), 7.98 (d, 1H, J=8.3 Hz), 7.78-7.92 (m, 3H), 7.58 (td, 1H, J=7.8, 1.1 Hz), 7.50 (d, 1H, J=7.3 Hz), 7.43 (td, 1H, J=7.3, 0.6 Hz), 3.93-3.99 (m, 2H), 3.19-3.26 (m, 2H), 2.96 (s, 3H), 2.76 (d, 6H, J=5.0 Hz), 2.18-2.26 (m, 2H). ACPI-MS Found: [M+H]⁺=361.

### Example 4.17

N¹-[2-(1-benzofuran-2-yl)-5-methoxy-4-quinazolinyl]-N³,N³-dimethyl-1,3-propanediamine dihydrochloride (17). A mixture of 2-(1-benzofuran-2-yl)-5-methoxy-4(3H)-quinazolinone (C: R=5-OCH₃, R′=benzofuran-2-yl) (0.384 g, 1.31 mmol) and tetramethylammonium chloride (0.29 g, 2.6 mmol) in POCl₃ (10 mL) was refluxed for 0.5 h to give the chloroquinazoline (H: R=5-OCH₃, R′=benzofuran-2-yl). The chloroquinazoline was refluxed with N¹,N¹-dimethyl-1,3-propanediamine (0.50 mL, 4.0 mmol) in dioxane (50 mL) for 2 h, workup gave 17 (0.507 g, 86%) as a solid. ¹H NMR (DMSO-d₆) δ ppm 11.02 (bs, 1H), 9.72 (bs, 1H), 8.44 (s, 1H), 7.87-7.95 (m, 2H), 7.81 (dd, 1H, J=8.4, 0.6 Hz), 7.72 (bd, 1H, J=8.3 Hz), 7.59 (td, 1H, J=7.3, 1.2 Hz), 7.44 (td, 1H, J=7.5, 0.7 Hz), 7.26 (d, 1H, J=8.2 Hz), 4.12 (s, 3H), 3.89-3.97 (m, 2H), 3.15-3.22 (m, 2H), 2.75 (d, 6H, J=4.9 Hz), 2.15-2.24 (m, 2H). ACPI-MS Found: [M+H]⁺=377.

### Example 4.18

N¹-[2-(1-Benzofuran-2-yl)-5-chloro-4-quinazolinyl]-N³,N³-dimethyl-1,3-propanediamine dihydrochloride (18). A mixture of crude 2-(1-benzofuran-2-yl)-5-chloro-4(3H)-quinazolinone (C: R=5-Cl, R′=benzofuran-2-yl) and tetramethylammonium chloride (0.680 g, 6.20 mmol) in POCl₃ (20 mL) was refluxed for 1 h to give the chloroquinazoline (H: R=5-Cl, R′=benzofuran-2-yl). The chloroquinazoline was refluxed with N¹,N¹-dimethyl-1,3-propanediamine (1 mL, 8.7 mmol) in dioxane (60 mL) for 2 h, workup and conversion to the hydrochloride salt gave 18 (0.944 g, 67%) as a solid. ¹H NMR (DMSO-d₆) δ ppm 10.48 (bs, 1H), 8.76 (bs, 1H), 7.93 (s, 1H), 7.80-7.86 (m, 2H), 7.77 (dd, 1H, J=8.4, 0.6 Hz), 7.67 (dd, 1H, J=7.7, 1.1 Hz), 7.51 (ddd, 1H, J=8.3, 7.3, 1.2 Hz), 7.38 (td, 1H, J=7.5, 0.7 Hz), 3.85-3.92 (m, 2H), 3.16-3.23 (m, 2H), 2.77 (d, 6H, J=5.0 Hz), 2.15-2.23 (m, 2H). ACPI-MS Found: [M+H]⁺=381, 383.

### Example 4.19

N¹-[2-(1-Benzofuran-2-yl)-4-nitro-4-quinazolinyl]-N³,N³-dimethyl-1,3-propanediamine (19). A mixture of 2-(1-benzofuran-2-yl)-5-nitro-4(3H)-quinazolinone (C: R=5-NO₂, R′=benzofuran-2-yl) (0.430 g, 1.40 mmol) and tetramethylammonium chloride (0.30 g, 2.74 mmol) in POCl₃ (15 mL) was refluxed for 0.5 h to give the chloroquinazoline (H: R=5-NO₂, R′=benzofuran-2-yl). The chloroquinazoline was refluxed with N¹,N¹-dimethyl-1,3-propanediamine (0.5 mL, 4.3 mmol) in dioxane (50 mL) for 2 h, workup gave 19 (0.128 g, 23%) as a solid. ¹H NMR (DMSO-d₆) δ ppm 8.10 (d, 1H, J=8.3 Hz), 8.06 (d, 1H, J=7.6 Hz), 7.99 (bs, 1H), 7.91 (t, 1H, J=8.0 Hz), 7.76-7.81 (m, 2H), 7.73 (dd, 1H, J=8.3, 0.7 Hz), 7.44 (ddd, 1H, J=7.6, 7.3, 1.2 Hz), 7.33 (td, 1H, J=7.5, 0.8 Hz), 3.67-3.72 (m, 2H), 2.41 (t, 2H, J=6.4 Hz), 2.19 (s, 6H), 1.77-1.85 (m, 2H). ACPI-MS Found: [M+H]⁺=392.

### Example 4.20

N¹-[2-(1-Benzofuran-2-yl)-N⁴-[3-(dimethylamino)propyl]-4,5-quinazolinediamine dihydrochloride (20). A solution of N¹-[2-(1-benzofuran-2-yl)-5-nitro-4-quinazolinyl]-N³,N³-dimethyl-1,3-propanediamine (19) (0.088 g, 0.225 mmol) and 5% Pd on carbon (20 mg) in methanol (30 mL) was hydrogenated (40 p.s.i.) for 17 h. The solution was filtered and the solvent removed in vacuo, conversion to the hydrochloride salt gave 20 (0.102 g, 98%) as a solid. ¹H NMR (DMSO-d₆) δ ppm 14.4 (b, 1H), 10.52 (bs, 1H), 8.38 (s, 1H), 7.92 (d, 1H, J=7.8 Hz), 7.83 (dd, 1H, J=8.5, 0.7 Hz), 7.68 (t, 1H, J=8.1 Hz), 7.61 (td, 1H, J=7.8, 1.1 Hz), 7.46 (td, 1H, J=7.5, 0.6 Hz), 7.39 (d, 1H, J=7.6 Hz), 7.08 (dd, 1H, J=8.1, 0.7 Hz), 3.90 (t, 2H, J=6.6 Hz), 3.18-3.25 (m, 2H), 2.76 (d, 6H, J=4.9 Hz), 2.12-2.21 (m, 2H). ACPI-MS Found: [M+H]⁺=362.

### Example 4.21

2-(1-benzofuran-2-yl)-N-[3-(dimethylamino)propyl]-4-{[3-(dimethylamino)propyl]amino}-5-quinazolinecarboxamide (21). A mixture of 2-(1-benzofuran-2-yl)-4-oxo-3,4-dihydro-5-quinazolinecarboxamide (C: R=5-CONH₂, R′=1-benzofuran-2-yl) (0.141 g, 0.462 mmol) and tetramethylammonium chloride (0.10 g, 0.922 mmol) in POCl₃ (5 mL) was refluxed for 0.5 h to give the chloroquinazoline (H: R=5-CN, R′=benzofuran-2-yl). The chloroquinazoline was refluxed with N¹,N¹-dimethyl-1,3-propanediamine (0.18 mL, 1.4 mmol) in dioxane (10 mL) for 2 h, workup gave 21 (0.180 g, 82%) as a solid. ¹H NMR (DMSO-d₆) δ ppm 9.09 (t, 1H, J=5.5 Hz), 8.50 (t, 1H, J=4.9 Hz), 7.91 (dd, 1H, J=8.4, 1.3 Hz), 7.76-7.88 (m, 2H), 7.70-7.75 (m, 2H), 7.50 (dd, 1H, J=7.2, 1.3 Hz), 7.42 (ddd, 1H, J=8.4, 7.3, 1.2 Hz), 7.32 (td, 1H, J=7.6, 0.8 Hz), 3.60-3.67 (m, 2H), 3.34-3.41 (m, 2H), 2.37 (t, 2H, J=7.0 Hz), 2.30 (t, 2H, J=7.0 Hz), 2.18 (s, 6H), 2.15 (s, 6H), 1.76-1.84 (m, 2H), 1.68-1.75 (m, 2H). ACPI-MS Found: [M+H]⁺=476.

### Example 4.22

N¹-[2-(1-Benzofuran-2-yl)pyrido[4,3-d]pyrimidin-4-yl]-N³,N³-dimethyl-1,3-propanediamine (22). A mixture of 2-(1-benzofuran-2-yl)pyrido[4,3-d]pyrimidin-4(3H)-one (C: R=6-aza, R′=benzofuran-2-yl) (0.211 g, 0.801 mmol) and tetramethylammonium chloride (0.20 g, 1.82 mmol) in POCl₃ (15 mL) was refluxed for 2 h to give the chloropyridopyrimidine (H: R=6-aza, R′=benzofuran-2-yl). The chloropyridopyrimidine was refluxed with N¹,N¹-dimethyl-1,3-propanediamine (0.3 mL, 2.60 mmol) in dioxane (50 mL) for 3 h, workup gave 22 (0.154 g, 55%) as a solid. ¹H NMR (DMSO-d₆) δ ppm 9.52 (d, 1H, J=0.5 Hz), 8.94 (t, 1H, J=5.3 Hz), 8.73 (d, 1H, J=5.7 Hz), 7.77-7.82 (m, 2H), 7.72 (dd, 1H, J=8.3, 0.8 Hz), 7.62 (dd, 1H, J=5.8, 0.6 Hz), 7.45 (ddd, 1H, J=8.3, 7.3, 1.3 Hz), 7.33 (td, 1H, J=7.5, 0.9 Hz), 3.67-3.75 (m, 2H), 2.39 (t, 2H, J=6.9 Hz), 2.20 (s, 6H), 1.84-1.92 (m, 2H). ACPI-MS Found: [M+H]⁺=348.

### Example 4.23

N¹-[2-(1-Benzofuran-2-yl)-6-methyl-4-quinazolinyl]-N³,N³-dimethyl-1,3-propanediamine dihydrochloride (23). A mixture of 2-(1-benzofuran-2-yl)-6-methyl-4(3H)-quinazolinone (C: R=6-Me, R′=benzofuran-2-yl) (0.248 g, 0.898 mmol) in thionyl chloride (10 mL) was refluxed for 10 min to give the chloroquinazoline (H: R=6-Me, R′=benzofuran-2-yl). The chloroquinazoline was refluxed with N¹,N¹-dimethyl-1,3-propanediamine (0.34 mL, 2.7 mmol) in dioxane (40 mL) for 2 h, workup and conversion to the hydrochloride salt gave 23 (0.239 g, 61%) as a solid. ¹H NMR (DMSO-d₆) δ ppm 10.23 (bs, 1H), 8.75 (bs, 1H), 8.17 (bd, 1H, J=8.2 Hz), 7.85 (s, 1H), 7.79 (d, 1H, J=7.4 Hz), 7.75 (dd, 1H, J=8.2, 0.7 Hz), 7.70 (d, 1H, J=7.1 Hz), 7.44 (t, 1H, J=7.4 Hz), 7.34 (td, 1H, J=7.4, 0.8 Hz), 3.72-3.80 (m, 2H), 3.18-3.25 (m, 2H), 2.78 (d, 6H, J=5.0 Hz), 2.69 (s, 3H), 2.11-2.20 (m, 2H). ACPI-MS Found: [M+H]⁺=361.

### Example 4.24

N¹-[2-(1-Benzofuran-2-yl)-6-(trifluoromethyl)-4-quinazolinyl]-N³,N³-dimethyl-1,3-propanediamine (24). A mixture of 2-(1-benzofuran-2-yl)-6-(trifluoromethyl)-4(3H)-quinazolinone (C: R=6-CF₃, R′=benzofuran-2-yl) (0.130 g, 0.394 mmol) and tetramethylammonium chloride (0.090 g, 0.821 mmol) in POCl₃ (10 mL) was refluxed for 0.5 h to give the chloroquinazoline (H: R=6-CF₃, R′=benzofuran-2-yl). The chloroquinazoline was refluxed with N¹N¹-dimethyl-1,3-propanediamine (0.14 mL, 1.1 mmol) in dioxane (20 mL) for 2 h, workup gave 24 (0.152 g, 93%) as a solid. ¹H NMR (DMSO-d₆) δ ppm 8.84 (t, 1H, J=5.2 Hz), 8.74 (s, 1H), 8.04 (dd, 1H, J=8.8, 1.9 Hz), 7.94 (d, 1H, J=8.7 Hz), 7.75-7.82 (m, 2H), 7.72 (dd, 1H, J=8.3, 0.7 Hz), 7.44 (ddd, 1H, J=8.2, 7.2, 1.3 Hz), 7.33 (td, 1H, J=7.5, 0.8 Hz), 3.67-3.74 (m, 2H), 2.39 (t, 2H, J=6.9 Hz), 2.20 (s, 6H), 1.84-1.93 (m, 2H). ACPI-MS Found: [M+H]⁺=415.

### Example 4.25

N¹-[2-(1-Benzofuran-2-yl)-6-methoxy-4-quinazolinyl]-NR³,N³-dimethyl-1,3-propanediamine dihydrochloride (25). A mixture of 2-(1-benzofuran-2-yl)-6-methoxy-4(3H)-quinazolinone (C: R=6-OMe, R′=benzofuran-2-yl) (0.257 g, 0.879 mmol) and tetramethylammonium chloride (0.200 g, 1.82 mmol) in POCl₃ (15 mL) was refluxed for 45 min to give the chloroquinazoline (H: R=6-OMe R′=benzofuran-2-yl). The chloroquinazoline was refluxed with N¹N¹-dimethyl-1,3-propanediamine (0.30 mL, 2.38 mmol) in dioxane (20 mL) for 2 h, workup and conversion to the hydrochloride salt gave 25 (0.314 g, 79%) as a solid. ¹H NMR (DMSO-d₆) δ ppm 15.0 (bs, 1H), 10.53 (bs, 1H), 10.32 (bs, 1H), 8.15-8.36 (m, 2H), 8.06 (d, 1H, J=9.1 Hz), 7.88 (d, 1H, J=7.7 Hz), 7.80 (dd, 1H, J=8.4, 0.5 Hz), 7.64 (dd, 1H, J=9.1, 2.5 Hz), 7.57 (td, 1H, J=7.8, 0.9 Hz), 7.43 (t, 1H, J=7.5 Hz), 3.97 (s, 3H), 3.85-3.92 (2H, m), 3.20-3.28 (2H, m), 2.77 (d, 6H, J=4.9 Hz), 2.16-2.26 (m, 2H). ACPI-MS Found: [M+H]⁺=377.

### Example 4.26

N¹-[2-(1-benzofuran-2-yl)-6-fluoro-4-quinazolinyl]-N³,N³-dimethyl-1,3-propanediamine (26). A mixture of 2-(1-benzofuran-2-yl)-6-fluoro-4(3H)-quinazolinone (C: R=6-F, R′=benzofuran-2-yl) (0.206 g, 0.735 mmol) and tetramethylammonium chloride (0.16 g, 1.46 mmol) in POCl₃ (5 mL) was refluxed for 0.5 h to give the chloroquinazoline (H: R=6-F, R′=benzofuran-2-yl). The chloroquinazoline was refluxed with N¹,N¹-dimethyl-1,3-propanediamine (0.30 mL, 2.4 mmol) in dioxane (50 mL) for 2 h, workup gave 26 (0.150 g, 40%) as a solid. ¹H NMR (DMSO-d₆) δ ppm 8.37 (t, 1H, J=5.3 Hz), 8.11 (dd, 1H, J=9.9, 2.8 Hz), 7.87 (dd, 1H, J=9.2, 5.5 Hz), 7.77 (d, 1H, J=7.3 Hz), 7.66-7.73 (m, 3H), 7.41 (td, 1H, J=7.3, 1.3 Hz), 7.31 (td, 1H, J=7.4, 0.9 Hz), 3.65-3.72 (m, 2H), 2.38 (t, 2H, J=7.0 Hz), 2.19 (s, 6H), 1.84-1.91 (m, 2H). ACPI-MS Found: [M+H]⁺=365.

### Example 4.27

N¹-[2-(1-Benzofuran-2-yl)-6-chloro-4-quinazolinyl]-N³,N³-dimethyl-1,3-propanediamine dihydrochloride (27). A mixture of 2-(1-benzofuran-2-yl)-6-chloro-4(3H)-quinazolinone (C: R=6-Cl, R′=benzofuran-2-yl) (0.722 g, 2.43 mmol) and tetramethylammonium chloride (0.533 g, 4.86 mmol) in POCl₃ (20 mL) was refluxed for 0.5 h to give the chloroquinazoline (H: R=6-Cl, R′=benzofuran-2-yl). The chloroquinazoline was refluxed with N¹,N¹-dimethyl-1,3-propanediamine (0.84 mL, 6.68 mmol) in dioxane (80 mL) for 2 h, workup and conversion to the hydrochloride salt gave 27 (0.622 g, 56%) as a solid. ¹H NMR (DMSO-d₆) δ ppm 10.66 (bs, 1H), 10.13 (bs, 1H), 8.79 (d, 1H, J=1.7 Hz), 8.28 (s, 1H), 8.08 (d, 1H, J=8.9 Hz), 7.99 (dd, 1H, J=8.9, 2.1 Hz), 7.86 (d, 1H, J=7.6 Hz), 7.79 (dd, 1H, J=8.3, 0.6 Hz), 7.55 (ddd, 1H, J=8.3, 7.3, 1.2 Hz), 7.41 (td, 1H, J=7.5, 0.7 Hz), 3.81-3.89 (m, 2H), 3.19-3.26 (m, 2H), 2.76 (d, 6H, J=4.9 Hz), 2.14-2.23 (m, 2H). ACPI-MS Found: [M+H]⁺=381, 383.

### Example 4.28

N¹-[2-(1-benzofuran-2-yl)-6-bromo-4-quinazolinyl]-N³,N³-dimethyl-1,3-propanediamine dihydrochloride (28). A mixture of 2-(1-benzofuran-2-yl)-6-bromo-4(3H)-quinazolinone (C: R=6-Br, R′=benzofuran-2-yl) (0.514 g, 1.51 mmol) and tetramethylammonium chloride (0.321 g, 2.93 mmol) in POCl₃ (20 mL) was refluxed for 0.5 h to give the chloroquinazoline (H: R=6-Br, R′=benzofuran-2-yl). The chloroquinazoline was refluxed with N¹,N¹-dimethyl-1,3-propanediamine (0.60 mL, 4.8 mmol) in dioxane (100 mL) for 2 h, workup and conversion to the hydrochloride salt gave 28 (0.464 g, 62%) as a solid. ¹H NMR (DMSO-d₆) δ ppm 10.19 (bs, 1H), 9.47 (bs, 1H), 8.74 (bs, 1H), 8.02-8.10 (m, 2H), 7.89 (d, 1H, J=8.9 Hz), 7.84 (d, 1H, J=7.7 Hz), 7.77 (dd, 1H, J=8.4, 0.6 Hz), 7.52 (td, 1H, J=7.8, 1.1 Hz), 7.39 (td, 1H, J=7.7, 0.4 Hz), 3.77-4.05 (m, 2H), 3.19-3.26 (m, 2H), 2.78 (d, 6H, J=4.9 Hz), 2.11-2.20 (m, 2H). ACPI-MS Found: [M+H]⁺=427, 425.

### Example 4.29

N¹-[2-(1-Benzofuran-2-yl)-6-nitro-4-quinazolinyl]-N³,N³-dimethyl-1,3-propanediamine (29). A mixture of 2-(1-benzofuran-2-yl)-6-nitro-4(3H)-quinazolinone (C: R=6-NO₂, R′=benzofuran-2-yl) (1.031 g, 3.36 mmol) and tetramethylammonium chloride (0.74 g, 6.75 mmol) in POCl₃ (40 mL) was refluxed for 1 h to give the chloroquinazoline (H: R=6-NO₂, R′=benzofuran-2-yl) (0.95 g, 87%). The chloroquinazoline (0.220 g, 0.675 mmol) was refluxed with N¹,N¹-dimethyl-1,3-propanediamine (0.25 mL, 2.0 mmol) in dioxane (30 mL) for 2 h, workup gave 29 (0.256 g, 97%) as a solid. ¹H NMR (DMSO-d₆) δ ppm 9.33 (d, 1H, J=2.5 Hz), 9.15 (bs, 1H), 8.49 (dd, 1H, J=9.2, 2.5 Hz), 7.92 (d, 1H, J=9.2 Hz), 7.83 (d, 1H, J=0.9 Hz), 7.81 (dd, 1H, J=7.1, 0.7 Hz), 7.73 (dd, 1H, J=8.3, 0.7 Hz), 7.45 (ddd, 1H, J=8.3, 7.5, 1.3 Hz), 7.34 (td, 1H, J=7.5, 0.9 Hz), 3.68-3.76 (m, 2H), 2.39 (t, 2H, J=6.9 Hz), 2.21 (s, 6H), 1.89 (tt, 2H, J=7.2, 6.9 Hz). ACPI-MS Found: [M+H]⁺=392.

### Example 4.30

N¹-[2-(1-Benzofuran-2-yl)-N⁴-[3-(dimethylamino)propyl]-4,6-quinazolinediamine dihydrochloride (30). A solution of N¹-[2-(1-benzofuran-2-yl)-6-nitro-4-quinazolinyl]-N³,N³-dimethyl-1,3-propanediamine (30) (0.107 g, 0.273 mmol) and 5% Pd on carbon (20 mg) in methanol (50 mL) was hydrogenated (60 p.s.i.) for 3 h. The solution was filtered and the solvent removed in vacuo, conversion to the hydrochloride salt gave 30 (79 mg, 61%) as a solid. ¹H NMR (DMSO-d₆) δ ppm 14.5 (br, 1H), 10.61 (bs, 1H), 9.83 (bs, 1H), 8.28 (s, 1H), 7.87-7.94 (m, 2H), 7.81 (dd, 1H, J=8.4, 0.6 Hz), 7.58 (ddd, 1H, J=8.4, 7.3, 1.2 Hz), 7.44 (td, 1H, J=7.5, 0.7 Hz), 7.33-7.40 (m, 2H), 6.0 (br, 2H), 3.80-3.88 (m, 2H), 3.16-3.25 (m, 2H), 2.75 (d, 6H, J=5.0 Hz), 2.11-2.21 (m, 2H). ACPI-MS Found: [M+H]⁺=362.

### Example 4.31

2-(1-benzofuran-2-yl)-4-{[3-(dimethylamino)propyl]amino}-6-quinazolinecarbonitrile (31). A mixture of 2-(1-benzofuran-2-yl)-4-oxo-3,4-dihydro-6-quinazolinecarboxamide (C: R=6-CONH₂, R′=benzofuran-2-yl) (0.328 g, 1.08 mmol) and tetramethylammonium chloride (0.24 g, 2.2 mmol) in POCl₃ (10 mL) was refluxed for 0.5 h to give the chloroquinazoline (H: R=6-CN, R′=benzofuran-2-yl). The chloroquinazoline was refluxed with N¹,N¹-dimethyl-1,3-propanediamine (0.40 mL, 3.2 mmol) in dioxane (30 mL) for 1 h, workup gave 31 (0.208 g, 52%) as a solid. ¹H NMR (DMSO-d₆) δ ppm 8.84 (d, 1H, J=1.6 Hz), 8.71 (t, 1H, J=5.3 Hz), 8.09 (dd, 1H, J=8.7, 1.8 Hz), 7.88 (d, 1H, J=8.7 Hz), 7.77-7.82 (m, 2H), 7.73 (dd, 1H, J=8.3, 0.7 Hz), 7.44 (ddd, 1H, J=8.4, 7.3, 1.3 Hz), 7.33 (td, 1H, J=7.5, 0.8 Hz), 3.65-3.73 (m, 2H), 2.38 (t, 2H, J=7.0 Hz), 2.19 (s, 6H), 1.84-1.93 (m, 2H). ACPI-MS Found: [M+H]⁺=372.

### Example 4.32

2-(1-benzofuran-2-yl)-4-{[3-(dimethylamino)propyl]amino}-6-quinazolinecarboxamide dihydrochloride (32). A mixture of 2-(1-benzofuran-2-yl)-4-{[3-(dimethylamino)propyl]amino}-6-quinazolinecarbonitrile (31) (31.5 mg, 0.085 mmol) and KOH (0.068 g, 1.21 mmol) in t-butanol (3 mL) was refluxed in a sealed tube for 1 h. The mixture was quenched with brine (10 mL), extracted into EtOAc and washed with water. Removal of the solvent in vacuo gave an oil, conversion to the hydrochloride salt gave the product 32 (22 mg, 56%) as a solid. ¹H NMR (DMSO-d₆) δ ppm 9.97 (bs, 1H), 9.55 (bs, 1H), 9.14 (s, 1H), 8.32 (dd, 1H, J=8.7, 1.5 Hz), 8.15 (bs, 1H), 8.08 (bs, 1H), 7.95 (d, 1H, J=8.0 Hz), 7.85 (d, 1H, J=7.6 Hz), 7.79 (d, 1H, J=8.4 Hz), 7.65 (bs, 1H), 7.52 (t, 1H, J=7.3 Hz), 7.39 (t, 1H, J=7.3 Hz), 3.79-3.88 (m, 2H), 3.21-3.30 (m, 2H), 2.79 (d, 6H, J=4.9 Hz), 2.12-2.21 (m, 2H). ACPI-MS Found: [M+H]⁺=390.

### Example 4.33

N¹-[2-(1-Benzofuran-2-yl)pyrido[3,4-a]pyrimidin-4-yl]-N³,N³-dimethyl-1,3-propanediamine dihydrochloride (33). A mixture of 2-(1-benzofuran-2-yl)pyrido[3,4-d]pyrimidin-4(3H)-one (C: R=7-aza, R′=benzofuran-2-yl) (0.176 g, 0.669 mmol) and tetramethylammonium chloride (0.150 g, 1.37 mmol) in POCl₃ (15 mL) was refluxed for 2 h to give the chloropyridopyrimidine (H: R=7-aza, R′=benzofuran-2-yl). The chloropyridopyrimidine was refluxed with N¹,N¹-dimethyl-1,3-propanediamine (0.23 mL, 1.83 mmol) in dioxane (20 mL) for 2 h, workup and conversion to the hydrochloride salt gave 33 (0.159 g, 57%) as a solid. ¹H NMR (DMSO-d₆) δ ppm 10.29 (s, 1H), 9.49 (s, 1H), 9.20 (s, 1H), 8.70 (d, 1H, J=5.6 Hz), 8.37 (d, 1H, J=5.4 Hz), 7.99 (s, 1H), 7.82 (d, 1H, J=7.5 Hz), 7.77 (dd, 1H, J=8.3, 0.7 Hz), 7.49 (td, 1H, J=7.8, 1.2 Hz), 7.37 (td, 1H, J=7.5, 0.8 Hz), 3.75-3.82 (m, 2H), 3.18-3.27 (m, 2H), 2.78 (d, 6H, J=5.0 Hz), 2.13-2.22 (m, 2H). ACPI-MS Found: [M+H]⁺=348.

### Example 4.34

N¹-[2-(1-benzofuran-2-yl)-7-methyl-4-quinazolinyl]-N³,N³-dimethyl-1,3-propanediamine dihydrochloride (34). A mixture of 2-(1-benzofuran-2-yl)-7-methyl-4(3H)-quinazolinone (C: R=7-CH₃, R′=benzofuran-2-yl) (0.251 g, 0.908 mmol) and tetramethylammonium chloride (0.20 g, 1.8 mmol) in POCl₃ (10 mL) was refluxed for 0.5 h to give the chloroquinazoline (H: R=7-CH₃, R′=benzofuran-2-yl). The chloroquinazoline was refluxed with N¹,N¹-dimethyl-1,3-propanediamine (0.35 mL, 2.8 mmol) in dioxane (50 mL) for 2 h, workup and conversion to the hydrochloride salt gave 34 (0.337 g, 86%) as a solid. ¹H NMR (DMSO-d₆) δ ppm 10.47 (bs, 1H), 10.10 (bs, 1H), 8.47 (d, 1H, J=8.4 Hz), 8.31 (s, 1H), 7.84-7.91 (m, 2H), 7.81 (dd, 1H, J=8.4, 0.5 Hz), 7.53-7.61 (m, 2H), 7.44 (t, 1H, J=7.2 Hz), 3.82-3.91 (m, 2H), 3.19-3.27 (m, 2H), 2.77 (d, 6H, J=4.9 Hz), 2.54 (s, 3H), 2.12-2.22 (m, 2H). ACPI-MS Found: [M+H]⁺=361.

### Example 4.35

N¹-[2-(1-Benzofuran-2-yl)-7-(trifluoromethyl)-4-quinazolinyl]-N³,N³-dimethyl-1,3-propanediamine dihydrochloride (35). A mixture of 2-(1-benzofuran-2-yl)-7-(trifluoromethyl)-4(3H)-quinazolinone (C: R=7-CF₃, R′=benzofuran-2-yl) (0.261 g, 0.790 mmol) and tetramethylammonium chloride (0.175 g, 1.60 mmol) in POCl₃ (10 mL) was refluxed for 20 min to give the chloroquinazoline. The chloroquinazoline (H: R=7-CF₃, R′=benzofuran-2-yl) was refluxed with N¹,N¹-dimethyl-1,3-propanediamine (0.30 mL, 2.4 mmol) in dioxane (40 mL) for 2 h, workup and conversion to the hydrochloride salt gave 35 (0.302 g, 78%) as a solid. ¹H NMR (DMSO-d₆) δ ppm 10.44 (s, 1H), 9.81 (s, 1H), 8.73 (d, 1H, J=8.6 Hz), 8.30 (s, 1H), 8.14 (s, 1H), 7.95 (dd, 1H, J=8.6, 1.3 Hz), 7.85 (d, 1H, J=7.6 Hz), 7.78 (dd, 1H, J=8.4, 0.7 Hz), 7.52 (td, 1H, J=7.8, 1.2 Hz), 7.39 (td, 1H, J=7.6, 0.7 Hz), 3.80-3.90 (m, 2H), 3.20-3.27 (m, 2H), 2.77 (d, 6H, J=5.0 Hz), 2.16-2.24 (m, 2H). ACPI-MS Found: [M+H]⁺=415.

### Example 4.36

N¹-[2-(1-Benzofuran-2-yl)-7-methoxy-4-quinazolinyl]-N³,N³-dimethyl-1,3-propanediamine dihydrochloride (36). A mixture of 2-(1-benzofuran-2-yl)-7-methoxy-4(3H)-quinazolinone (C: R=7-OMe, R′=benzofuran-2-yl) (0.258 g, 0.883 mmol) and tetramethylammonium chloride (0.200 g, 1.82 mmol) in POCl₃ (15 mL) was refluxed for 30 min to give the chloroquinazoline (H: R=7-OMe, R′=benzofuran-2-yl). The chloroquinazoline was refluxed with N¹,N¹-dimethyl-1,3-propanediamine (0.31 mL, 2.46 mmol) in dioxane (40 mL) for 2 h, workup and conversion to the hydrochloride salt gave 36 (0.317 g, 80%) as a solid. ¹H NMR (DMSO-d₆) δ ppm 14.5 (bs, 1H), 10.64 (bs, 1H), 10.20 (bs, 1H), 8.57 (d, 1H, J=9.2 Hz), 8.38 (s, 1H), 7.90 (d, 1H, J=7.7 Hz), 7.80 (dd, 1H, J=8.4, 0.6 Hz), 7.55-7.64 (m, 2H), 7.44 (td, 1H, J=7.5, 0.4 Hz), 7.35 (dd, 1H, J=9.1, 2.4 Hz), 3.96 (s, 3H), 3.82-3.90 (m, 2H), 3.20-3.27 (m, 2H), 2.76 (d, 6H, J=5.0 Hz), 2.14-2.24 (m, 2H). ACPI-MS Found: [M+H]⁺=377.

### Example 4.37

N¹-[2-(1-benzofuran-2-yl)-7-fluoro-4-quinazolinyl]-N³,N³-dimethyl-1,3-propanediamine dihydrochloride (37). A mixture of 2-(1-Benzofuran-2-yl)-7-fluoro-4(3H)-quinazolinone (C: R=7-F, R′=1-benzofuran-2-yl) (0.265 g, 0.946 mmol) and tetramethylammonium chloride (0.21 g, 1.46 mmol) in POCl₃ (6 mL) was refluxed for 0.5 h to give the chloroquinazoline (H: R=7-F, R′=benzofuran-2-yl). The chloroquinazoline was refluxed with N¹,N¹-dimethyl-1,3-propanediamine (0.36 mL, 2.9 mmol) in dioxane (50 mL) for 2 h, workup gave 37 (0.365 g, 88%) as a solid. ¹H NMR (DMSO-d₆) δ ppm 10.49 (bs, 1H), 9.99 (bs, 1H), 8.64-8.71 (m, 1H), 8.23 (s, 1H), 7.87 (d, 1H, J=7.7 Hz), 7.75-7.83 (m, 2H), 7.51-7.64 (m, 2H), 7.41 (td, 1H, J=7.5, 0.6 Hz), 3.82-3.89 (m, 2H), 3.19-3.27 (m, 2H), 2.77 (d, 6H, J=4.9 Hz), 2.13-2.23 (m, 2H). ACPI-MS Found: [M+H]⁺=365.

### Example 4.38

N¹-[2-(1-Benzofuran-2-yl)-7-chloro-4-quinazolinyl]-N³,N³-dimethyl-1,3-propanediamine dihydrochloride (38). A mixture of 2-(1-benzofuran-2-yl)-7-chloro-4(3H)-quinazolinone (C: R=7-Cl, R′=1-benzofuran-2-yl) (0.480 g, 1.62 mmol) and tetramethylammonium chloride (0.355 g, 3.24 mmol) in POCl₃ (10 mL) was refluxed for 30 min to give the chloroquinazoline (H: R=7-Cl, R′=benzofuran-2-yl). The chloroquinazoline was refluxed with N¹,N¹-dimethyl-1,3-propanediamine (0.56 mL, 4.45 mmol) in dioxane (40 mL) for 2 h, workup and conversion to the hydrochloride salt gave 38 (0.558 g, 76%) as a solid. ¹H NMR (DMSO-d₆) δ ppm 10.63 (bs, 1H), 10.19 (bs, 1H), 8.65 (d, 1H, J=8.9 Hz), 8.30 (s, 1H), 8.13 (d, 1H, J=1.8 Hz), 7.87 (d, 1H, J=7.6 Hz), 7.81 (dd, 1H, J=8.4, 0.7 Hz), 7.74 (dd, 1H, J=8.8, 2.0 Hz), 7.56 (ddd, 1H, J=8.3, 7.2, 1.3 Hz), 7.41 (td, 1H, J=7.5, 0.7 Hz), 3.83-3.88 (m, 2H), 3.20-3.27 (m, 2H), 2.76 (d, 6H, J=4.9 Hz), 2.15-2.24 (m, 2H). ACPI-MS Found: [M+H]⁺=381, 383.

### Example 4.39

N¹-[2-(1-benzofuran-2-yl)-7-bromo-4-quinazolinyl]-N³,N³-dimethyl-1,3-propanediamine dihydrochloride (39). A mixture of 2-(1-benzofuran-2-yl)-7-bromo-4(3H)-quinazolinone (C: R=7-Br, R′=1-benzofuran-2-yl) (0.440 g, 1.29 mmol) and tetramethylammonium chloride (0.28 g, 2.55 mmol) in POCl₃ (15 mL) was refluxed for 0.5 h to give the chloroquinazoline (H: R=7-Br, R′=benzofuran-2-yl). The chloroquinazoline was refluxed with N¹,N¹-dimethyl-1,3-propanediamine (0.49 mL, 3.9 mmol) in dioxane (50 mL) for 2 h, workup and conversion to the hydrochloride salt gave 39 (0.545 g, 85%) as a solid. ¹H NMR (DMSO-d₆) δ ppm 10.47 (bs, 1H), 9.92 (bs, 1H), 8.49 (d, 1H, J=8.8 Hz), 8.22 (s, 2H), 7.82-7.89 (m, 2H), 7.79 (d, 1H, J=8.4 Hz), 7.54 (td, 1H, J=7.8, 1.0 Hz), 7.41 (t, 1H, J=7.8 Hz), 3.80-3.88 (m, 2H), 3.19-3.26 (m, 2H), 2.77 (d, 6H, J=4.9 Hz), 2.12 (m, 2H). ACPI-MS Found: [M+H]⁺=427, 425.

### Example 4.40

N¹-[2-(1-Benzofuran-2-yl)-7-nitro-4-quinazolinyl]-N³,N³-dimethyl-1,3-propanediamine (40). A mixture of 2-(1-benzofuran-2-yl)-7-nitro-4(3H)-quinazolinone (C: R=7-NO₂, R′=1-benzofuran-2-yl) (0.505 g, 1.64 mmol) and tetramethylammonium chloride (0.360 g, 3.28 mmol) in POCl₃ (20 mL) was refluxed for 30 min to give the chloroquinazoline (H: R=7-NO₂, R′=benzofuran-2-yl). The chloroquinazoline was refluxed with N¹,N¹-dimethyl-1,3-propanediamine (0.60 mL, 4.77 mmol) in dioxane (40 mL) for 2 h, workup gave 40 (0.248 g, 39%) as a solid. ¹H NMR (DMSO-d₈) 8 ppm 8.88 (t, 1H, J=5.2 Hz), 8.45-8.50 (m, 2H), 8.25 (dd, 1H, J=9.0, 2.4 Hz), 7.76-7.82 (m, 2H), 7.73 (dd, 1H, J=8.3, 0.7 Hz), 7.44 (ddd, 1H, J=8.3, 7.3, 1.4 Hz), 7.33 (td, 1H, J=7.5, 0.9 Hz), 3.67-3.75 (m, 2H), 2.39 (t, 2H, J=6.9 Hz), 2.20 (s, 6H), 1.84-1.92 (m, 2H). ACPI-MS Found: [M+H]⁺=392.

### Example 4.41

N¹-[2-(1-Benzofuran-2-yl)-7-amino-4-quinazolinyl]-N³,N³-dimethyl-1,3-propanediamine dihydrochloride (41). A solution of N¹-[2-(1-benzofuran-2-yl)-7-nitro-4-quinazolinyl]-N³,N³-dimethyl-1,3-propanediamine (40) (0.075 g, 0.192 mmol) and 5% Pd on carbon (20 mg) in methanol (30 mL) was hydrogenated (40 p.s.i.) for 3 h. The solution was filtered and the solvent removed in vacuo, conversion to the hydrochloride salt gave 41 (64 mg, 77%) as a solid. ¹H NMR (DMSO-d₆) δ ppm 13.99 (bs, 1H), 10.16 (bs, 1H), 9.67 (bs, 1H), 8.27 (bs, 1H), 8.15 (d, 1H, J=9.0 Hz), 7.90 (d, 1H, J=7.8 Hz), 7.82 (d, 1H, J=8.3 Hz), 7.59 (t, 1H, J=7.6 Hz), 7.45 (t, 1H, J=7.5 Hz), 6.70-7.05 (m, 4H), 3.75-3.84 (m, 2H), 3.15-3.24 (m, 2H), 2.77 (d, 6H, J=5.0 Hz), 2.06-2.16 (m, 2H). ACPI-MS Found: [M+H]⁺=362.

### Example 4.42

2-(1-benzofuran-2-yl)-4-{[3-(dimethylamino)propyl]amino}-7-quinazolinecarbonitrile (42). A mixture of 2-(1-Benzofuran-2-yl)-4-oxo-3,4-dihydro-7-quinazolinecarboxamide (C: R=7-CONH₂, R′=1-benzofuran-2-yl) (0.199 g, 0.652 mmol) and tetramethylammonium chloride (0.14 g, 1.3 mmol) in POCl₃ (5 mL) was refluxed for 0.5 h to give the chloroquinazoline (H: R=7-CN, R′=benzofuran-2-yl). The chloroquinazoline was refluxed with N¹,N¹-dimethyl-1,3-propanediamine (0.25 mL, 2.0 mmol) in dioxane (20 mL) for 1 h, workup gave 42(0.186 g, 77%) as a solid. ¹H NMR (DMSO-d₆) δ ppm 8.76 (t, 1H, J=5.3 Hz), 8.39 (d, 1H, J=8.5 Hz), 8.29 (d, 1H, J=1.5 Hz), 7.87 (dd, 1H, J=8.4, 1.7 Hz), 7.79 (d, 1H, J=7.4 Hz), 7.70-7.77 (m, 2H), 7.43 (ddd, 1H, J=8.4, 7.3, 1.3 Hz), 7.32 (td, 1H, J=7.5, 0.9 Hz), 3.65-3.73 (m, 2H), 2.38 (t, 2H, J=6.9 Hz), 2.19 (s, 6H), 1.82-1.92 (m, 2H). ACPI-MS Found: [M+H]⁺=372.

### Example 4.43

2-(1-benzofuran-2-yl)-4-{[3-(dimethylamino)propyl]amino}-7-quinazolinecarboxamide dihydrochloride (43). A mixture of 2-(1-benzofuran-2-yl)-4-{[3-(dimethylamino)propyl]amino}-7-quinazolinecarbonitrile 42 (83 mg, 0.223 mmol) and KOH (0.166 g, 2.96 mmol) in t-butanol (5 mL) was refluxed in a sealed tube for 1 h. The mixture was quenched with brine (10 mL), extracted into EtOAc and washed with water. Removal of the solvent in vacuo gave an oil, conversion to the hydrochloride salt gave 43 (93 mg, 96%) as a solid. ¹H NMR (DMSO-d₆) δ ppm 10.26 (bs, 1H), 9.65 (bs, 1H), 8.53 (d, 1H, J=8.4 Hz), 8.45 (s, 1H), 8.29 (s, 1H), 8.11 (bs, 1H), 8.05 (dd, 1H, J=8.5, 1.1 Hz), 7.85 (d, 1H, J=7.7 Hz), 7.79 (d, 1H, J=8.2 Hz), 7.71 (bs, 1H), 7.52 (t, 1H, J=7.4 Hz), 7.40 (t, 1H, J=7.5 Hz), 3.81-3.88 (m, 2H), 3.21-3.28 (m, 2H), 2.78 (d, 6H, J=4.9 Hz), 2.13-2.22 (m, 2H). ACPI-MS Found: [M+H]⁺=390.

### Example 4.44

N¹-[2-(1-Benzofuran-2-yl)pyrido[2,3-d]pyrimidin-4-yl]-N³,N³-dimethyl-1,3-propanediamine dihydrochloride (44). A mixture of 2-(1-benzofuran-2-yl)pyrido[2,3-d]pyrimidin-4(3H)-one (C: R=8-aza, R′=benzofuran-2-yl) (0.136 g, 0.517 mmol) and tetramethylammonium chloride (0.113 g, 1.03 mmol) in POCl₃ (20 mL) was refluxed for 2 h to give the chloropyridopyrimidine (H: R=8-aza, R′=benzofuran-2-yl). The chloropyridopyrimidine was refluxed with N¹,N¹-dimethyl-1,3-propanediamine (0.18 mL, 1.43 mmol) in dioxane (20 mL) for 2 h, workup gave 44 (0.100 g, 56%) as a solid. ¹H NMR (DMSO-d₆) δ ppm 9.00 (dd, 1H, J=4.4, 1.8 Hz), 8.72 (t, 1H, J=5.3 Hz), 8.66 (dd, 1H, J=8.2, 1.8 Hz), 7.81 (d, 1H, J=7.5 Hz), 7.78 (d, 1H, J=7.5, 0.9 Hz), 7.71 (dd, 1H, J=8.2, 0.6 Hz), 7.52 (dd, 1H, J=8.2, 4.4 Hz), 7.44 (td, 1H, J=7.8, 1.3 Hz), 7.33 (td, 1H, J=7.5, 0.9 Hz), 3.71 (td, 2H, J=7.1, 5.3 Hz), 2.38 (t, 2H, J=6.9 Hz), 2.20 (s, 6H), 1.87 (ft, 2H, J=7.1, 6.9 Hz). ACPI-MS Found: [M+H]⁺=348.

### Example 4.45

N¹-[2-(1-Benzofuran-2-yl)-8-methyl-4-quinazolinyl]-N³,N³-dimethyl-1,3-propanediamine dihydrochloride (45). A mixture of 2-(1-benzofuran-2-yl)-8-methyl-4(3H)-quinazolinone (C: R=8-Me, R′=benzofuran-2-yl) (0.138 g, 0.499 mmol) and tetramethylammonium chloride (0.120 g, 1.09 mmol) in POCl₃ (5 mL) was refluxed for 30 min to give the chloroquinazoline (H: R=8-Me, R′=benzofuran-2-yl). The chloroquinazoline was refluxed with N¹,N¹-dimethyl-1,3-propanediamine (0.20 mL, 1.6 mmol) in dioxane (20 mL) for 2 h, workup and conversion to the hydrochloride salt gave 45 (0.133 g, 62%) as a solid. ¹H NMR (DMSO-d₆) δ ppm 10.32 (bs, 1H), 8.81 (bs, 1H), 8.18 (d, 1H, J=8.1 Hz), 7.86 (s, 1H), 7.79 (d, 1H, J=7.3 Hz), 7.75 (dd, 1H, J=8.3, 0.7 Hz), 7.70 (d, 1H, J=7.1 Hz), 7.41-7.48 (m, 2H), 7.34 (td, 1H, J=7.5, 0.9 Hz), 3.74-3.80 (m, 2H), 3.18-3.25 (m, 2H), 2.78 (d, 6H, J=5.0 Hz), 2.69 (s, 3H), 2.11-2.19 (m, 2H). ACPI-MS Found: [M+H]⁺=361.

### Example 4.46

N¹-[2-(1-Benzofuran-2-yl)-8-phenyl-4-quinazolinyl]-N³,N³-dimethyl-1,3-propanediamine dihydrochloride (46). A mixture of 2-(1-benzofuran-2-yl)-8-phenyl-4(3H)-quinazolinone (C: R=8-Ph, R′=benzofuran-2-yl) (0.199 g, 0.589 mmol) and tetramethylammonium chloride (0.130 g, 1.19 mmol) in POCl₃ (10 mL) was refluxed for 30 min to give the chloroquinazoline (H: R=8-Ph, R′=benzofuran-2-yl). The chloroquinazoline was refluxed with N¹,N¹-dimethyl-1,3-propanediamine (0.20 mL, 1.6 mmol) in dioxane (20 mL) for 2 h, workup and conversion to the hydrochloride salt gave 46 (0.098 g, 37%) as a solid. ¹H NMR (DMSO-d₆) δ ppm 9.99 (bs, 1H), 8.68 (bt, 1H, J=5.0 Hz), 8.31 (dd, 1H, J=8.3, 1.2 Hz), 7.86 (dd, 1H, J=7.3, 1.2 Hz), 7.74-7.81 (m, 3H), 7.59-7.68 (m, 3H), 7.50-7.56 (m, 2H), 7.37-7.47 (m, 2H), 7.31 (td, 1H, J=7.5, 0.8 Hz), 3.73-3.82 (m, 2H), 3.19-3.28 (m, 2H), 2.82 (d, 6H, J=5.0 Hz), 2.12-2.22 (m, 2H). ACPI-MS Found: [M+H]⁺=423.

### Example 4.47

N¹-[2-(1-Benzofuran-2-yl)-8-(trifluoromethyl)-4-quinazolinyl]-N³,N³-dimethyl-1,3-propanediamine dihydrochloride (47). A mixture of the crude 2-(1-benzofuran-2-yl)-8-(trifluoromethyl)-4(3H)-quinazolinone (C: R=8-CF₃, R=benzofuran-2-yl) (used directly from the quinazolinone formation) and tetramethylammonium chloride (0.310 g, 2.83 mmol) in POCl₃ (10 mL) was refluxed for 1 h to give the chloroquinazoline (H: R=8-CF₃, R′=benzofuran-2-yl). The chloroquinazoline was refluxed with N¹,N¹-dimethyl-1,3-propanediamine (0.50 mL, 4.0 mmol) in dioxane (20 mL) for 2 h, workup and conversion to the hydrochloride salt gave 47 (0.072 g, 10%) as a solid. ¹H NMR (DMSO-d₆) δ ppm 9.81 (bs, 1H), 8.89 (t, 1H, J=5.5 Hz), 8.57 (d, 1H, J=7.8 Hz), 8.20 (d, 1H, J=7.3 Hz), 7.78-7.82 (m, 2H), 7.75 (dd, 1H, J=8.3, 0.7 Hz), 7.66 (t, 1H, J=7.8 Hz), 7.45 (ddd, 1H, J=8.3, 7.3, 1.3 Hz), 7.34 (td, 1H, J=7.5, 0.9 Hz), 3.73-3.80 (m, 2H), 3.19-3.26 (m, 2H), 2.81 (d, 6H, J=5.0 Hz), 2.09-2.19 (m, 2H). ACPI-MS Found: [M+H]⁺=415.

### Example 4.48

N¹-[2(1-Benzofuran-2-yl)-8-methoxy-4-quinazolinyl]-N³,N³-dimethyl-1,3-propanediamine dihydrochloride (48). A mixture of 2-(1-benzofuran-2-yl)-8-methoxy-4(3H)-quinazolinone (C: R=8-OMe, R′=benzofuran-2-yl) (0.148 g, 0.506 mmol) in thionyl chloride (10 mL)/dmf (0.1 mL) was refluxed for 10 min to give the chloroquinazoline (H: R=8-OMe, R′=benzofuran-2-yl). The chloroquinazoline was refluxed with N¹,N¹-dimethyl-1,3-propanediamine (0.20 mL, 1.6 mmol) in dioxane (40 mL) for 2 h, workup and conversion to the hydrochloride salt gave 48 (0.109 g, 48%) as a solid. ¹H NMR (DMSO-d₅) δ ppm 10.37 (bs, 1H), 9.25 (bs, 1H), 7.92-7.99 (m, 2H), 7.81 (d, 1H, J=5.8 Hz), 7.73 (dd, 1H, J=8.4, 0.5 Hz), 7.55 (t, 1H, J=8.1 Hz), 7.49 (td, 1H, J=7.8, 1.2 Hz), 7.43 (d, 1H, J=7.8 Hz), 7.37 (td, 1H, J=7.5, 0.8 Hz), 4.02 (s, 3H), 3.76-3.83 (m, 2H), 3.15-3.24 (m, 2H), 2.77 (d, 6H, J=5.0 Hz), 2.13-2.20 (m, 2H). ACPI-MS Found: [M+H]⁺=377.

### Example 4.49

N¹-[2-(1-Benzofuran-2-yl)-8-chloro-4-quinazolinyl]-N³,N³-dimethyl-1,3-propanediamine dihydrochloride (49). A mixture of 2-(1-benzofuran-2-yl)-8-chloro-4(3H)-quinazolinone (C: R=8-Cl, R′=benzofuran-2-yl) (0.042 g, 0.142 mmol) and tetramethylammonium chloride (0.031 g, 0.28 mmol) in POCl₃ (5 mL) was refluxed for 0.5 h to give the chloroquinazoline (H: R=8-Cl, R′=benzofuran-2-yl). The chloroquinazoline was refluxed with N¹,N¹-dimethyl-1,3-propanediamine (0.05 mL, 0.4 mmol) in dioxane (10 mL) for 2 h, workup and conversion to the hydrochloride salt gave 49 (0.051 g, 79%) as a solid. ¹H NMR (DMSO-d₆) δ ppm 9.82 (bs, 1H), 8.76 (t, 1H, J=5.8 Hz), 8.26 (dd, 1H, J=8.4, 1.1 Hz), 7.99 (dd, 1H, J=7.6, 1.0 Hz), 7.84 (d, 1H, J=0.8 Hz), 7.80 (d, 1H, J=7.3 Hz), 7.76 (dd, 1H, J=8.4, 0.6 Hz), 7.51 (t, 1H, J=8.0 Hz), 7.45 (ddd, 1H, J=7.5, 0.8 Hz), 7.34 (td, 1H, J=7.5, 0.8 Hz), 3.71-3.78 (m, 2H), 3.18-3.26 (m, 2H), 2.80 (d, 6H, J=5.0 Hz), 2.08-2.18 (m, 2H). ACPI-MS Found: [M+H]⁺=381, 383.

### Example 4.50

N¹-[2-(1-Benzofuran-2-yl)-8-nitro-4-quinazolinyl]-N³,N³-dimethyl-1,3-propanediamine (50). A mixture of 2-(1-benzofuran-2-yl)-8-nitro-4(3H)-quinazolinone (C: R=8-NO₂, R′=benzofuran-2-yl) (0.400 g, 1.30 mmol) and tetramethylammonium chloride (0.290 g, 2.65 mmol) in POCl₃ (20 mL) was refluxed for 1 h to give the chloroquinazoline (H: R=8-NO₂, R′=benzofuran-2-yl). The chloroquinazoline was refluxed with N¹,N¹-dimethyl-1,3-propanediamine (0.45 mL, 3.58 mmol) in dioxane (40 mL) for 2 h, workup gave 50 (0.110 g, 22%) as a solid. ¹H NMR (DMSO-d₆) δ ppm 8.87 (t, 1H, J=5.3 Hz), 8.48 (dd, 1H, J=8.4, 1.2 Hz), 8.25 (dd, 1H, J=7.6, 1.2 Hz), 7.71-7.82 (m, 3H), 7.64 (t, 1H, J=8.1 Hz), 7.44 (ddd, 1H, J=8.2, 7.2, 1.3 Hz), 7.33 (td, 1H, J=7.5, 0.9 Hz), 3.67-3.74 (m, 2H), 2.39 (t, 2H, J=6.9 Hz), 2.20 (s, 6H), 1.84-1.93 (m, 2H). ACPI-MS Found: [M+H]⁺=392.

### Example 4.51

N¹-[2-(1-Benzofuran-2-yl)-8-amino-4-quinazolinyl]-N³,N³-dimethyl-1,3-propanediamine dihydrochloride (51). A solution of N¹-[2-(1-benzofuran-2-yl)-8-nitro-4-quinazolinyl]-N³,N³-dimethyl-1,3-propanediamine 50 (0.079 g, 0.202 mmol) and 5% Pd on carbon (20 mg) in methanol (40 mL) was hydrogenated (40 p.s.i.) for 22 h. The solution was filtered and the solvent removed in vacuo, conversion to the hydrochloride salt gave 51 (63 mg, 90%) as a solid. ¹H NMR (DMSO-d₆) δ ppm 10.16 (bs, 1H), 8.63 (bs, 1H), 7.91 (s, 1H), 7.78 (d, 1H, J=7.4 Hz), 7.73 (dd, 1H, J=8.3, 0.6 Hz), 7.61 (bd, 1H, J=7.5 Hz), 7.44 (ddd, 1H, J=8.3, 7.3, 1.3 Hz), 7.30-7.37 (m, 2H), 7.18 (bd, 1H, J=7.3 Hz), 3.72-3.80 (m, 2H), 3.17-3.25 (m, 2H), 2.79 (d, 6H, J=5.0 Hz), 2.08-2.17 (m, 2H). ACPI-MS Found: [M+H]⁺=362.

### Example 4.52

2-(1-benzofuran-2-yl)-4-{[3-(dimethylamino)propyl]amino}-8-quinazolinecarbonitrile (52). A mixture of 2-(1-Benzofuran-2-yl)-4-oxo-3,4-dihydro-8-quinazolinecarboxamide (C: R=8-CONH₂, R′=benzofuran-2-yl) (0.325 g, 1.07 mmol) and tetramethylammonium chloride (0.24 g, 2.2 mmol) in POCl₃ (10 mL) was refluxed for 0.5 h to give the chloroquinazoline (H: R=8-CN, R′=benzofuran-2-yl). The chloroquinazoline was refluxed with N¹,N¹-dimethyl-1,3-propanediamine (0.40 mL, 3.2 mmol) in dioxane (30 mL) for 1 h, workup gave 52 (0.211 g, 53%) as a solid. ¹H NMR (DMSO-d₆) δ ppm 8.83 (t, 1H, J=5.1 Hz), 8.52 (dd, 1H, J=8.3, 1.3 Hz), 8.32 (dd, 1H, J=7.4, 1.2 Hz), 7.79-7.84 (m, 2H), 7.76 (dd, 1H, J=8.3, 0.7 Hz), 7.63 (td, 1H, J=7.8, 0.6 Hz), 7.45 (ddd, 1H, J=8.4, 7.2, 1.3 Hz), 7.34 (td, 1H, J=7.5, 0.9 Hz), 3.67-3.74 (m, 2H), 2.38 (t, 2H, J=6.9 Hz), 2.20 (s, 6H), 1.84-1.92 (m, 2H). ACPI-MS Found: [M+H]⁺=372.

### Example 4.53

2-(1-benzofuran-2-yl)-4-{[3-(dimethylamino)propyl]amino}-8-quinazolinecarboxamide (53). A mixture of 2-(1-benzofuran-2-yl)-4-{[3-(dimethylamino)propyl]amino}8-quinazolinecarbonitrile (52) (0.125 g, 0.337 mmol) and KOH (0.250 g, 4.46 mmol) in t-butanol (10 mL) was refluxed in a sealed tube for 1 h. The mixture was quenched with brine (10 mL), extracted into EtOAc and washed with water. Removal of the solvent in vacuo gave 53 (0.099 g, 76%) as a solid. ¹H NMR (DMSO-d₆) δ ppm 10.67 (d, 1H, J=4.1 Hz), 8.61 (t, 1H, J=5.2 Hz), 8.59 (dd, 1H, J=7.5, 1.4 Hz), 8.42 (dd, 1H, J=8.2, 1.4 Hz), 7.87-7.91 (m, 1H), 7.81 (d, 1H, J=7.3 Hz), 7.78 (d, 1H, J=0.9 Hz), 7.59-7.67 (m, 2H), 7.46 (ddd, 1H, J=8.4, 7.3, 1.3 Hz), 7.34 (td, 1H, J=7.5, 0.9 Hz), 3.67-3.74 (m, 2H), 2.41 (t, 2H, J=6.9 Hz), 2.22 (s, 6H), 1.84-1.92 (m, 2H). ACPI-MS Found: [M+H]⁺=390.

### Example 4.54

N¹-[2-(1-Benzofuran-2-yl)benzo[g]quinazolin-4-yl]-N³,N³-dimethyl-1,3-propanediamine dihydrochloride (54). A mixture of 2-(1-benzofuran-2-yl)benzo[g]quinazolin-4(3H)-one (C: R=6,7-benz, R′=benzofuran-2-yl) (0.435 g, 1.40 mmol) and tetramethylammonium chloride (0.308 g, 2.81 mmol) in POCl₃ (10 mL) was refluxed for 0.5 h to give the chloroquinazoline (H: R=6,7-benz, R′=benzofuran-2-yl). The chloroquinazoline was refluxed with N¹,N¹-dimethyl-1,3-propanediamine (0.53 mL, 4.21 mmol) in dioxane (40 mL) for 2 h, workup and conversion to the hydrochloride salt gave 54 (0.058 g, 9%) as a solid. ¹H NMR (DMSO-d₆) δ ppm 10.37 (bs, 1H), 9.35 (bs, 1H), 8.57 (s, 1H), 8.33 (bs, 1H), 8.19 (d, 1H, J=8.3 Hz), 8.12 (d, 1H, J=8.3 Hz), 7.91 (d, 1H, J=7.7 Hz), 7.85 (d, 1H, J=8.3 Hz), 7.78 (t, 1H, J=7.4 Hz), 7.70 (t, 1H, J=7.5 Hz), 7.60 (t, 1H, J=7.7 Hz), 7.45 (t, 1H, J=7.5 Hz), 3.94-4.00 (m, 2H), 3.24-3.32 (m, 2H), 2.78 (d, 6H, J=5.0 Hz), 2.20-2.29 (m, 2H). ACPI-MS Found: [M+H]⁺=397.

### Example 4.55

N¹-[2-(1-Benzofuran-2-yl)-6,7-dichloro-4-quinazolinyl]-N³,N³-dimethyl-1,3-propanediamine dihydrochloride (55). A mixture of 2-(1-benzofuran-2-yl)-6,7-dichloro-4(3H)-quinazolinone (C: R=6,7-diCl, R′=benzofuran-2-yl) (0.104 g, 0.314 mmol) and tetramethylammonium chloride (0.070 g, 0.64 mmol) in POCl₃ (10 mL) was refluxed for 0.5 h to give the chloroquinazoline (H: R=6,7-diCl, R′=benzofuran-2-yl). The chloroquinazoline was refluxed with N¹,N¹-dimethyl-1,3-propanediamine (0.11 mL, 0.874 mmol) in dioxane (20 mL) for 2 h, workup and conversion to the hydrochloride salt gave 55 (0.080 g, 52%) as a solid. ¹H NMR (DMSO-d₆) δ ppm 10.03 (bs, 1H), 9.07 (bs, 1H), 8.74 (s, 1H), 8.12 (s, 1H), 7.80 (d, 1H, J=7.4 Hz), 7.75 (dd, 1H, J=8.3, 0.6 Hz), 7.47 (ddd, 1H, J=8.3, 7.3, 1.3 Hz), 7.35 (td, 1H, J=7.5, 0.8 Hz), 3.73-3.80 (m, 2H), 3.20-3.26 (m, 2H), 2.78 (d, 6H, J=5.0 Hz), 2.08-2.18 (m, 2H). ACPI-MS Found: [M+H]⁺=415, 417, 419.

### Example 4.56

N¹-[2-(1-Benzofuran-2-yl)-6,8-dichloro-4-quinazolinyl]-N³,N³-dimethyl-1,3-propanediamine dihydrochloride (56). A mixture of 2-(1-benzofuran-2-yl)-6,8-dichloro-4(3H)-quinazolinone (C: R=6,8-diCl, R′=benzofuran-2-yl) (0.613 g, 1.85 mmol) and tetramethylammonium chloride (0.410 g, 3.74 mmol) in POCl₃ (20 mL) was refluxed for 1 h to give the chloroquinazoline (H: R=6,8-diCl, R′=benzofuran-2-yl). The chloroquinazoline was refluxed with N¹,N¹-dimethyl-1,3-propanediamine (0.65 mL, 5.17 mmol) in dioxane (40 mL) for 2 h, workup and conversion to the hydrochloride salt gave 56 (0.710 g, 92%) as a solid. ¹H NMR (DMSO-d₆) δ ppm 10.03 (bs, 1H), 8.64 (bt, 1H, J=4.8 Hz), 8.39 (d, 1H, J=2.2 Hz), 8.08 (d, 1H, J=2.2 Hz), 7.80 (d, 1H, J=7.4 Hz), 7.77 (d, 1H, J=0.9 Hz), 7.73 (dd, 1H, J=8.3, 0.7 Hz), 7.44 (ddd, 1H, J=8.3, 7.4, 1.3 Hz), 7.32 (td, 1H, J=7.4, 0.8 Hz), 3.68-3.72 (m, 2H), 2.37 (t, 2H, J=7.0 Hz), 2.19 (s, 6H), 1.82-1.90 (m, 2H). ACPI-MS Found: [M+H]⁺=415, 417, 419.

### Example 4.57

N¹-[2-(1-Benzofuran-2-yl)-6,8-dibromo-4-quinazolinyl]-N³,N³-dimethyl-1,3-propanediamine (57). A mixture of 2-(1-benzofuran-2-yl)-6,8-dibromo-4(3H)-quinazolinone (C: R=6,8-diBr, R′=benzofuran-2-yl) (0.187 g, 0.445 mmol) and tetramethylammonium chloride (0.100 g, 0.912 mmol) in POCl₃ (20 mL) was refluxed for 0.5 h to give the chloroquinazoline (H: R=6,8-diBr, R′=benzofuran-2-yl). The chloroquinazoline was refluxed with N¹,N¹-dimethyl-1,3-propanediamine (0.15 mL, 1.19 mmol) in dioxane (20 mL) for 2 h, workup gave 57 (0.139 g, 58%) as a solid. ¹H NMR (DMSO-d₆) δ ppm 8.66 (bt, 1H, J=5.2 Hz), 8.57 (d, 1H, J=2.0 Hz), 8.32 (d, 1H, J=2.0 Hz), 7.80 (d, 1H, J=7.3 Hz), 7.77 (d, 1H, J=0.9 Hz), 7.73 (dd, 1H, J=8.3, 0.7 Hz), 7.44 (ddd, 1 h, J=8.3, 7.3, 1.3 Hz), 7.33 (td, 1H, J=7.5, 0.9 Hz), 3.65-3.72 (m, 2H), 2.37 (t, 2H, J=7.0 Hz), 2.19 (s, 6H), 1.82-1.91 (m, 2H). ACPI-MS Found: [M+H]⁺=503, 505, 507.

### Example 4.58

N¹-[2-(1-Benzofuran-2-yl)-7,8-dimethyl-4-quinazolinyl]-3,N-dimethyl-1,3-propanediamine dihydrochloride (58). A mixture of 2-(1-benzofuran-2-yl)-7,8-dimethyl-4(3H)-quinazolinone (C: R=7,8-diMe, R′=benzofuran-2-yl) (0.223 g, 0.768 mmol) and tetramethylammonium chloride (0.160 g, 1.46 mmol) in POCl₃ (10 mL) was refluxed for 15 min to give the chloroquinazoline (H: R=7,8-diMe, R′=benzofuran-2-yl). The chloroquinazoline was refluxed with N¹,N¹-dimethyl-1,3-propanediamine (0.30 mL, 2.38 mmol) in dioxane (40 mL) for 2 h, workup and conversion to the hydrochloride salt gave 58 (0.313 g, 91%) as a solid. ¹H NMR (DMSO-d₆) δ ppm 10.20 (s, 1H), 8.65 (s, 1H), 8.07 (d, 1H, J=8.4 Hz), 7.83 (s, 1H), 7.78 (d, 1H, J=7.4 Hz), 7.75 (dd, 1H, J=8.4, 0.6 Hz), 7.43 (td, 1H, J=7.7, 1.3 Hz), 7.39 (d, 1H, J=8.4 Hz), 7.34 (td, 1H, J=7.5 Hz), 3.71-3.78 (m, 2H), 3.10-3.23 (m, 2H), 2.78 (d, 6H, 5.0 Hz), 2.64 (s, 3H), 2.45 (s, 3H), 2.09-2.20 (m, 2H). ACPI-MS Found: [M+H]⁺=375.

### Example 4.59

N¹-[2-(1-Benzofuran-2-yl)-7,8-dimethoxy-4-quinazolinyl]-N³,N³-dimethyl-1,3-propanediamine dihydrochloride (59). A mixture of the crude 2-(1-benzofuran-2-yl)-7,8-dimethoxy-4(3H)-quinazolinone (C: R=7,8-diOMe, R′=benzofuran-2-yl) and tetramethylammonium chloride (0.150 g, 1.37 mmol) in POCl₃ (10 mL) was refluxed for 2 h to give the chloroquinazoline (H: R=7,8-diOMe, R′=benzofuran-2-yl). The chloroquinazoline was refluxed with N¹,N¹-dimethyl-1,3-propanediamine (0.21 mL, 1.33 mmol) in dioxane (20 mL) for 2 h, workup and conversion to the hydrochloride salt gave 59 (0.019 g, 6%) as a solid. ¹H NMR (DMSO-d₆) δ ppm 10.09 (bs, 1H), 9.03 (bs, 1H), 8.14 (d, 1H, J=9.0 Hz), 7.94 (s, 1H), 7.82 (d, 1H, J=7.0 Hz), 7.77 (d, 1H, J=8.2 Hz), 7.42-7.50 (m, 2H), 7.36 (t, 1H, J=7.5 Hz), 4.01 (s, 3H), 3.99 (s, 3H), 3.72-3.80 (m, 2H), 3.19-3.26 (m, 2H), 2.78 (d, 6H, J=5.0 Hz), 2.08-2.17 (m, 2H). ACPI-MS Found: [M+H]⁺=407.

### Example 4.60

N¹,N¹-Dimethyl-N³-[2-(3-methyl-1-benzofuran-2-yl)-4-quinazolinyl]-1,3-propanediamine dihydrochloride (60). A mixture of 2-(3-methyl-1-benzofuran-2-yl)-4(3H)-quinazolinone (C: R═H, R′=3-methyl-1-benzofuran-2-yl) (0.707 g, 2.56 mmol) in SOCl₂ (6 mL)/dmf (0.1 mL) was refluxed for 10 min. to give the chloroquinazoline (H: R═H, R′=3-methyl-1-benzofuran-2-yl). The chloroquinazoline was refluxed with N¹,N¹-dimethyl-1,3-propanediamine (0.65 mL, 5.17 mmol) in dioxane (60 mL) for 2 h, workup and conversion to the hydrochloride salt gave 60 (0.238 g, 21%) as a solid. ¹H NMR (DMSO-d₆) δ ppm 14.2 (bs, 1H), 10.66 (s, 1H), 10.57 (s, 1H), 8.71 (d, 1H, J=8.2 Hz), 8.11 (d, 1H, J=8.1 Hz), 8.02 (td, 1H, J=7.7, 0.9 Hz), 7.93 (d, 1H, J=7.8 Hz), 7.78 (d, 1H, J=8.4 Hz), 7.74 (td, 1H, J=7.7, 0.9 Hz), 7.61 (td, 1H, J=7.7, 1.1 Hz), 7.46 (td, 1H, J=7.5, 0.7 Hz), 3.81-3.90 (m, 2H), 3.17-3.26 (m, 2H), 2.85 (s, 3H), 2.76 (d, 6H, J=4.8 Hz), 2.19-2.27 (m, 2H). ACPI-MS Found: [M+H]⁺=361.

### Examples 4.61 and 4.62

N¹-[2-(4-chloro-5-methoxy-1-benzofuran-2-yl)-4-quinazolinyl]-N¹,N¹-dimethyl-1,3-propanediamine hydrochloride (61) and N¹-[2-(5-methoxy-1-benzofuran-2-yl)-4-quinazolinyl]-N¹,N¹-dimethyl-1,3-propanediamine (62). A mixture of 2-(5-methoxy-1-benzofuran-2-yl)-4(3H)-quinazolinone (C: R═H, R′=5-methoxy-1-benzofuran-2-yl) (0.111 g, 0.380 mmol) in SOCl₂ (12 mL)/dmf (0.1 mL) was refluxed for 1.5 h to give the chloroquinazoline (H: R═H, R=5-methoxy-1-benzofuran-2-yl). The chloroquinazoline was refluxed with N¹,N¹-dimethyl-1,3-propanediamine (0.50 mL, 4.0 mmol) in dioxane (10 mL) for 2 h, workup, HPLC and conversion to the hydrochloride salt gave N¹-[2-(4-chloro-5-methoxy-1-benzofuran-2-yl)-4-quinazolinyl]-N¹,N¹-dimethyl-1,3-propanediamine (61) (0.026 g, 14%). ¹H NMR (DMSO-d₆) δ ppm 8.52 (t, 1H, J=5.4 Hz), 8.22 (d, 1H, J=8.2 Hz), 7.77-7.83 (m, 2H), 7.69 (dd, 1H, J=9.0, 0.9 Hz), 7.59 (d, 1H, J=0.9 Hz), 7.50-7.56 (m, 1H), 7.28 (d, 1H, J=9.1 Hz), 4.11 (s, 3H), 3.65-3.72 (m, 2H), 2.39 (t, 2H, J=6.9 Hz), 2.22 (s, 6H), 1.82-1.91 (m, 2H). ACPI-MS Found: [M+H]⁺=411, 413; and N¹-[2-(5-methoxy-1-benzofuran-2-yl)-4-quinazolinyl]-N¹,N¹-dimethyl-1,3-propanediamine (62) (14 mg, 8%). ¹H NMR (DMSO-d₆) δ ppm 8.70 (bs, 1H), 7.98 (dd, 1H, J=8.3, 0.5 Hz), 7.71 (ddd, 1H, J=8.4, 7.0, 1.3 Hz), 7.65 (d, 1H, J=0.9 Hz), 7.62 (dd, 1H, J=7.8, 0.7 Hz), 7.56 (d, 1H, J=9.0 Hz), 7.42 (ddd, 1H, J=8.1, 7.0, 1.1 Hz), 7.10 (d, 1H, J=2.5 Hz), 6.96 (dd, 1H, J=9.0, 2.5 Hz), 3.86 (s, 3H), 3.86-3.91 (m, 2H), 2.62-2.66 (m, 2H), 2.41 (s, 6H), 1.87-1.96 (m, 2H). ACPI-MS Found: [M+H]⁺=377. The compounds were separated by preparative HPLC.

### Example 4.63

N¹,N¹-dimethyl-N³-[2-(5-methyl-1-benzofuran-2-yl)-4-quinazolinyl]-1,3-propanediamine dihydrochloride (63). A mixture of 2-(5-methyl-1-benzofuran-2-yl)-4(3H)-quinazolinone (C: R═H, R′=5-methyl-1-benzofuran-2-yl) (0.330 g, 1.19 mmol) and tetramethylammonium chloride (0.26 g, 2.4 mmol) in POCl₃ (10 mL) was refluxed for 0.5 h to give the chloroquinazoline (H: R═H, R′=5-methyl-1-benzofuran-2-yl). The chloroquinazoline was refluxed with N¹,N¹-dimethyl-1,3-propanediamine (0.45 mL, 3.6 mmol) in dioxane (30 mL) for 2 h, workup gave 63 (0.490 g, 95%) as a solid. ¹H NMR (DMSO-d₆) δ ppm 10.45 (bs, 1H), 10.16 (bs, 1H), 8.58 (d, 1H, J=8.0 Hz), 8.22 (s, 1H), 8.08 (d, 1H, J=8.3 Hz), 7.99 (t, 1H, J=7.4 Hz), 7.61-7.75 (m, 3H), 7.39 (d, 1H, J=8.7 Hz), 3.82-3.91 (m, 2H), 3.19-3.28 (m, 2H), 2.77 (d, 6H, J=4.9 Hz), 2.46 (s, 3H), 2.13-2.23 (m, 2H). ACPI-MS Found: [M+H]⁺=361.

### Example 4.64

N¹,N¹-dimethyl-N³-[2-(5-chloro-1-benzofuran-2-yl)-4-quinazolinyl]-1,3-propanediamine (64). A mixture of 2-(5-chloro-1-benzofuran-2-yl)-4(3H)-quinazolinone (C: R═H, R′=5-chloro-1-benzofuran-2-yl) (0.130 g, 0.438 mmol) and tetramethylammonium chloride (0.10 g, 2.34 mmol) in POCl₃ (10 mL) was refluxed for 0.5 h to give the chloroquinazoline (H: R═H, R′=5-chloro-1-benzofuran-2-yl). The chloroquinazoline was refluxed with N¹,N¹-dimethyl-1,3-propanediamine (0.165 mL, 1.31 mmol) in dioxane (20 mL) for 2 h, workup gave 64 (0.127 g, 76%) as a solid. ¹H NMR (DMSO-d₆) δ ppm 8.50 (t, 1H, J=5.2 Hz), 8.21 (d, 1H, J=8.2 Hz), 7.86 (d, 1H, J=2.2 Hz), 7.78-7.82 (m, 2H), 7.76 (d, 1H, J=8.8 Hz), 7.67 (s, 1H), 7.50-7.58 (m, 1H), 7.43 (dd, 1H, J=8.8, 2.2 Hz), 3.64-3.71 (m, 2H), 2.38 (t, 2H, J=6.9 Hz), 2.20 (s, 6H), 1.82-1.91 (m, 2H). ACPI-MS Found: [M+H]⁺=383, 381.

### Example 4.65

N¹-[2-(5-Bromo-1-benzofuran-2-yl)-4-quinazolinyl]-N³,N³-dimethyl-1,3-propanediamine (65). A mixture of 2-(5-bromo-1-benzofuran-2-yl)-4(3H)-quinazolinone (C: R═H, R′=5-bromo-1-benzofuran-2-yl) (0.333 g, 0.976 mmol) and tetramethylammonium chloride (0.22 g, 2.01 mmol) in POCl₃ (15 mL) was refluxed for 0.5 h to give the chloroquinazoline (H: R═H, R′=5-bromo-1-benzofuran-2-yl). The chloroquinazoline was refluxed with N¹,N¹-dimethyl-1,3-propanediamine (0.37 mL, 2.94 mmol) in dioxane (30 mL) for 2 h, workup gave 65 (0.337 g, 80%) as a solid. ¹H NMR (DMSO-d₆) δ ppm 8.50 (t, 1H, J=5.1 Hz), 8.22 (d, 1H, J=8.2 Hz), 8.01 (d, 1H, J=2.0 Hz), 7.78-7.82 (m, 2H), 7.71 (d, 1H, J=8.8 Hz), 7.66 (d, 1H, J=0.9 Hz), 7.51-7.57 (m, 2H), 3.65-3.72 (m, 2H), 2.37 (t, 2H, J=6.9 Hz), 2.20 (s, 6H), 1.82-1.91 (m, 2H). ACPI-MS Found: [M+H]⁺=427, 425.

### Example 4.66

N¹-[2-(6-Methoxy-1-benzofuran-2-yl)-4-quinazolinyl]-N¹,N¹-dimethyl-1,3-propanediamine dihydrochloride (66). A mixture of 2-(6-methoxy-1-benzofuran-2-yl)-4(3H)-quinazolinone (C: R═H, R′=6-methoxy-1-benzofuran-2-yl) (0.406 g, 1.38 mmol) in SOCl₂ (5 mL)/dmf (0.1 mL) was refluxed for 1 h to give the chloroquinazoline (H: R═H, R′=6-methoxy-1-benzofuran-2-yl). The chloroquinazoline was refluxed with N¹,N¹-dimethyl-1,3-propanediamine (0.52 mL, 4.13 mmol) in dioxane (25 mL) for 2 h, workup and conversion to the hydrochloride salt gave 66 (0.479 g, 77%) as a solid. ¹H NMR (DMSO-d₆) δ ppm 14.0 (b, 1H), 10.59 (bs, 1H), 10.33 (bs, 1H), 8.62 (d, 1H, J=8.2 Hz), 8.33 (s, 1H), 8.10 (d, 1H, J=8.4 Hz), 7.99 (t, 1H, J=7.5 Hz), 7.78 (d, 1H, J=8.7 Hz), 7.71 (t, 1H, J=7.7 Hz), 7.32 (d, 1H, J=0.6 Hz), 7.07 (dd, 1H, J=8.7, 2.0 Hz), 3.90 (s, 3H), 3.82-3.90 (m, 2H), 3.18-3.26 (m, 2H), 2.76 (d, 6H, J=5.0 Hz), 2.16-2.23 (m, 2H). ACPI-MS Found: [M+H]⁺=377.

### Example 4.67

N¹,N¹-dimethyl-N³-[2-(7-methyl-1-benzofuran-2-yl)-4-quinazolinyl]-1,3-propanediamine dihydrochloride (67). A mixture of 2-(7-methyl-1-benzofuran-2-yl)-4(3H)-quinazolinone (C: R═H, R′=7-methyl-1-benzofuran-2-yl) (0.108 g, 0.391 mmol) and tetramethylammonium chloride (0.090 g, 0.82 mmol) in POCl₃ (5 mL) was refluxed for 0.5 h to give the chloroquinazoline (H: R═H, R′=7-methyl-1-benzofuran-2-yl). The chloroquinazoline was refluxed with N¹,N¹-dimethyl-1,3-propanediamine (0.15 mL, 1.2 mmol) in dioxane (10 mL) for 2 h, workup and conversion to the hydrochloride salt gave 67 (0.166 g, 98%) as a solid. ¹H NMR (DMSO-d₆) δ ppm 10.43 (bs, 1H), 10.13 (bs, 1H), 8.58 (d, 1H, J=8.1 Hz), 8.28 (s, 1H), 8.16 (d, 1H, J=8.2 Hz), 8.00 (t, 1H, J=7.6 Hz), 7.65-7.76 (m, 2H), 7.38 (d, 1H, J=7.2 Hz), 7.32 (t, 1H, J=7.6 Hz), 3.85-3.93 (m, 2H), 3.19-3.27 (m, 2H), 2.77 (d, 6H, J=4.9 Hz), 2.62 (s, 3H), 2.15-2.24 (m, 2H). ACPI-MS Found: [M+H]⁺=361.

### Example 4.68

N¹,N¹-dimethyl-N³-[2-(7-methoxy-1-benzofuran-2-yl)-4-quinazolinyl]-1,3-propanediamine dihydrochloride (68). A mixture of 2-(7-methoxy-1-benzofuran-2-yl)-4(3H)-quinazolinone (C: R═H, R′=7-methoxy-1-benzofuran-2-yl) (0.342 g, 1.17 mmol) and tetramethylammonium chloride (0.256 g, 2.34 mmol) in POCl₃ (10 mL) was refluxed for 0.5 h to give the chloroquinazoline (H: R═H, R′=7-methoxy-1-benzofuran-2-yl). The chloroquinazoline was refluxed with N¹,N¹-dimethyl-1,3-propanediamine (0.44 mL, 3.5 mmol) in dioxane (40 mL) for 2 h, workup and conversion to the hydrochloride salt gave 68 (0.436 g, 83%) as a solid. ¹H NMR (DMSO-d₆) δ ppm 10.44 (bs, 1H), 10.06 (bs, 1H), 8.57 (d, 1H, J=8.2 Hz), 8.26 (s, 1H), 8.09 (d, 1H, J=8.3 Hz), 7.98 (t, 1H, J=7.3 Hz), 7.71 (t, 1H, J=7.5 Hz), 7.42 (dd, 1H, J=7.9, 0.7 Hz), 7.34 (t, 1H, J=7.9 Hz), 7.18 (d, 1H, J=7.6 Hz), 4.03 (s, 3H), 3.83-3.90 (m, 2H), 3.18-3.27 (m, 2H), 2.77 (d, 6H, J=4.9 Hz), 2.15-2.23 (m, 2H). ACPI-MS Found: [M+H]⁺=377.

### Example 4.69

N¹,N¹-Dimethyl-N³-[8-methyl-2-(3-methyl-1-benzofuran-2-yl)-4-quinazolinyl]-1,3-propanediamine dihydrochloride (69). A mixture of 8-methyl-2-(3-methyl-1-benzofuran-2-yl)-4(3H)-quinazolinone (C: R=8-Me, R′=3-methyl-1-benzofuran-2-yl) (0.489 g, 1.68 mmol) and tetramethylammonium chloride (0.370 g, 3.38 mmol) in POCl₃ (10 mL) was refluxed for 0.5 h to give the chloroquinazoline (H: R=8-Me, R′=3-methyl-1-benzofuran-2-yl). The chloroquinazoline was refluxed with N¹,N¹-dimethyl-1,3-propanediamine (0.73 mL, 4.62 mmol) in dioxane (60 mL) for 2 h, workup and conversion to the hydrochloride salt gave 69 (0.547 g, 76%) as a solid. ¹H NMR (DMSO-d₆) δ ppm 10.40 (bs, 1H), 9.10 (bs, 1H), 8.25 (d, 1H, J=8.0 Hz), 7.81 (d, 1H, J=7.7 Hz), 7.69-7.76 (m, 2H), 7.44-7.51 (m, 2H), 7.37 (td, 1H, J=7.4, 0.5 Hz), 3.75-3.85 (m, 2H), 3.17-3.26 (m, 2H), 2.82 (s, 3H), 2.78 (d, 6H, J=5.0 Hz), 2.69 (s, 3H), 2.12-2.21 (m, 2H). ACPI-MS Found: [M+H]⁺=375.

### Example 4.70

N¹-[2-(5-Methoxy-1H-indol-2-yl)-4-quinazolinyl]-N³,N³-dimethyl-1,3-propanediamine dihydrochloride (70). A mixture of 2-(5-methoxy-1H-indol-2-yl)-4(3H)-quinazolinone (C: R=5-H, R′=5-methoxy-1H-indol-2-yl) (0.956 g, 3.28 mmol) and tetramethylammonium chloride (0.72 g, 6.57 mmol) in POCl₃ (20 mL) was refluxed for 15 min to give the chloroquinazoline (H: R═H, R′=5-methoxy-1H-indol-2-yl) (0.592 g, 58%). The chloroquinazoline (0.517 g, 1.67 mmol) was refluxed with N¹,N¹-dimethyl-1,3-propanediamine (0.63 mL, 5.01 mmol) in dioxane (50 mL) for 2 h, workup and conversion to the hydrochloride salt gave 70 (0.586 g, 78%) as a solid. ¹H NMR (DMSO-d₆) δ ppm 14.55 (bs, 1H), 11.96 (bs, 1H), 10.53 (bs, 1H), 10.37 (bs, 1H), 8.56 (d, 1H, J=8.0 Hz), 8.16 (bd, 1H, J=6.8 Hz), 8.00 (t, 1H, J=7.5 Hz), 7.69 (t, 1H, J=7.6 Hz), 7.61 (d, 1H, J=9.0 Hz), 7.21 (d, 1H, J=2.2 Hz), 7.00 (dd, 1H, J=9.0, 2.2 Hz), 3.81 (s, 3H), 4.08-4.15 (m, 2H), 3.21-3.29 (m, 2H), 2.75 (d, 6H, J=5.0 Hz), 2.13-2.21 (m, 2H). ACPI-MS Found: [M+H]⁺=376.

### Example 4.71

N¹,N¹N-Dimethyl-N³-[2-(5-methoxy-1-methyl-1H-indol-2-yl)-4-quinazolinyl]-1,3-propanediamine dihydrochloride (71). A mixture of 2-(5-methoxy-1-methyl-1H-indol-2-yl)-4(3H)-quinazolinone (C: R═H, R′=5-methoxy-1-methyl-1H-indol-2-yl) (0.308 g, 1.01 mmol) and tetramethylammonium chloride (0.22 g, 2.01 mmol) in POCl₃ (10 mL) was refluxed for 20 min to give the chloroquinazoline (H: R═H, R′=5-methoxy-1-methyl-1H-indol-2-yl) (0.276 g, 84%). The chloroquinazoline (0.226 g, 0.698 mmol) was refluxed with N¹,N¹-dimethyl-1,3-propanediamine (0.26 mL, 2.07 mmol) in dioxane (25 mL) for 2 h, workup and conversion to the hydrochloride salt gave 71 (0.286 g, 89%) as a solid. ¹H NMR (DMSO-d₆) δ ppm 14.3 (bs, 1H), 10.46 (bs, 2H), 8.63 (bd, 1H, J=8.2 Hz), 8.08-8.14 (m, 1H), 8.01 (t, 1H, J=7.7 Hz), 7.67-7.77 (m, 2H), 7.57 (d, 1H, J=9.1 Hz), 7.22 (d, 1H, J=2.4 Hz), 7.06 (dd, 1H, J=9.1, 2.4 Hz), 4.20 (s, 3H), 3.80-3.88 (m, 2H), 3.82 (s, 3H), 3.16-3.22 (m, 2H), 2.75 (d, 6H, J=4.9 Hz), 2.14-2.23 (m, 2H). ACPI-MS Found: [M+H]⁺=390.

### Example 4.72

N³-[2-(1H-Indol-2-yl)-4-quinazolinyl]-N³,N³-dimethyl-1,3-propanediamine dihydrochloride (72). A mixture of 2-(1H-indol-2-yl)-4(3H)-quinazolinone (C: R═H, R′=1H-indol-2-yl) (0.557 g, 2.13 mmol) and tetramethylammonium chloride (0.50 g, 4.56 mmol) in POCl₃ (15 mL) was refluxed for 20 min to give the chloroquinazoline (H: R═H, R′=1H-indol-2-yl) (0.369 g, 62%). The chloroquinazoline (0.282 g, 1.01 mmol) was refluxed with N¹,N¹-dimethyl-1,3-propanediamine (0.38 mL, 3.02 mmol) in dioxane (40 mL) for 2 h, workup and conversion to the hydrochloride salt gave 72 (0.339 g, 80%) as a solid. ¹H NMR (DMSO-d₆) δ ppm 14.5 (bs, 1H), 12.26 (bs, 1H), 10.41 (bs, 2H), 8.54 (bd, 1H, J=7.6 Hz), 7.96-8.18 (m, 3H), 7.64-7.78 (m, 3H), 7.33 (t, 1H, J=7.5 Hz), 7.13 (t, 1H, J=7.5 Hz), 4.06-4.17 (m, 2H), 3.20-3.28 (m, 2H), 2.75 (d, 6H, J=5.0 Hz), 2.12-2.20 (m, 2H). ACPI-MS Found: [M+H]⁺=346.

### Example 4.73

N¹-[2-(1H-Indol-2-yl)-4-quinazolinyl]-N-[3-(4-morpholinyl)propyl]amine dihydrochloride (73). 4-Chloro-2-(1H-indol-2-yl)quinazoline (H: R═H, R′=1H-indol-2-yl) (0.118 g, 0.422 mmol) was refluxed with 3-(4-morpholinyl)propanamine (0.20 mL, 1.36 mmol) in dioxane (15 mL) for 2 h, workup and conversion to the hydrochloride salt gave 73 (0.183 g, 94%) as a solid. ¹H NMR (DMSO-d₆) δ ppm 14.6 (bs, 1H), 12.26 (bs, 1H), 11.04 (bs, 1H), 10.34 (bs, 1H), 8.55 (d, 1H, J=7.8 Hz), 7.95-8.20 (m, 3H), 7.65-7.79 (m, 3H), 7.35 (t, 1H, J=7.6 Hz), 7.14 (t, 1H, J=7.6 Hz), 4.10-4.20 (bd, 2H, J=4.5 Hz), 3.80-3.94 (m, 5H), 3.30-3.48 (m, 3H), 2.97-3.10 (m, 2H), 2.18-2.27 (m, 2H). ACPI-MS Found: [M+H]⁺=388.

### Example 4.74

N¹, N¹-Dimethyl-N³-[2-(1-methyl-1H-indol-2-yl)-4-quinazolinyl]-1,3-propanediamine dihydrochloride (74). A mixture of 2-(1-methyl-1H-indol-2-yl)-4(3H)-quinazolinone (C: R═H, R′=1-methyl-1H-indol-2-yl) (0.251 g, 0.912 mmol) and tetramethylammonium chloride (0.200 g, 1.82 mmol) in POCl₃ (10 mL) was refluxed for 0.5 h to give the chloroquinazoline (H: R═H, R′=1-methyl-1H-indol-2-yl) (0.216 g, 81%). The chloroquinazoline (0.195 g, 0.664 mmol) was refluxed with N¹,N¹-dimethyl-1,3-propanediamine (0.25 mL, 1.99 mmol) in dioxane (25 mL) for 2 h, workup and conversion to the hydrochloride salt gave 74 (0.265 g, 92%) as a solid. ¹H NMR (DMSO-d₆) δ ppm 14.4 (bs, 1H), 10.5 (bs, 2H), 8.66 (bd, 1H, J=7.7 Hz), 8.12 (bd, 1H, J=8.1 Hz), 8.02 (t, 1H, J=7.7 Hz), 7.75-7.83 (m, 2H), 7.73 (t, 1H, J=7.6 Hz), 7.67 (d, 1H, J=8.5 Hz), 7.42 (t, 1H, J=7.5 Hz), 7.20 (t, 1H, J=7.5 Hz), 4.23 (s, 3H), 3.82-3.90 (m, 2H), 3.18-3.26 (m, 2H), 2.75 (d, 1H, J=5.0 Hz), 2.15-2.22 (m, 2H). ACPI-MS Found: [M+H]⁺=360.

### Example 4.75

N¹-[2-(1-Benzothien-2-yl)-4-quinazolinyl]-N³,N³-dimethyl-1,3-propanediamine dihydrochloride (75). A mixture of 2-(1-benzothien-2-yl)-4(3H)-quinazolinone (C: R═H, R′=1-benzothien-2-yl) (1.28 g, 4.60 mmol) in SOCl₂ (50 mL)/dmf (0.1 mL) was refluxed for 45 min to give the chloroquinazoline (H: R═H, R′=1-benzothien-2-yl). The chloroquinazoline was refluxed with N¹,N¹-dimethyl-1,3-propanediamine (1.3 mL, 10.3 mmol) in dioxane (25 mL) for 2 h, workup and conversion to the hydrochloride salt gave 75 (0.265 g, 13%) as a solid. ¹H NMR (DMSO-d₆) δ ppm 10.32 (bs, 1H), 8.88 (bs, 1H), 8.53 (d, 1H, J=7.4 Hz), 8.02-8.15 (m, 3H), 7.97 (t, 1H, J=7.6 Hz), 7.68 (t, 1H, J=7.0 Hz), 7.46-7.57 (m, 2H), 3.79-3.87 (m, 2H), 3.18-3.26 (m, 2H), 2.77 (d, 6H, J=5.0 Hz), 2.15-2.25 (m, 2H). ACPI-MS Found: [M+H]⁺=363.

### Example 4.76

N¹,N¹-Dimethyl-N³-[2-(3-quinolinyl)-4-quinazolinyl]-1,3-propanediamine dihydrochloride (76). A mixture of 2-(3-quinolinyl)-4(3H)-quinazolinone (C: R=5-Me, R′=3-quinolinyl) (0.490 g, 1.80 mmol) in SOCl₂ (7 mL)/DMF (0.1 mL) was refluxed for 10 min. to give the chloroquinazoline (H: R═H, R′=3-quinolinyl). The chloroquinazoline was refluxed with N¹,N¹-dimethyl-1,3-propanediamine (0.68 mL, 5.40 mmol) in dioxane (25 mL) for 2 h, workup and conversion to the hydrochloride salt gave 76 (0.688 g, 82%) as a solid. ¹H NMR (DMSO-d₆) δ ppm 14.8 (bs, 1H), 10.71 (bs, 1H), 10.60 (bs, 1H), 9.90 (d, 1H, J=2.1 Hz), 9.77 (s, 1H), 8.75 (d, 1H, J=8.2 Hz), 8.29-8.38 (m, 2H), 8.22 (d, 1H, J=8.5 Hz), 7.97-8.09 (m, 2H), 7.83 (t, 1H, J=7.6 Hz), 7.78 (t, 1H, J=7.6 Hz), 3.96-4.03 (m, 2H), 3.22-3.30 (m, 2H), 2.75 (d, 6H, J=4.9 Hz), 2.22-2.32 (m, 2H). ACPI-MS Found: [M+H]⁺=358.

### Example 4.77

N¹N¹-Dimethyl-N³-[2-(2-naphthyl)-4-quinazolinyl]-1,3-propanediamine dihydrochloride (77). A mixture of 2-(2-naphthyl)-4(3H)-quinazolinone (C: R═H, R′=2-naphthyl) (0.378 g, 1.39 mmol) in SOCl₂ (5 mL)/dmf (0.1 mL) was refluxed for 1 h to give the chloroquinazoline (H: R═H, R′=2-naphthyl). The chloroquinazoline was refluxed with N¹,N¹-dimethyl-1,3-propanediamine (0.52 mL, 4.13 mmol) in dioxane (25 mL) for 2 h, workup and conversion to the hydrochloride salt gave 77 (0.471 g, 60%) as a solid. ¹H NMR (DMSO-d₆) δ ppm 14.6 (b, 1H), 10.61 (d, 2H), 9.21 (s, 1H), 8.70 (d, 1H, J=8.2 Hz), 8.54 (dd, 1H, J=8.7, 1.3 Hz), 8.29 (d, 1 h, J=8.2 Hz), 8.23 (d, 1H, J=7.8 Hz), 8.18 (d, 1H, J=8.7 Hz), 8.00-8.11 (m, 2H), 7.65-7.80 (m, 3H), 3.95-4.01 (m, 2H), 3.22-3.30 (m, 2H), 2.77 (d, 6H, J=5.0 Hz), 2.21-2.30 (m, 2H). ACPI-MS Found: [M+H]⁺=357.

### Example 4.78

2-(1-Benzofuran-2-yl)-N³-[2-(1-methyl-2-pyrrolidinyl)ethyl]-4-quinazolinamine dihydrochloride (78). A solution of 2-(1-benzofuran-2-yl)-4-chloroquinazoline (H: R═H, R′=benzofuran-2-yl) (0.163 g, 0.581 mmol) and 2-(1-methyl-2-pyrrolidinyl)etheneamine (0.25 mL, 1.38 mmol) in dioxane (15 mL) was refluxed for 2 h, workup and conversion to the hydrochloride salt gave 78 (0.223 g, 86%) as a solid. ¹H NMR (DMSO-d₆) δ ppm 14.5 (bs, 1H), 10.80 (bs, 1H), 10.35 (bs, 1H), 8.66 (d, 1H, J=8.2 Hz), 8.33 (s, 1H), 8.12 (d, 1H, J=8.3 Hz), 8.01 (t, 1H, J=7.5 Hz), 7.91 (d, 1H, J=7.7 Hz), 7.81 (dd, 1H, J=8.3, 0.5 Hz), 7.73 (t, 1H, J=7.4 Hz), 7.59 (td, 1H, J=7.8, 1.1 Hz), 7.44 (td, 1H, J=7.3, 0.6 Hz), 3.89-3.98 (m, 2H), 3.48-3.60 (m, 2H), 2.96-3.06 (m, 1H), 2.77 (d, 3H, J=5.0 Hz), 2.41-2.51 (m, 2H), 2.09-2.20 (m, 1H), 1.90-2.00 (m, 2H), 1.78-1.88 (m, 1H). ACPI-MS Found: [M+H]⁺=373.

### Example 4.79

2-(1-Benzofuran-2-yl)-7,8-dimethyl-N-[2-(1-methyl-2-pyrrolidinyl)ethyl]-4-quinazolinamine dihydrochloride (79). A mixture of 2-(1-benzofuran-2-yl)-7,8-dimethyl-4(3H)-quinazolinone (C: R=7,8-diMe, R′=benzofuran-2-yl) (0.104 g, 0.358 mmol) and tetramethylammonium chloride (0.080 g, 0.730 mmol) in POCl₃ (10 mL) was refluxed for 0.5 h to give the chloroquinazoline (H: R=7,8-diMe, R′=benzofuran-2-yl). The chloroquinazoline was refluxed with 2-(1-methyl-2-pyrrolidinyl)etheneamine (0.156 mL, 1.08 mmol) in dioxane (40 mL) for 2 h, workup and conversion to the hydrochloride salt gave 79 (0.095 g, 56%) as a solid. ¹H NMR (DMSO-d₆) δ ppm 10.16 (s, 1H), 8.50 (s, 1H), 8.04 (d, 1H, J=8.4 Hz), 7.70-7.82 (m, 3H), 7.43 (td, 1H, J=7.7, 1.3 Hz), 7.38 (d, 1H, J=8.4 Hz), 7.33 (td, 1H, J=7.5, 0.8 Hz), 3.50-3.56 (m, 2H), 3.29-3.40 (m, 1H), 2.98-3.08 (m, 1H), 2.80 (d, 3H, J=5.0 Hz), 2.63-2.69 (m, 1H), 2.64 (s, 3H), 2.44 (s, 3H), 2.32-2.50 (m, 3H), 1.79-2.10 (m, 4H). ACPI-MS Found: [M+H]⁺=397.

### Example 5.1

N¹-[2-(1-benzofuran-2-yl)-4-quinolinyl]-N³,N³-dimethyl-1,3-propanediamine dihydrochloride (80) (Scheme 5).

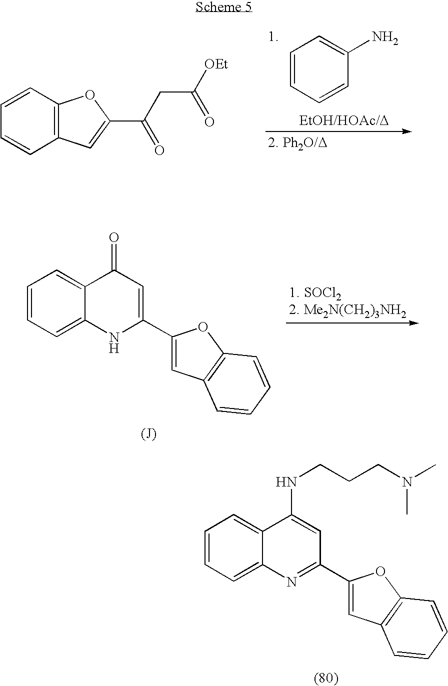

A solution of ethyl 3-benzofuran-2-yl-3-oxopropionate (3.9 g, 16.8 mmol) and aniline (1.53 mL, 16.8 mmol) in ethanol (150 mL)/acetic acid (0.5 mL) was heated at 54° C. for 24 h, acetic acid (0.5 mL) was added and the mixture was then refluxed at 75° C. for 2 days. The solvent was removed in vacuo and the residue was refluxed in diphenyl ether for 20 min., the mixture was cooled and the solid was filtered off and washed with chloroform to give 4(1H)-quinolinone (J) (740 mg, 74%) which was used in the subsequent step without any further purification. A mixture of (J) in thionyl chloride (10 mL) was refluxed for 30 min and then excess thionyl chloride was removed in vacuo. The chloro compound and 3-dimethylaminopropylamine (1.5 mL, 12 mmol) in dioxane (20 mL) was refluxed for 3 h, 3-dimethylaminopropylamine (2 mL, 16 mmol) was added and the mixture was refluxed for a further 1 h. The solvent was removed in vacuo and the residue was partitioned between EtOAc/brine. Removal of the solvent from the organic fraction gave a solid which was purified by HPLC and converted to its HCl salt, to give 80 (24 mg) as a solid. ¹H NMR (DMSO-d₆) δ ppm 14.02 (bs, 1H), 10.36 (bs, 2H), 9.40 (bs, 1H), 8.61 (bd, 1H, J=8.0 Hz), 8.51 (bs, 1H), 8.28 (bd, 1H, J=7.9 Hz), 7.97 (bt, 1H, J=7.6 Hz), 7.91 (d, 1H, J=7.8 Hz), 7.82 (d, 1H, J=8.4 Hz), 7.71 (t, 1H, J=7.5 Hz), 7.60 (t, 1H, J=7.8 Hz), 7.38-7.48 (m, 2H), 3.76-3.84 (m, 2H), 3.18-3.28 (m, 2H), 2.78 (d, 6H, J=4.8 Hz), 2.13-2.22 (m, 2H). ACPI-MS Found: [M+H]⁺=346.

### Example 6.1

N¹-[3-(1-benzofuran-2-yl)-1-isoquinolinyl]-N³,N³-dimethyl-1,3-propanediamine dihydrochloride (81) (Scheme 6).

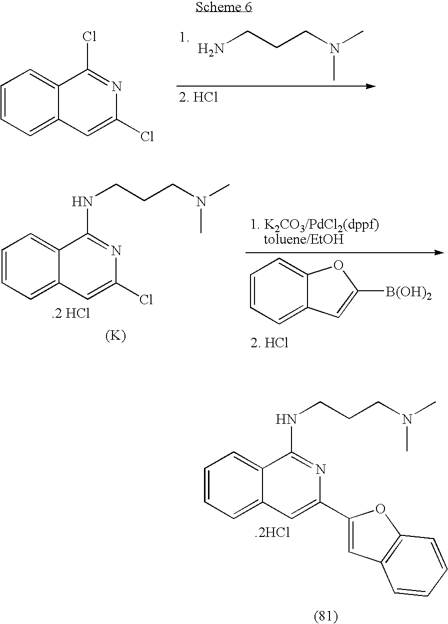

N¹-(3-chloro-1-isoquinolinyl)-N³,N³-dimethyl-1,3-propanediamine dihydrochloride (K). 1,3-Dichloroisoquinoline (1.00 g, 5.05 mmol) and N,N-dimethyl-1,3-propanediamine (2.0 mL) were heard to reflux in a sealed tube for 0.5 h. The mixture was quenched with water and extracted with EtOAc. The solvent was removed in vacuo and the residue was dissolved in MeOH and treated with HCl in MeOH (1.25 M, 20 mL). The solvent was removed in vacuo and the compound was recrystallised from MeOH/acetone to give K (1.682 g, 99%) as a microcrystalline solid. ¹H NMR (DMSO-d₆) δ ppm 10.30 (bs, 1H), 8.29 (d, 1H, J=8.4 Hz), 8.06 (bs, 1H), 7.62-7.70 (m, 2H), 7.49 (ddd, 1H, J=8.3, 6.5, 1.6 Hz), 6.99 9 s, 1H), 3.54 (t, 2H, J=6.6 Hz), 3.10-3.18 (m, 2H), 2.76 (d, 6H, J=5.0 Hz), 2.00-2.09 (m, 2H). ACPI-MS Found: [M+H]⁺=266, 264.

N¹-[3-(1-benzofuran-2-yl)-1-isoquinolinyl]-N³,N³-dimethyl-1,3-propanediamine dihydrochloride (81). A mixture of N¹-(3-chloro-1-isoquinolinyl)-N³,N³-dimethyl-1,3-propanediamine dihydrochloride (K) (0.499 g, 1.66 mmol) and 1-benzofuran-2-ylboronic acid (0.323 g, 1.99 mmol) in toluene (50 mL)/EtOH (30 mL)/aqueous K₂CO₃ (2 M, 10 mL) was purged with nitrogen. PdCl₂(dppf) was added and the mixture was refluxed under nitrogen for 2 h then partitioned between EtOAc/water. Column chromatography (EtOAc+1% aq. NH₃) gave a product which contained small amounts of chloroisoquinoline starting material. The reaction was performed again on the product derived from column chromatography. The product obtained from the second coupling reaction was dissolved in MeOH and treated with HCl in MeOH (20 mL, 1.25 M). Recrystallisation from MeOH/EtOAc gave 81 (0.247 g, 36%) as a solid. ¹H NMR (DMSO-d₆) 8 ppm 10.34 (bs, 1H), 8.34 (d, 1H, J=8.3 Hz), 7.87 (d, 1H, J=7.8 Hz), 7.64-7.75 (m, 3H), 7.51-7.58 (m, 3H), 7.36 (td, 1H, J=7.3, 1.3 Hz), 7.29 (td, 1H, J=7.6, 0.9 Hz), 3.73 (bt, 2H, J=6.5 Hz), 3.17-3.24 (m, 2H), 2.76 (d, 6H, J=4.9 Hz), 2.12-2.21 (m, 2H). ACPI-MS Found: [M+H]⁺=346.

TABLE 1
Details of compounds representative of the invention

| | |
|---|---|
| | A |
| 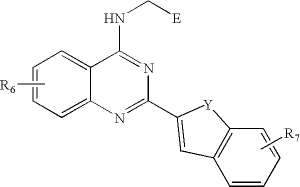 | |
| | B |
| 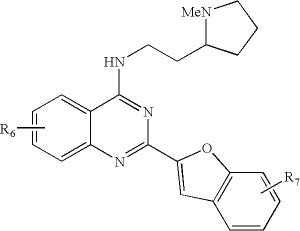 | |
| | C |
| 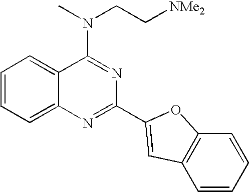 | |
| | D |
| 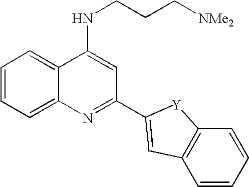 | |
| | E |
| 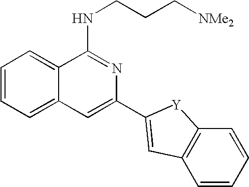 | |

| No | Fm | R₆ | R₇ | Y | E |
|---|---|---|---|---|---|
| 1 | A | H | H | O | CH₂NMe₂ |
| 2 | C | — | — | — | — |
| 3 | A | H | H | O | (CH₂)₂NMe₂ |
| 4 | A | H | N | O | (CH₂)₃NMe₂ |
| 5 | A | H | H | O | (CH₂)₂NEt₂ |
| 6 | A | H | H | O | (CH₂)₂NPr₂ |
| 7 | A | H | H | O | (CH₂)₂N[(CH₂)₂OH]₂ |
| 8 | A | H | H | O | (CH₂)₂Nmorph |
| 9 | A | H | H | O | (CH₂)₂[4-Mepipz] |
| 10 | A | H | H | O | (CH₂)₂[pyrrolidine] |
| 11 | A | H | H | O | (CH₂)₂NH(cyclopropyl) |
| 12 | A | H | H | O | (CH₂)₂NHMe |
| 13 | A | H | H | O | (CH₂)₂NHEt |
| 14 | A | H | H | O | C(Me₂)CH₂NMe₂ |
| 15 | A | 5-aza | H | O | (CH₂)₂NMe₂ |
| 16 | A | 5-Me | H | O | (CH₂)₂NMe₂ |
| 17 | A | 5-OMe | H | O | (CH₂)₂NMe₂ |
| 18 | A | 5-Cl | H | O | (CH₂)₂NMe₂ |
| 19 | A | 5-NO₂ | H | O | (CH₂)₂NMe₂ |
| 20 | A | 5-NH₂ | H | O | (CH₂)₂NMe₂ |
| 21 | A | 5-CONH(CH₂)₃NMe₂ | H | O | (CH₂)₂NMe₂ |
| 22 | A | 6-aza | H | O | (CH₂)₂NMe₂ |
| 23 | A | 6-Me | H | O | (CH₂)₂NMe₂ |
| 24 | A | 6-CF₃ | H | O | (CH₂)₂NMe₂ |
| 25 | A | 6-OMe | H | O | (CH₂)₂NMe₂ |
| 26 | A | 6-F | H | O | (CH₂)₂NMe₂ |
| 27 | A | 6-Cl | H | O | (CH₂)₂NMe₂ |
| 28 | A | 6-Br | H | O | (CH₂)₂NMe₂ |
| 29 | A | 6-NO₂ | H | O | (CH₂)₂NMe₂ |
| 30 | A | 6-NH₂ | H | O | (CH₂)₂NMe₂ |
| 31 | A | 6-CN | H | O | (CH₂)₂NMe₂ |
| 32 | A | 6-CONH₂ | H | O | (CH₂)₂NMe₂ |
| 33 | A | 7-aza | H | O | (CH₂)₂NMe₂ |
| 34 | A | 7-Me | H | O | (CH₂)₂NMe₂ |
| 35 | A | 7-CF₃ | H | O | (CH₂)₂NMe₂ |
| 36 | A | 7-OMe | H | O | (CH₂)₂NMe₂ |
| 37 | A | 7-F | H | O | (CH₂)₂NMe₂ |
| 38 | A | 7-Cl | H | O | (CH₂)₂NMe₂ |
| 39 | A | 7-Br | H | O | (CH₂)₂NMe₂ |
| 40 | A | 7-NO₂ | H | O | (CH₂)₂NMe₂ |
| 41 | A | 7-NH₂ | H | O | (CH₂)₂NMe₂ |
| 42 | A | 7-CN | H | O | (CH₂)₂NMe₂ |
| 43 | A | 7-CONH₂ | H | O | (CH₂)₂NMe₂ |
| 44 | A | 8-aza | H | O | (CH₂)₂NMe₂ |
| 45 | A | 8-Me | H | O | (CH₂)₂NMe₂ |
| 46 | A | 8-Ph | H | O | (CH₂)₂NMe₂ |
| 47 | A | 8-CF₃ | H | O | (CH₂)₂NMe₂ |
| 48 | A | 8-OMe | H | O | (CH₂)₂NMe₂ |
| 49 | A | 8-Cl | H | O | (CH₂)₂NMe₂ |
| 50 | A | 8-NO₂ | H | O | (CH₂)₂NMe₂ |
| 51 | A | 8-NH₂ | H | O | (CH₂)₂NMe₂ |
| 52 | A | 8-CN | H | O | (CH₂)₂NMe₂ |
| 53 | A | 8-CONH₂ | H | O | (CH₂)₂NMe₂ |
| 54 | A | 6,7-benz | H | O | (CH₂)₂NMe₂ |
| 55 | A | 6,7-diCl | H | O | (CH₂)₂NMe₂ |
| 56 | A | 6,8-diCl | H | O | (CH₂)₂NMe₂ |
| 57 | A | 6,8-diBr | H | O | (CH₂)₂NMe₂ |
| 58 | A | 7,8-diMe | H | O | (CH₂)₂NMe₂ |
| 59 | A | 7,8-diOMe | H | O | (CH₂)₂NMe₂ |
| 60 | A | H | 3′-Me | O | (CH₂)₂NMe₂ |
| 61 | A | H | 4′-Cl, 5′-OMe, | O | (CH₂)₂NMe₂ |
| 62 | A | H | 5′-OMe | O | (CH₂)₂NMe₂ |
| 63 | A | H | 5′-Me | O | (CH₂)₂NMe₂ |
| 64 | A | H | 5′-Cl | O | (CH₂)₂NMe₂ |
| 65 | A | H | 5′-Br | O | (CH₂)₂NMe₂ |
| 66 | A | H | 6′-OMe | O | (CH₂)₂NMe₂ |
| 67 | A | H | 7′-Me | O | (CH₂)₂NMe₂ |
| 68 | A | H | 7′-OMe | O | (CH₂)₂NMe₂ |
| 69 | A | 8-Me | 3′-Me | O | (CH₂)₂NMe₂ |
| 70 | A | H | 5′-OMe | NH | (CH₂)₂NMe₂ |
| 71 | A | H | 5′-OMe | NMe | (CH₂)₂NMe₂ |
| 72 | A | H | H | NH | (CH₂)₂NMe₂ |
| 73 | A | H | H | NH | (CH₂)Nmorph |
| 74 | A | H | H | NMe | (CH₂)₂NMe₂ |
| 75 | A | H | H | S | (CH₂)₂NMe₂ |
| 76 | A | H | H | CH═N | (CH₂)₂NMe₂ |
| 77 | A | H | H | CH═CH | (CH₂)₂NMe₂ |
| 78 | B | H | H | — | — |
| 79 | B | 7,8-diMe | H | — | — |
| 80 | D | — | — | — | — |
| 81 | E | — | — | — | — |

### Biological Activity of Compounds of the Invention

Description of the in vitro assay: This protocol employs a novel assay that was used to measure the restoration of one of the principal p53 functions, that of regulating entry into S-phase (DNA replication) of the cell division cycle. Without being bound to any specific understanding, it is thought that these compounds may act to restore the known ability of p53 to induce cell cycle arrest in the G₁-phase of the cell division cycle through induction of the p21^{WAF1} protein in response to DNA damage (Levine. Cell 1997, 88, 323-31). Logarithmic phase cultures of tumour cell lines are irradiated in the presence of an inhibitor of cell division (to prevent the generation of G₁-phase cells by cell division), and then allowed to grow for approximately one cell division time. If p53 function is present it will inhibit the progression of cells from the G₁-phase to the S-phase of the cell division cycle, as a consequence of induction of the p21^{WAF1} protein. If p53 function is absent there will be little or no cells in the G₁-phase of the cell division cycle at the end of the incubation. If p53 function is completely restored, the proportion of cells in the G₁-phase of the cell division cycle will approximate that of cells that have not been irradiated. The ability of an individual drug to restore p53 function is therefore evaluated against a positive control of non-irradiated cells and a negative control of cells that have been treated with a combination of radiation and a mitotic inhibitor.

Logarithmic phase cultures of tumour cell lines are plated in insulin-transferrin-selenite growth medium on 100 mm plates (10 ml) at a density of 10⁴ cells/ml, using the standard cell culture conditions that are established in this laboratory (Marshall et al., Oncol Res 1994, 5, 301-9). The test compound is added to some cultures at a range of concentrations up to 20 μM. The anticancer drug paclitaxel is used as an inhibitor of cell division and is added to some cultures at a concentration of 200 nM. Cultures are irradiated where indicated at a dose of 9 Gray. Following irradiation, cultures are incubated at 37° C. for 24 hours, the cells harvested, washed once and fixed overnight in 100% methanol at −20° C. at a density of 5×10⁵ cells/ml. Cells are rehydrated by washing in phosphate buffered saline with 2% foetal bovine serum, then incubated with RNAase A (0.25 mg/ml) at 37° C. for 30 min. Cells are then washed and resuspended in phosphate buffered saline containing 0.1 mM EDTA and 25 μg/ml propidium iodide. Cycle analysis (red fluorescence) is performed using a Becton Dickinson (Mountain View, Calif.) FACScan flow cytometer. Cellular DNA content profiles are analyzed using Modfit software (Verity Software House, Inc.) to provide estimates of the proportions of G₁-, S- and G₂/M-phase cells, and of other cellular material. By comparing the effects of irradiation and addition of paclitaxel separately, the assay provides evidence of non-specific inhibition of cell growth from changes in cell number, cell cycle distribution, and production of cellular debris.

Using cell cultures that have been both irradiated and treated with paclitaxel, the proportion of cells in G₁-phase is plotted against the concentration of added test compound. Depending on the cell line, the proportion of G₁-phase cells in the absence of added compound is generally less than 5% (defined as a). Increasing concentrations of active compound raise the proportion to a level comparable to that in control cells that have received no radiation or paclitaxel (generally around 40% and defined as b). The 50%-activating concentration (AC-50%) of the test compound is defined as the concentration that restores the G₁-phase proportion of the cultured cells to a value of (a+b)/2.

Multiple control experiments were carried out to ensure that the compounds did not cause G₁-phase arrest when administered without irradiation or without addition of paclitaxel. Other experiments carried out with a number of cell lines showed that wild-type p53 protein was necessary for radiation to cause G₁-phase arrest in the absence of drug.

The NZOV11 human ovarian cell line, previously developed in this laboratory according to methods that have previously been published (Baguley B C et al., Eur J Cancer 1998, 34, 1086), was used in these studies to compare the activity of each of the drugs. The p53 protein in this cell line is mutated and inactive as a result of a mutation of the aminoacid at position 248 from arginine to glutamine. The AC-50% values for some of these compounds are shown in Table 2. Other studies have established that compound 3 is able to restore p53 function in a number of other cell lines with mutations in other parts of the p53 protein.

TABLE 2
Biological activity of selected compounds of Table 1.

| No | Activity | AC-50% μM |
|---|---|---|
| 3 | Active | 8.5 |
| 29 | Active | 12.0 |
| 30 | Active | 9.5 |
| 35 | Active | 3.4 |
| 45 | Active | 5.8 |
| 58 | Active | 2.7 |
| 60 | Active | 18.0 |
| 75 | Active | 8.0 |
| 79 | Active | 7.5 |
| 80 | Active | 15.0 |

Footnote for Table 2
AC-50% is the concentration of drug that restores the G₁-phase proportion of the cultured cells as defined above.

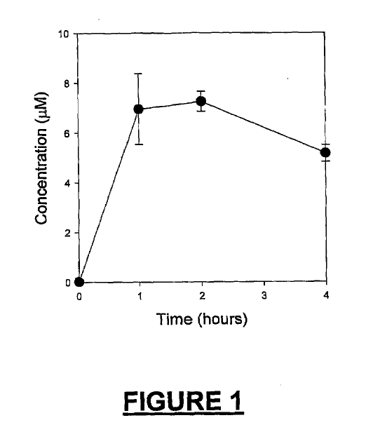

Pharmacological studies in mice: A further consideration in this project is whether effective plasma concentrations can be achieved in vivo without significant side-effects. C57Bl mice were maintained under standard conditions in accordance with institutional ethical guidelines. The maximum tolerated intraperitoneal single dose of compound 3 was determined by administering different doses of drug to mice and found to be 100 mg/kg. No signs of toxicity, such as weight loss, ruffling of fur or behavioral changes, were observed following administration of this dose. An effective analytical procedure for the determination of concentrations of 3 in mouse plasma was developed, using high performance liquid chromatography and detection by ion trap mass spectrometry. The method is broadly applicable to compounds in the series. Blood samples were obtained under terminal anesthesia either before or at 1, 2 and 4 hours after a single intraperitoneal dose of 100 mg/kg. Plasma was prepared and analyzed using the method developed above. As shown in FIG. 1, the achieved plasma concentrations of compound 3 following a single intraperitoneal administration (100 mg/kg) to C57Bl mice were comparable to the AC-50% value for in vitro reconstitution of p53 activity.

In vivo estimation of ability to restore p53 function: A further consideration in this project is to determine whether members of this series have the ability to inhibit the growth of human cell lines that contain mutant p53 protein. This consideration was addressed with the use of compound 3, for which in vitro evidence has been obtained for ability to restore p53 function (Table 2) and for which pharmacological evidence has been obtained for biologically relevant plasma concentrations in vivo in the absence of significant side-effects (FIG. 1). Immunodeficient (rag 1) mice were maintained under standard conditions in accordance with institutional ethical guidelines. Cells from the NZM4 cell line (Marshall E S et al., Eur J Cancer 1994; 30A: 1370-1376) were grown in culture and harvested. Mice were injected subcutaneously with 10⁷ cells and tumours allowed to grow to a diameter of approximately 5 mm. Mice received whole-body irradiation of 2 Gray and were then administered compound 3 by intraperitoneal injection immediately after, and 1 and 2 days following, irradiation. No signs of toxicity were evident. Control animals received no treatment or were irradiated in the absence of drug administration. In a separate experiment, tumours growing in rag1 mice treated with drug alone at this schedule were found to grow at the identical rate to those in mice that had not been treated with drug.

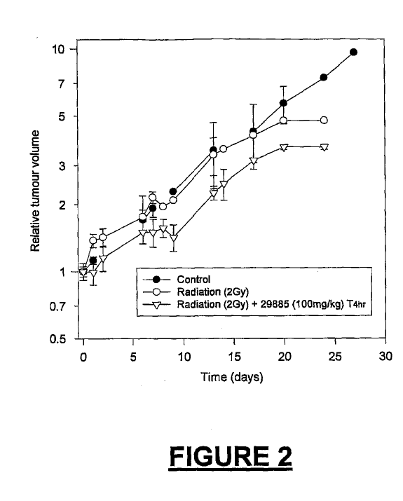

Tumour growth was recorded three times weekly by measuring the minor and major dimensions of the tumour and tumour volumes were calculated as 0.52 times (minor dimension)² times (major dimension). Tumour volumes were normalized to the initial volume and plotted versus time. FIG. 2 illustrates the growth curves for immunodeficient mice with NZM4 human tumour xenografts. Mice were either untreated (closed circles), treated with 2 Gray radiation alone (open circles) or treated with radiation combined with compound 3 (100 mg/kg per dose). As shown in FIG. 2, tumours untreated mice or mice receiving irradiation alone (2 Gray) grew at similar rates, with a time to reach three times the initial tumour volume of 12 days. Tumours from mice receiving radiation (2 Gray) together with compound 3 on days 0, 1 and 2 days after irradiation grew more slowly with (2 Gray) with a time to reach three times the initial tumour volume of 17 days.

Wherein the foregoing description reference has been made to reagents, or integers having known equivalents thereof, then those equivalents are herein incorporated as if individually set forth.

While this invention has been described with reference to certain embodiments and examples, it is to be appreciated that further modifications and variations can be made to embodiments and examples without departing from the spirit or scope of the invention.

Throughout this specification, unless the context requires otherwise, the words “comprise”, “comprising” and the like, are to be construed in an inclusive sense as opposed to an exclusive sense, that is to say, in the sense of “including, but not limited to”.

The reference to any prior art in this specification is not, and should not be taken as, an acknowledgment or any form of suggestion that that prior art forms part of the common general knowledge in New Zealand.

1. A compound of Formula (I): 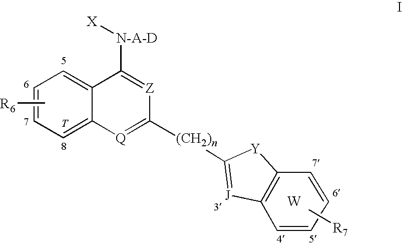
wherein;
D is selected from NR₁R₂ where R₁ and R₂ each independently represent H, lower C1-C6 alkyl or cycloalkyl optionally substituted with amino, hydroxyl or methoxy groups, or with one or more oxygen or nitrogen atoms as part of the cycloalkyl structure represents morpholine, pyrrolidine, piperidine, imidazole or 4-methylpiperazine;
n is selected from 0, 1 or 2;
X is selected from H or lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine;
Y is selected from O, CHR₃, S or, NR₄, where R₃ and R₄ each independently represent H or lower, C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine;
Z and Q represent N or CH, with the proviso that at least one of them is N;
J represents N or CR₅; where R₅ represents H or lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine,
A is (CH₂)ₙ where n is from 2 to 6, or A together with D forms a ring structure R₆ and R₇ at one or more of the available positions on rings T and W respectively, at each occurrence independently represent one or more H, halogen, C1-C4 alkyl, C1-C4 alkenyl, C1-C4 alkynyl, OR₈, SR₈, NR₈R₉, CH₂R₈, COR₈, SOR₈, SO₂R₈, SO₂NR₈R₉, CO₂R₈, CONR₈R₉, CF₃, CN, or NO₂, where R₈ and R₉ each independently represent H, lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine,
or a physiologically acceptable salt or phosphate prodrug or carboxylic acid or aminoacid ester prodrug thereof;
with the proviso that the compound 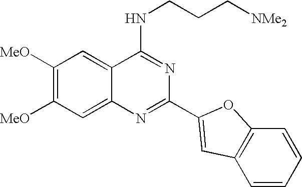
is excluded.

2. A compound according to claim 1 wherein:
Z represents N or CH, and Q represents N.

3. A compound according to claim 1 wherein A together with D form a ring structure wherein the ring structure is: 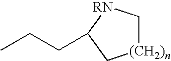
wherein n is from 1 to 4 and R represents a branched or unbranched C₁-C₆ alkyl.

4. A compound according to claim 1 wherein A together with D form a ring structure wherein the ring structure is:

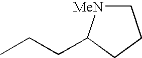

5. A compound according to claim 2 wherein:
D is NR₁R₂ where R₁ and R₂ each independently represent H, lower C1-C6 alkyl or cycloalkyl, where one or more oxygen or nitrogen atoms as part of the cycloalkyl structure may represent azetidine, pyrrolidine, piperidine, piperazine or morpholine.
n is selected from be 0 or 1;
X is selected from H or lower C1-C6 alkyl or cycloalkyl;
Y represents O or S;
both Z and Q are N;
J represents CH or C-Me;
A is (CH₂)ₙ where n is from 2 to 4, or A together with D form a ring structure;
R₆ and R₇ at the 6-, 7- or 8-positions on ring T and at the 3′-position on ring W respectively, at each occurrence independently represent one or more H, halogen, C1-C4 alkyl, C1-C4 alkenyl, C1-C4 alkynyl, SR₈, NR₈R₉, CH₂R₈, COR₈, SOR₈, SO₂R₈, SO₂NR₈R₉, CO₂R₈, CONR₈R₉, CF₃, CN, or NO₂, where R₈ and R₉ each independently represent H or lower C1-C6 alkyl or cycloalkyl optionally substituted with amino, hydroxyl or methoxy groups;
or a physiologically acceptable salt or phosphate prodrug or carboxylic acid or aminoacid ester prodrug thereof.

6. A compound according to claim 2 wherein:
D is NR₁R₂ where R₁ and R₂ each independently represent H or lower C1-C6 alkyl or cycloalkyl;
n is 0;
X is H;
Y is O;
both Z and Q are N;
J is CH;
A is (CH₂)₃;
R₆ and R₇ at the 6-, 7- or 8-positions on ring T and at the 3′ positions on ring W respectively, at each occurrence independently represent one or more H, halogen, C1-C4 alkyl, CF₃, NO₂ and NH₂— or
a physiologically acceptable salt or phosphate prodrug or carboxylic acid or aminoacid ester prodrug thereof.

7. A compound according to claim 1 wherein the compound is a salt.

8. A compound according to claim 7 wherein the compound is a hydrochloride salt.

9. A compound according to claim 1 wherein Formula I represents one of the following:
N¹-[2-(1-benzofuran-2-yl)-4-quinazolinyl]-N²,N²-dimethyl-1,2-ethanediamine;
N¹-[2-(1-benzofuran-2-yl)-4-quinazolinyl]-N¹,N²N²-trimethyl-1,2-ethanediamine
N¹-[2-(1-benzofuran-2-yl)-4-quinazolinyl]-N³,N³-dimethyl-1,3-propanediamine dihydrochloride;
N¹-[2-(1-benzofuran-2-yl)-4-quinazolinyl]-N⁴,N⁴-dimethyl-1,4-butanediamine dihydrochloride;
N¹-[2-(1-benzofuran-2-yl)-4-quinazolinyl]-N³,N³-diethyl-1,3-propanediamine dihydrochloride;
N¹-[2-(1-benzofuran-2-yl)-4-quinazolinyl]-N³,N³-dipropyl-1,3-propanediamine;
N¹-[2-(1-benzofuran-2-yl)-4-quinazolinyl]-N³,N³-bis(2-hydroxyethyl)-1,3-propanediamine;
2-(1-benzofuran-2-yl)-N-[3-(4-morpholinyl)propyl]-4-quinazolinamine dihydrochloride;
2-(1-benzofuran-2-yl)-N-[3-(4-methyl-1-piperazinyl)propyl]-4-quinazolinamine;
2-(1-benzofuran-2-yl)-N-[3-(1-pyrrolidinyl)propyl]-4-quinazolinamine dihydrochloride;
N¹-[2-(1-benzofuran-2-yl)-4-quinazolinyl]-N³-cyclopropyl-1,3-propanediamine dihydrochloride;
N¹-[2-(1-benzofuran-2-yl)-4-quinazolinyl]-N³-methyl-1,3-propanediamine dihydrochloride;
N¹-[2-(1-benzofuran-2-yl)-4-quinazolinyl]-N³-ethyl-1,3-propanediamine dihydrochloride;
N¹-[2-(1-benzofuran-2-yl)-4-quinazolinyl]-N³, N³,2,2-tetramethyl-1,3-propanediamine dihydrochloride;
N¹-[2-(1-benzofuran-2-yl)pyrido[3,2-d]pyrimidin-4-yl]-N³,N³-dimethyl-1,3-propanediamine dihydrochloride;
N¹-[2-(1-benzofuran-2-yl)-5-methyl-4-quinazolinyl]-N³,N³-dimethyl-1,3-propanediamine dihydrochloride;
N¹-[2-(1-benzofuran-2-yl)-5-methoxy-4-quinazolinyl]-N³,N³-dimethyl-1,3-propanediamine dihydrochloride;
N¹-[2-(1-benzofuran-2-yl)-5-chloro-4-quinazolinyl]-N³,N³-dimethyl-1,3-propanediamine dihydrochloride;
N¹-[2-(1-benzofuran-2-yl)-5-nitro-4-quinazolinyl]-N³,N³-dimethyl-1,3-propanediamine;
N¹-[2-(1-benzofuran-2-yl)-N⁴-[3-(dimethylamino)propyl]-4,5-quinazolinediamine dihydrochloride;
2-(1-benzofuran-2-yl)-N-[3-(dimethylamino)propyl]-4-{[3-(dimethylamino)propyl]amino}-5-quinazolinecarboxamide;
N¹-[2-(1-benzofuran-2-yl)pyrido[4,3-d]pyrimidin-4-yl]-N³,N³-dimethyl-1,3-propanediamine;
N¹-[2-(1-benzofuran-2-yl)-6-methyl-4-quinazolinyl]-N³,N³-dimethyl-1,3-propanediamine dihydrochloride;
N¹-[2-(1-benzofuran-2-yl)-6-(trifluoromethyl)-4-quinazolinyl]-N³,N³-dimethyl-1,3-propanediamine;
N¹-[2-(1-benzofuran-2-yl)-6-methoxy-4-quinazolinyl]-N³,N³-dimethyl-1,3-propanediamine dihydrochloride;
N¹-[2-(1-benzofuran-2-yl)-6-fluoro-4-quinazolinyl]-N³,N³-dimethyl-1,3-propanediamine;
N¹-[2-(1-benzofuran-2-yl)-6-chloro-4-quinazolinyl]-N³,N³-dimethyl-1,3-propanediamine dihydrochloride;
N¹-[2-(1-benzofuran-2-yl)-6-bromo-4-quinazolinyl]-N³,N³-dimethyl-1,3-propanediamine dihydrochloride;
N¹-[2-(1-benzofuran-2-yl)-6-nitro-4-quinazolinyl]-N³,N³-dimethyl-1,3-propanediamine;
N¹-[2-(1-benzofuran-2-yl)-N⁴-[3-(dimethylamino)propyl]-4,6-quinazolinediamine dihydrochloride;
2-(1-benzofuran-2-yl)-4-{[3-(dimethylamino)propyl]amino}-6-quinazolinecarbonitrile;
2-(1-benzofuran-2-yl)-4-{[3-(dimethylamino)propyl]amino}-6-quinazolinecarboxamide dihydrochloride;
N¹-[2-(1-benzofuran-2-yl)pyrido[3,4-d]pyrimidin-4-yl]-N³,N³-dimethyl-1,3-propanediamine dihydrochloride;
N¹-[2-(1-benzofuran-2-yl)-7-methyl-4-quinazolinyl]-N³,N³-dimethyl-1,3-propanediamine dihydrochloride;
N¹-[2-(1-benzofuran-2-yl)-7-(trifluoromethyl)-4-quinazolinyl]-N³,N³-dimethyl-1,3-propanediamine dihydrochloride;
N¹-[2-(1-benzofuran-2-yl)-7-methoxy-4-quinazolinyl]-N³,N³-dimethyl-1,3-propanediamine dihydrochloride;
N¹-[2-(1-benzofuran-2-yl)-7-fluoro-4-quinazolinyl]-N³,N³-dimethyl-1,3-propanediamine dihydrochloride;
N¹-[2-(1-benzofuran-2-yl)-7-chloro-4-quinazolinyl]-N³,N³-dimethyl-1,3-propanediamine dihydrochloride;
N¹-[2-(1-benzofuran-2-yl)-7-bromo-4-quinazolinyl]-N³,N³-dimethyl-1,3-propanediamine dihydrochloride;
N¹-[2-(1-benzofuran-2-yl)-7-nitro-4-quinazolinyl]-N³,N³-dimethyl-1,3-propanediamine;
N¹-[2-(1-benzofuran-2-yl)-7-amino-4-quinazolinyl]-N³,N³-dimethyl-1,3-propanediamine dihydrochloride;
2-(1-benzofuran-2-yl)-4-{[3-(dimethylamino)propyl]amino}-7-quinazolinecarbonitrile;
2-(1-benzofuran-2-yl)-4-{[3-(dimethylamino)propyl]amino}-7-quinazolinecarboxamide dihydrochloride;
N¹-[2-(1-benzofuran-2-yl)pyrido[2,3-d]pyrimidin-4-yl]-N³,N³-dimethyl-1,3-propanediamine dihydrochloride;
N¹-[2-(1-benzofuran-2-yl)-8-methyl-4-quinazolinyl]-N³,N³-dimethyl-1,3-propanediamine dihydrochloride;
N¹-[2-(1-benzofuran-2-yl)-8-phenyl-4-quinazolinyl]-N³,N³-dimethyl-1,3-propanediamine dihydrochloride;
N¹-[2-(1-benzofuran-2-yl)-8-(trifluoromethyl)-4-quinazolinyl]-N³,N³-dimethyl-1,3-propanediamine dihydrochloride;
N¹-[2-(1-benzofuran-2-yl)-8-methoxy-4-quinazolinyl]-N³,N³-dimethyl-1,3-propanediamine dihydrochloride;
N¹-[2-(1-benzofuran-2-yl)-8-chloro-4-quinazolinyl]-N³,N³-dimethyl-1,3-propanediamine dihydrochloride;
N¹-[2-(1-benzofuran-2-yl)-8-nitro-4-quinazolinyl]-N³,N³-dimethyl-1,3-propanediamine;
N¹-[2-(1-benzofuran-2-yl)-8-amino-4-quinazolinyl]-N³,N³-dimethyl-1,3-propanediamine dihydrochloride;
2-(1-benzofuran-2-yl)-4-{[3-(dimethylamino)propyl]amino}-8-quinazolinecarbonitrile;
2-(1-benzofuran-2-yl)-4-{[3-(dimethylamino)propyl]amino}-8-quinazolinecarboxamide;
N¹-[2-(1-benzofuran-2-yl)benzo[g]quinazolin-4-yl]-N³,N³-dimethyl-1,3-propanediamine dihydrochloride;
N¹-[2-(1-benzofuran-2-yl)-6,7-dichloro-4-quinazolinyl]-N³,N³-dimethyl-1,3-propanediamine dihydrochloride;
N¹-[2-(1-benzofuran-2-yl)-6,8-dichloro-4-quinazolinyl]-N³,N³-dimethyl-1,3-propanediamine dihydrochloride;
N¹-[2-(1-benzofuran-2-yl)-6,8-dibromo-4-quinazolinyl]-N³,N³-dimethyl-1,3-propanediamine;
N¹-[2-(1-benzofuran-2-yl)-7,8-dimethyl-4-quinazolinyl]-N³,N³-dimethyl-1,3-propanediamine dihydrochloride;
N¹-[2-(1-benzofuran-2-yl)-7,8-dimethoxy-4-quinazolinyl]-N³,N³-dimethyl-1,3-propanediamine dihydrochloride; N¹,N¹-dimethyl-N³-[2-(3-methyl-1-benzofuran-2-yl)-4-quinazolinyl]-1,3-propanediamine dihydrochloride;
N¹-[2-(4-chloro-5-methoxy-1-benzofuran-2-yl)-4-quinazolinyl]-N¹,N¹-dimethyl-1,3-propanediamine hydrochloride;
N¹-[2-(5-methoxy-1-benzofuran-2-yl)-4-quinazolinyl]-N¹,N¹-dimethyl-1,3-propanediamine;
N¹,N¹-dimethyl-N³-[2-(5-methyl-1-benzofuran-2-yl)-4-quinazolinyl]-1,3-propanediamine dihydrochloride;
N¹,N¹-dimethyl-N³-[2-(5-chloro-1-benzofuran-2-yl)-4-quinazolinyl]-1,3-propanediamine;
N¹-[2-(5-bromo-1-benzofuran-2-yl)-4-quinazolinyl]-N³,N³-dimethyl-1,3-propanediamine;
N¹-[2-(6-methoxy-1-benzofuran-2-yl)-4-quinazolinyl]-N¹,N¹-dimethyl-1,3-propanediamine dihydrochloride;
N¹,N¹-dimethyl-N³-[2-(7-methyl-1-benzofuran-2-yl)-4-quinazolinyl]-1,3-propanediamine;
N¹,N¹-dimethyl-N³-[2-(7-methoxy-1-benzofuran-2-yl)-4-quinazolinyl]-1,3-propanediamine dihydrochloride;
N¹,N¹-dimethyl-N³-[8-methyl-2-(3-methyl-1-benzofuran-2-yl)-4-quinazolinyl]-1,3-propanediamine dihydrochloride;
N¹-[2-(5-methoxy-1H-indol-2-yl)-4-quinazolinyl]-N³,N³-dimethyl-1,3-propanediamine dihydrochloride;
N¹,N¹-dimethyl-N³-[2-(5-methoxy-1-methyl-1H-indol-2-yl)-4-quinazolinyl]-1,3-propanediamine dihydrochloride;
N¹-[2-(6-methoxy-1-benzofuran-2-yl)-4-quinazolinyl]-N¹,N¹-dimethyl-1,3-propanediamine dihydrochloride;
N¹-[2-(1H-indol-2-yl)-4-quinazolinyl]-N³,N³-dimethyl-1,3-propanediamine dihydrochloride;
N¹-[2-(1H-indol-2-yl)-4-quinazolinyl]-N-[3-(4-morpholinyl)propyl]amine dihydrochloride;
N¹,N¹-dimethyl-N³-[2-(1-methyl-1H-indol-2-yl)-4-quinazolinyl]-1,3-propanediamine dihydrochloride;
N¹-[2-(1-benzothien-2-yl)-4-quinazolinyl]-N³,N³-dimethyl-1,3-propanediamine dihydrochloride;
N¹,N¹-dimethyl-N³-[2-(3-quinolinyl)-4-quinazolinyl]-1,3-propanediamine dihydrochloride;
N¹,N¹-dimethyl-N³-[2-(2-naphthyl)-4-quinazolinyl]-1,3-propanediamine dihydrochloride;
2-(1-benzofuran-2-yl)-N³-[2-(1-methyl-2-pyrrolidinyl)ethyl]-4-quinazolinamine dihydrochloride;
2-(1-benzofuran-2-yl)-7,8-dimethyl-N-[2-(1-methyl-2-pyrrolidinyl)ethyl]-4-quinazolinamine dihydrochloride;
N¹-[2-(1-benzofuran-2-yl)-4-quinolinyl]-N³,N³-dimethyl-1,3-propanediamine dihydrochloride; and
N¹-[3-(1-benzofuran-2-yl)-1-isoquinolinyl]-N³,N³-dimethyl-1,3-propanediamine dihydrochloride.

10. A method of cancer prevention or therapy for treating cancers, which includes the step of administering to a subject in need of such therapy a therapeutically effective amount of compound of Formula I: 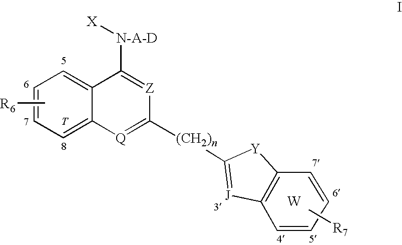
wherein;
D is selected from NR₁R₂ where R₁ and R₂ each independently represent H, lower C1-C6 alkyl or cycloalkyl optionally substituted with amino, hydroxyl or methoxy groups, or with one or more oxygen or nitrogen atoms as part of the cycloalkyl structure represents morpholine, pyrrolidine, piperidine, imidazole or 4-methylpiperazine;
n is selected from 0, 1 or 2;
X is selected from H or lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine;
Y is selected from O, CHR₃, S or, NR₄, where R₃ and R₄ each independently represent H or lower, C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine;
Z and Q represent N or CH, with the proviso that at least one of them is N;
J represents N or CR₅; where R₅ represents H or lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine,
A is (CH₂)ₙ where n is from 2 to 6, or A together with D forms a ring structure R₆ and R₇ at one or more of the available positions on rings T and W respectively, at each occurrence independently represent one or more H, halogen, C1-C4 alkyl, C1-C4 alkenyl, C1-C4 alkynyl, OR₈, SR₈, NR₈R₉, CH₂R₈, COR₈, SOR₈, SO₂R₈, SO₂NR₈R₉, CO₂R₈, CONR₈R₉, CF₃, CN, or NO₂, where R₈ and R₉ each independently represent H, lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine,
or a physiologically acceptable salt or phosphate prodrug or carboxylic acid or aminoacid ester prodrug thereof.

11. (canceled)

12. A method according to claim 10 wherein the subject is in need of restoration of its cell arrest function.

13-14. (canceled)

15. A method according to claim 10 wherein the method further includes the administration of one or more chemotherapeutic agents and/or therapies.

16-19. (canceled)

20. A pharmaceutical composition containing as an active agent a compound of formula I as defined in claim 1 and a pharmaceutically acceptable excipient, adjuvant, carrier, buffer or stabiliser.

21-26. (canceled)

27. A method of making a compound of formula I as defined in claim 10 wherein the method includes the steps of reacting a 2-aryl-4-chloroquinazoline of formula II with an amine: 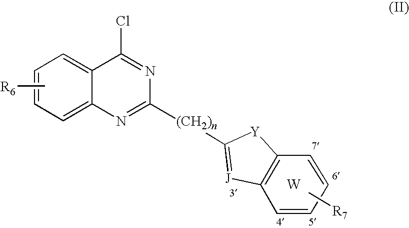
wherein:
n is selected from 0, 1 or 2;
Y is selected from O, CHR₃, S or, NR₄, where R₃ and R₄ each independently represent H or lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine;
J represents N or CR₅; where R₅ represents H or lower C1-C6 alkyl or cycloalkyl optionally substituted with amino, hydroxyl or methoxy groups, or with one or more oxygen or nitrogen atoms as part of the cycloalkyl structure may represent azetidine, pyrrolidine, piperidine, piperazine or morpholine,
R₆ and R₇ at one or more of the available positions on rings T and W respectively, at each occurrence independently represent one or more H, halogen, C1-C4 alkyl, C1-C4 alkenyl, C1-C4 alkynyl, OR₈, SR₈, NR₈R₉, CH₂R₈, COR₈, SOR₈, SO₂R₈, SO₂NR₈R₉, CO₂R₈, CONR₈R₉, CF₃, CN, or NO₂, where R₈ and R₉ each independently represent H, lower C1-C6 alkyl or cycloalkyl optionally substituted with amino, hydroxyl or methoxy groups, or with one or more oxygen or nitrogen atoms as part of the cycloalkyl structure represent azetidine, pyrrolidine, piperidine, piperazine or morpholine.

28. A method according to claim 27 wherein the method includes the steps of making a compound of formula II including the step of chlorination of a compound of formula III: 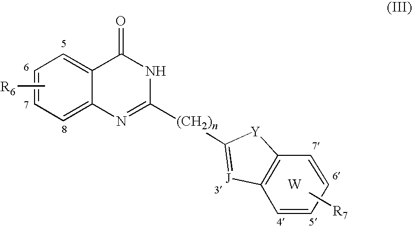
Wherein:
n is selected from 0, 1 or 2;
Y is selected from O, CHR₃, S or, NR₄, where R₃ and R₄ each independently represent H or lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine;
J represents N or CR₅; where R₅ represents H or lower C1-C6 alkyl or cycloalkyl optionally substituted with amino, hydroxyl or methoxy groups, or with one or more oxygen or nitrogen atoms as part of the cycloalkyl structure may represent azetidine, pyrrolidine, piperidine, piperazine or morpholine,
R₆ and R₇ at one or more of the available positions on rings T and W respectively, at each occurrence independently represent one or more H, halogen, C1-C4 alkyl, C1-C4 alkenyl, C1-C4 alkynyl, OR₈, SR₈, NR₈R₉, CH₂R₈, COR₈, SOR₈, SO₂R₈, SO₂NR₈R₉, CO₂R₈, CONR₈R₉, CF₃, CN, or NO₂, where R₈ and R₉ each independently represent H, lower C1-C6 alkyl or cycloalkyl optionally substituted with amino, hydroxyl or methoxy groups, or with one or more oxygen or nitrogen atoms as part of the cycloalkyl structure represents azetidine, pyrrolidine, piperidine, piperazine or morpholine.

29. A method according to claim 28 wherein the method includes the steps of making a compound of formula III including one of the following steps:
(i) by boronic acid (Suzuki) coupling;
(ii) by amination of a substituted anthranilate ester, followed by a cyclisation step;
(iii) by cyclisation of a substituted anthranilamide.

30-31. (canceled)

32. A compound of formula II as defined in claim 27.

33. A compound of formula III as defined in claim 28.

34. A compound according to claim 33 wherein formula III represents one of the following:
2-(6-methoxy-1-benzofuran-2-yl)-4(3H)-quinazolinone;
2-(2-naphthyl)-4(3H)-quinazolinone;
2-(3-quinolinyl)-4(3H)-quinazolinone;
2-(1-benzothien-2-yl)-4(3H)-quinazolinone;
2-(5-methoxy-1-benzofuran-2-yl)-4(3H)-quinazolinone;
2-(1-benzofuran-2-yl)-4(3H)-quinazolinone;
2-(1-benzofuran-2-yl)-5-chloro-4(3H)-quinazolinone;
2-(1-benzofuran-2-yl)-6-methyl-4(3H)-quinazolinone;
2-(1-benzofuran-2-yl)-6-(trifluoromethyl)-4(3H)-quinazolinone;
2-(1-benzofuran-2-yl)-6-fluoro-4(3H)-quinazolinone;
2-(1-benzofuran-2-yl)-6-chloro-4(3H)-quinazolinone;
2-(1-benzofuran-2-yl)-6-bromo-4(3H)-quinazolinone;
2-(1-benzofuran-2-yl)-6-nitro-4(3H)-quinazolinone;
2-(1-benzofuran-2-yl)-4-oxo-3,4-dihydro-6-quinazolinecarboxamide;
2-(1-benzofuran-2-yl)pyrido[3,4-d]pyrimidin-4(3H)-one;
2-(1-benzofuran-2-yl)-7-methyl-4(3H)-quinazolinone;
2-(1-benzofuran-2-yl)-7-fluoro-4(3H)-quinazolinone;
2-(1-benzofuran-2-yl)-7-chloro-4(3H)-quinazolinone;
2-(1-benzofuran-2-yl)-7-bromo-4(3H)-quinazolinone;
2-(1-benzofuran-2-yl)-7-nitro-4(3H)-quinazolinone;
2-(1-benzofuran-2-yl)-8-methyl-4(3H)-quinazolinone;
2-(1-benzofuran-2-yl)-8-methoxy-4(3H)-quinazolinone;
2-(1-benzofuran-2-yl)-8-chloro-4(3H)-quinazolinone;
2-(1-benzofuran-2-yl)-4-oxo-3,4-dihydro-8-quinazolinecarboxamide;
2-(1-benzofuran-2-yl)-6,7-dichloro-4(3H)-quinazolinone;
2-(1-benzofuran-2-yl)-7,8-dimethoxy-4(3H)-quinazolinone;
2-(3-methyl-1-benzofuran-2-yl)-4(3H)-quinazolinone;
8-methyl-2-(3-methyl-1-benzofuran-2-yl)-4(3H)-quinazolinone;
2-(5-methyl-1-benzofuran-2-yl)-4(3H)-quinazolinone;
2-(5-chloro-1-benzofuran-2-yl)-4(3H)-quinazolinone;
2-(5-bromo-1-benzofuran-2-yl)-4(3H)-quinazolinone;
2-(5-methoxy-1H-indol-2-yl)-4(3H)-quinazolinone;
2-(5-methoxy-1-methyl-1H-indol-2-yl)-4(3H)-quinazolinone;
2-(7-methyl-1-benzofuran-2-yl)-4(3H)-quinazolinone;
2-(7-methoxy-1-benzofuran-2-yl)-4(3H)-quinazolinone;
2-(1H-indol-2-yl)-4(3H)-quinazolinone;
2-(1-methyl-1H-indol-2-yl)-4(3H)-quinazolinone;
2-(1-benzofuran-2-yl)[3,2-d]pyrimidin-4(3H)-one;
2-(1-benzofuran-2-yl)-5-methyl-4(3H)-quinazolinone;
2-(1-benzofuran-2-yl)-5-nitro-4(3H)-quinazolinone;
2-(1-benzofuran-2-yl)-5-methoxy-4(3H)-quinazolinone;
2-(1-benzofuran-2-yl)pyrido[4,3-d]pyrimidin-4(3H)-one;
2-(1-benzofuran-2-yl)-6-methoxy-4(3H)-quinazolinone;
2-(1-benzofuran-2-yl)-7-(trifluoromethyl)-4(3H)-quinazolinone;
2-(1-benzofuran-2-yl)-7-methoxy-4(3H)-quinazolinone;
2-(1-benzofuran-2-yl)-4-oxo-3,4-dihydro-7-quinazolinecarboxamide;
2-(1-benzofuran-2-yl)pyrido[2,3-d]pyrimidin-4(3H)-one;
2-(1-benzofuran-2-yl)-8-phenyl-4(3H)-quinazolinone;
2-(1-benzofuran-2-yl)-8-(trifluoromethyl)-4(3H)-quinazolinone;
2-(1-benzofuran-2-yl)-8-nitro-4(3H)-quinazolinone;
2-(1-benzofuran-2-yl)benzo[g]quinazolin-4(3H)-one;
2-(1-benzofuran-2-yl)-6,8-dichloro-4(3H)-quinazolinone;
2-(1-benzofuran-2-yl)-6,8-dibromo-4(3H)-quinazolinone; and
2-(1-benzofuran-2-yl)-7,8-dimethyl-4(3H)-quinazolinone.

36. An assay for determining the restoration of cell arrest function including the steps of:
(a) plating and culturing one or more tumour cell lines in growth media under cell culture conditions,
(b) adding a compound of Formula I, as defined in claim 10, to one or more of the cultures,
(c) adding an inhibitor of cell division to one or more of the cultures,
(d) irradiating one or more of the cultures,
(e) incubating, harvesting, and
(f) analyzing the cellular DNA content profiles to estimate the proportions of G1-S— and G2/M-phase cells in the culture.
